(19) United States
(12) Patent Application Publication
(10) Pub. No.: US 2023/0338568 A1
(43) Pub. Date: Oct. 26, 2023

(54) NOVEL ISOMERIC COMPOUNDS COMPRISING A RING-OPENED THIOSUCCINIMIDE GROUP, AN OLIGOPEPTIDE FRAGMENT AND A CHIRAL MOIETY

(71) Applicant: GENEQUANTUM HEALTHCARE (SUZHOU) CO., LTD., Suzhou (CN)

(72) Inventors: Gang QIN, Suzhou (CN); Chong LIU, Suzhou (CN); Lehua HU, Suzhou (CN)

(21) Appl. No.: 18/001,764

(22) PCT Filed: Dec. 23, 2020
(86) PCT No.: PCT/CN2020/138509

§ 371 Date: Dec. 14, 2022

(51) Int. Cl. B01D 15/18; B01D 15/42; A61K 47/68; B01D 15/32

(52) U.S. Cl. CPC A61K 47/6855; A61K 47/6803; A61K 47/6889; B01D 15/1871; B01D 15/325; B01D 15/426

(57) ABSTRACT

It relates to the medicinal chemistry field, particularly to a process for separating the isomeric compounds comprising a ring-opened thiosuccinimide group and a chiral moiety.

### CROSS-REFERENCE TO RELATED APPLICATIONS

This application is a national stage entry filed under 35 U.S.C. § 371 of PCT international application No. PCT/CN2020/138509, filed on Dec. 23, 2020, the disclosure of which is hereby incorporated herein by reference in its entirety.

### TECHNICAL FIELD

The present disclosure relates to the medicinal chemistry field, particularly to isomeric compounds comprising a ring-opened thiosuccinimide group, an oligopeptide fragment and a chiral moiety with anti-cancer activity. The invention also relates to a process for separating the isomeric compounds.

### BACKGROUND

Increasing recurrence of mammalian tumors and severe side-effects of chemotherapeutic agents reduce the clinical efficacy of a large variety of anticancer agents that are currently being used. Thus, there is always a constant need to develop alternative or synergistic anticancer drugs with minimal side-effects. One important strategy to develop effective anticancer agents is to study into anticancer agents derived from “combination” of medicines, hundreds of trials are being conducted to evaluate combination of newer targeted drugs. Such targeted agents include antibody-drug-conjugates.

The concept of antibody-drug conjugates (ADCs) is not new, and as potent anti-tumor drugs based on monoclonal antibodies, they combine the advantages of antibodies (as a targeting moiety and/or a therapeutic moiety) and traditional cytotoxic drugs (with strong cytotoxicity).

An ideal linker of ADC must meet the following requirements: it should be sufficiently stable outside the cells to ensure the small-molecule drug is connected to the ligand; after entering the cell, the cleavable linker should break under appropriate conditions and release the active small molecule drugs; for the non-cleavable linker, the active component typically consists of a small molecule, a linker, and amino acid residues produced by the enzyme hydrolyzation of the ligands. Thus, in the development of ADCs, the linker design and the related coupling strategies are critical. These not only play a key role in the stabilization of the ADC, but also directly affect the biological activity, aggregation states, in vivo bioavailability, distribution and metabolism of the conjugate.

The current mainstream conjugation technology is chemical coupling strategy mainly based on the lysine or cysteine residues in the antibody. Due to the diversity in number and location of these amino acids in antibody which can react with linkers, the number and location of the cytotoxins in the ADCs are variable, and ADCs thus obtained are heterogenous. This heterogeneity will affect the quality, stability, effectiveness, metabolism and toxicity of ADCs. For example, in the drug instruction of Kadcyla, an ADC which was marketed in 2013, it clearly indicated that the number of cytotoxins in each antibody is between 0 and 8, the average n is about 3.5. The problem of heterogeneity of ADCs presents a major challenge in the development of a new generation of ADCs.

A defect of bioconjugates is off-target release which causes toxicity to normal tissues and reduces the number of effective bioconjugates at the target, resulting in reduced efficacy. More than half of the antibody-drug conjugates (ADCs) which are commercially available or in clinical trials use a thiosuccinimide structure (thiosuccinimide linkage) to couple the small molecule drug with the targeting antibody or protein. However, the thiosuccinimide linkage is not stable. In organisms, reverse Michael addition or exchange with other thiol groups may occur, directly leading to the fall-off of the cytotoxin from the ADC and resulting in off-target toxicity.

The ring-opening of the succinimide can increases the stability of the bioconjugates by eliminating the potential sites for reverse Michael addition or thiol exchange through reverse Michael addition mechanism.

The thiosuccinimide structure is formed by the reaction of a thiol group and a maleimide structure. In a situation with less or incomplete regioselectivity, the ring-opening reaction gives a pair of regioisomer. Furthermore, the covalent linkage of the thiol group to the succinimide results in a chiral center. The presence of diastereomers and enantiomers poses great challenge on the post processing of the ring-opening reaction. Therefore, further investigation for the isomers is required and there is an urgent need for new processes for purification.

### SUMMARY

Provided is a compound with the structure of any one of the following formulae (XI) to (XIV):

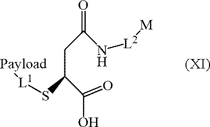

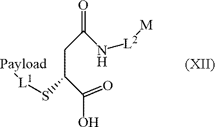

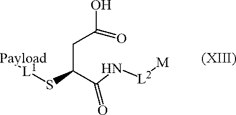

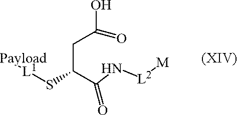

wherein Payload, L¹, L² and M are as defined below.

Also provided is an antibody drug conjugate (ADC) prepared using the compound of formula (XI), (XII), (XIII) or (XIV) and an antibody or the antigen binding fragment thereof.

In a particular embodiment, the compound of formulae (XI) to (XIV) has the structure of formulae (i) to (iv), respectively.

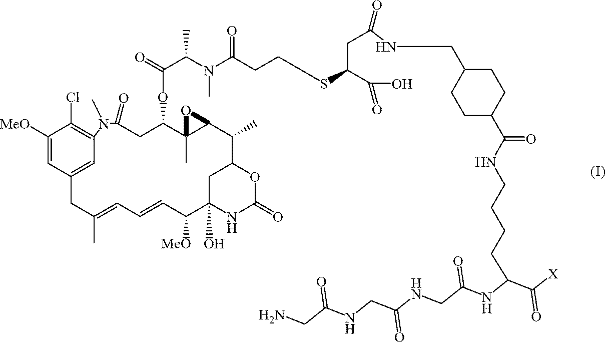

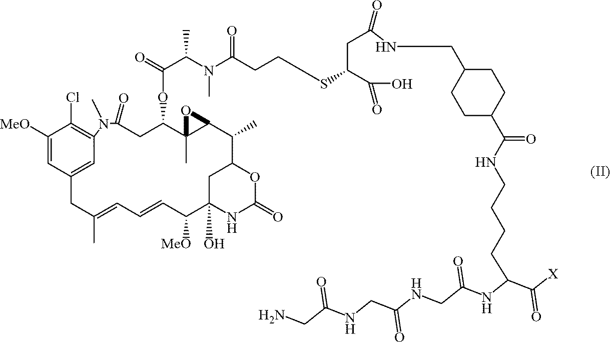

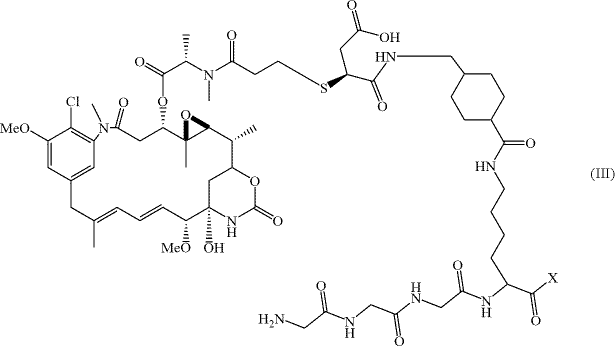

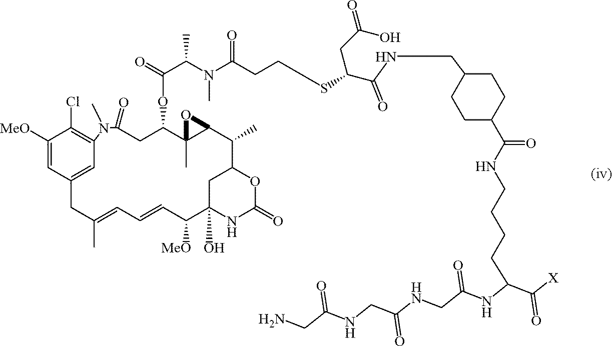

wherein x is —OH or —NH₂.

The mixture of ADCs obtained by conjugating compound of formula (iii) or compound of formula (iv) with therapeutic antibody is denoted as “α-ADCs”.

α-ADCs comprising a ring-opened thiosuccinimide group, an oligopeptide fragment and a chiral moiety have been made, and their bioactivities have demonstrated by in vitro and in vivo assays. Unexpected anti-cancer results are as following:
α-ADCs have significant inhibitory effect on the proliferation of HER2-positive cells, and its effect is tens of times better than DM1.
α-ADCs can be enriched to tumor within 24 hrs and maintain in situ stability.

In conclusion, under the current study conditions, repeated intravenous infusion (once every 3 weeks, total 3 doses) with α-ADCs at 10, 30 and 45 mg/kg could be tolerated in cynomolgus monkeys at a dose up to 45 mg/kg. The highest non-severe toxicity dose (HNSTD) was determined as 45 mg/kg, comparing Kadcyla showed toxicity dose at 10 mg/kg.

Optimized molecular structure improved metabolism. The lower dose of the α-ADCs caused similar efficacy compared to Kadcyla.

Also provided is a process for separating one or more target compounds from Mixture 1 comprising four compounds, each of the four compounds comprising Moiety 1, Moiety 2 and Moiety 3;

wherein Moiety 1 has a ring-opened thiosuccinimide structure selected from formulae (I) to (IV):

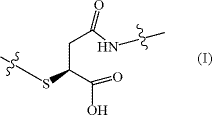

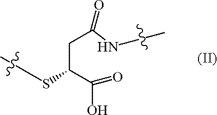

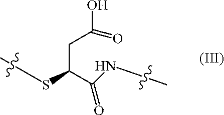

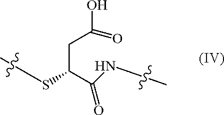

wherein
the thiol group and the amido group in Moiety 1 constitutes two linking sites, through one of which Moiety 2 is linked to Moiety 1, and through the other of which Moiety 3 is linked to Moiety 1;
   Moiety 2 contains one or more chiral centers;
   Moiety 1 in the four compounds is different;
   Moiety 3 is the rest moiety of the molecule;
   the weight of Moiety 3 is no more than 1900;
the one or more target compounds are selected from the said four compounds comprised by Mixture 1;
   the process comprising the following steps (1) and (2)
   (1) providing Mixture 1;
   (2) subjecting Mixture 1 to chromatography so as to obtain the target compound(s), wherein
      a. each of the target compound(s) are obtained in a separate product, or
      b. two target compounds are obtained in a mixture, which is defined as Mixture 2.

In an embodiment, the process further comprises the steps (3) and (4)
(3) recovering an eluate collected in step (2) comprising Mixture 3, wherein Mixture comprises one or more additional target compounds which are different from those separated in step (2);
(4) subjecting the eluate recovered in step (3) to chromatography to separate the additional target compound(s).

Formulae (I) and (II) are referred to as β, and the configuration of formulae (III) and (IV) are referred to as α, according to the position of the thiol group.

Also provided is a process for analyzing one or more compounds in Mixture 1 which comprises four compounds, each of the four compounds in the mixture comprising Moiety 1, Moiety 2 and Moiety 3.

Also provided is a process for analyzing one or more compounds in Mixture 2 which comprises two compounds, each of the two compounds in the mixture comprising Moiety 1, Moiety 2 and Moiety 3.

### BRIEF DESCRIPTION OF THE DRAWINGS

FIG. 1: HPLC Graph for separation of β1, β2, and α isomers; using Daisopak C4 SP-120-8-C4-P.

FIG. 2: HPLC Graph for separation of β1, β2, and α isomers; using Kromasil C18 100-10C18.

FIG. 3: HPLC Graph for separation of β1, β2, and α isomers; using Acchrom C18 C18-ME 10 um 100 Å.

FIG. 4: HPLC Graph for separation of β1, β2, and α isomers; using Daisopak C18 100-10-ODS-P.

FIG. 5: HPLC Graph for separation of β1, β2, and α isomers; Eluent A: 0.1%TFA in water, Eluent B: MeOH.

FIG. 6: HPLC Graph for separation of β1, β2, and α isomers; Eluent A: 0.1% H₃PO₄ in water, Eluent B: MeOH.

FIG. 7: HPLC Graph for separation of β1, β2, and α isomers; Eluent A: 0.2% TFA in water, Eluent B: MeOH.

FIG. 8: HPLC Graph for separation of β1, β2, and α isomers; Eluent A: 0.1% CH₂O₂ in water, Eluent B: MeOH.

FIG. 9: HPLC Graph for separation of β1, β2, and α isomers; Eluent A: 0.2% CH₂O₂, Eluent B: MeOH.

FIG. 10: HPLC Graph for separation of β1, β2, and α isomers; Eluent A: 0.5% AA in water, Eluent B: MeOH.

FIG. 11: HPLC Graph for separation of β1, β2, and α isomers; Eluent A: 0.1% CH₃COOH in water, Eluent B: MeOH.

FIG. 12: HPLC Graph for separation of β1, β2, and α isomers; Eluent A: 0.3% CH₃COOH in water, Eluent B: MeOH.

FIG. 13: HPLC Graph for separation of β1, β2, and α isomers; Eluent A: 0.3% CH₃COOH + 0.05% L-TA in water, Eluent B: MeOH.

FIG. 14: HPLC Graph for separation of β1, β2, and α isomers; Eluent A: 10 mM NaH₂PO₄ PH=2.0, Eluent B: MeOH.

FIG. 15: HPLC Graph for separation of β1, β2, and α isomers; Eluent A: 10 mM NaH₂PO₄ PH=4.0, Eluent B: MeOH.

FIG. 16: HPLC Graph for separation of β1, β2, and α isomers; Eluent A: 10 mM NaH₂PO₄ PH=6.0, Eluent B: MeOH.

FIG. 17: HPLC Graph for separation of β1, β2, and α isomers; Eluent A: 10 mM K₂HPO₄ PH=4.0, Eluent B: MeOH.

FIG. 18: HPLC Graph for separation of β1, β2, and α isomers; Eluent A: 10 mM TEAP PH=4.0, Eluent B: MeOH.

FIG. 19: HPLC Graph for separation of β1, β2, and α isomers; Eluent A: 10 mM NaClO₄ PH=2.0, Eluent B: MeOH.

FIG. 20: HPLC Graph for separation of β1, β2, and α isomers; Eluent A: 0.3% CH₃COOH in water, Eluent B: MeOH; gradient: 46%-66% B over 30 min.

FIG. 21: HPLC Graph for separation of β1, β2, and α isomers; Eluent A: 0.3% CH₃COOH in water, Eluent B: MeOH; gradient: 46%-56% B over 80 min.

FIG. 22: HPLC Graph for analysis of β1, β2, and α isomers; using Phenomenex Gemini 110 Å 5 um 250*4.6 mm.

FIG. 23: HPLC Graph for separation of α1 and α2 isomers; using Kromasil 100-10C18, 21.2*250 mm (100 Å, 10 µm); Eluent C: 0.1% TFA in water, Eluent D: 0.1% TFA in ACN.

FIG. 24: HPLC Graph for separation of α1 and α2 isomers; Eluent C: 0.1%TFA in water, Eluent D: ACN.

FIG. 25: HPLC Graph for separation of α1 and α2 isomers; Eluent C: 0.1% AcOH in water, Eluent D: ACN.

FIG. 26: HPLC Graph for separation of α1 and α2 isomers; Eluent C: 0.3% AcOH in water, Eluent D: ACN.

FIG. 27: HPLC Graph for separation of α1 and α2 isomers; Eluent C: 0.1% CH₂O₂ in water, Eluent D: ACN.

FIG. 28: HPLC Graph for separation of α1 and α2 isomers; Eluent C: 0.3% CH₂O₂ in water, Eluent D: ACN.

FIG. 29: HPLC Graph for separation of α1 and α2 isomers; Eluent C: 0.5% AA in water, Eluent D: ACN.

FIG. 30: HPLC Graph for separation of α1 and α2 isomers; Eluent C: 0.1% H₃PO₄ in water, Eluent D: ACN.

FIG. 31: HPLC Graph for separation of α1 and α2 isomers; Eluent C: 0.2% H₃PO₄ in water, Eluent D: ACN.

FIG. 32: HPLC Graph for separation of α1 and α2 isomers; Eluent C: 10 mM NaH₂PO₄ PH=2.0, Eluent D: ACN.

FIG. 33: HPLC Graph for separation of α1 and α2 isomers; Eluent C: 10 mM NaH₂PO₄ PH=4.0, Eluent D: ACN.

FIG. 34: HPLC Graph for separation of α1 and α2 isomers; Eluent C: 10 mM NaH₂PO₄ PH=6.0, Eluent D: ACN.

FIG. 35: HPLC Graph for separation of α1 and α2 isomers; Eluent C: 10 mM K₂HPO₄ PH=2.0, Eluent D: ACN.

FIG. 36: HPLC Graph for separation of α1 and α2 isomers; Eluent C: 10 mM K₂HPO₄ PH=4.0, Eluent D: ACN.

FIG. 37: HPLC Graph for separation of α1 and α2 isomers; Eluent C: 10 mM K₂HPO₄ PH=6.0, Eluent D: ACN.

FIG. 38: HPLC Graph for separation of α1 and α2 isomers; Eluent C: 10 mM K₂HPO₄ PH=8.0, Eluent D: ACN.

FIG. 39: HPLC Graph for separation of α1 and α2 isomers; Eluent C: 30 mM NaH₂PO₄ PH=2.0, Eluent D: ACN.

FIG. 40: HPLC Graph for separation of α1 and α2 isomers; Eluent C: 30 mM NaH₂PO₄ PH=6.0, Eluent D: ACN.

FIG. 41: HPLC Graph for separation of α1 and α2 isomers; Eluent C: 30 mM NaH₂PO₄ PH=4.0, Eluent D: ACN.

FIG. 42: HPLC Graph for separation of α1 and α2 isomers; Eluent C: 10 mM TEAP PH=6.0, Eluent D: ACN.

FIG. 43: HPLC Graph for separation of α1 and α2 isomers; Eluent C: 10 mM TEAP PH=2.0, Eluent D: ACN.

FIG. 44: HPLC Graph for separation of α1 and α2 isomers; Eluent C: 10 mM TEAP PH=4.0, Eluent D: ACN.

FIG. 45: HPLC Graph for analysis of α1 and α2 isomers; using Waters X-select, CSH Phenyl-Hexyl 3.5um 150*4.6 mm.

FIG. 46: The effects of isomers on proliferation of cancer cells.

FIG. 47: The effects of α-ADCs on proliferation of cancer cells.

FIG. 48: Bio-distribution characteristics of α-ADCs (PET/CT scan result after single intravenous infusion of ⁸⁹Zr-α-ADCs).

FIG. 49: In vivo anti-tumor efficacy of α-ADCs. The x-axis represents time after single dose. The y-axis represents tumor sizes.

FIG. 50: Pharmacokinetic characteristics of α-ADCs. The x-axis represents time after single intravenous infusion. The y-axis represents serum concentration.

FIG. 51: Summarized toxicokinetic data of α-ADCs. The x-axis represents time after the first dose. The 0^{th}, 504^{th} and 1008^{th} hour represents the time points for repeated doses of intravenous infusion. The 1008^{th} to the 2016^{th} hour represents the 6-week recovery period. The y-axis represents serum concentration.

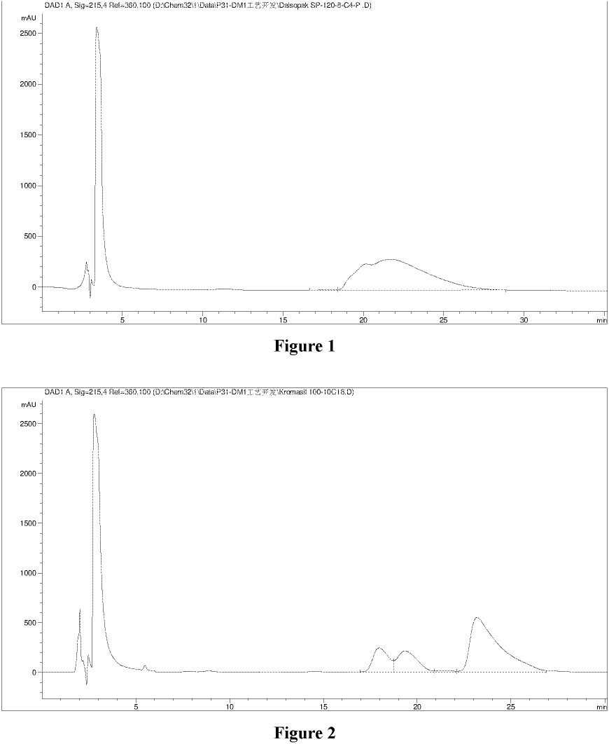

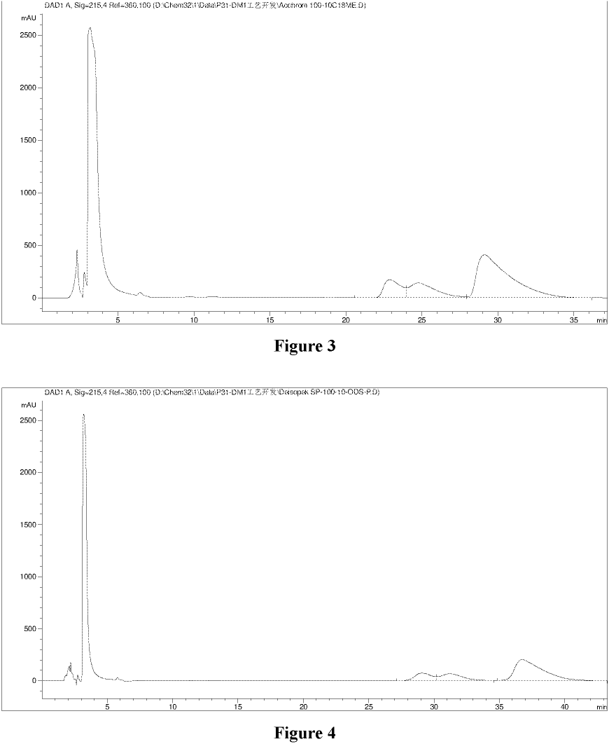

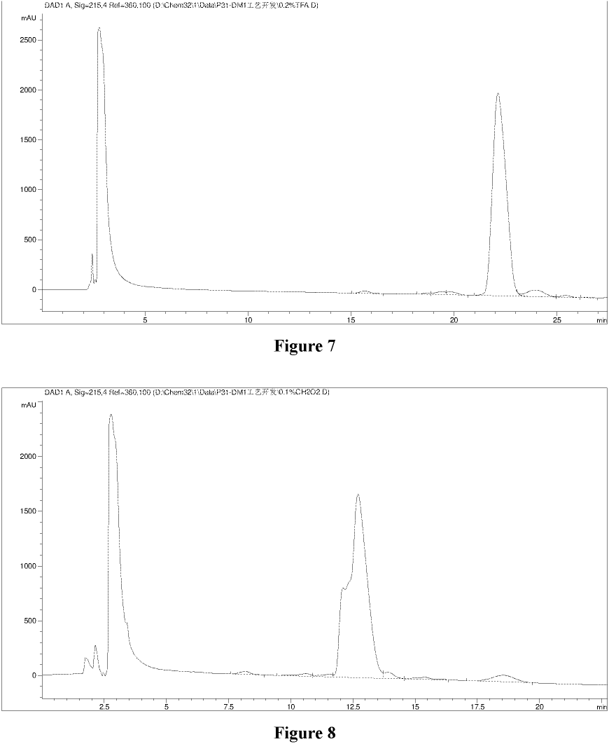

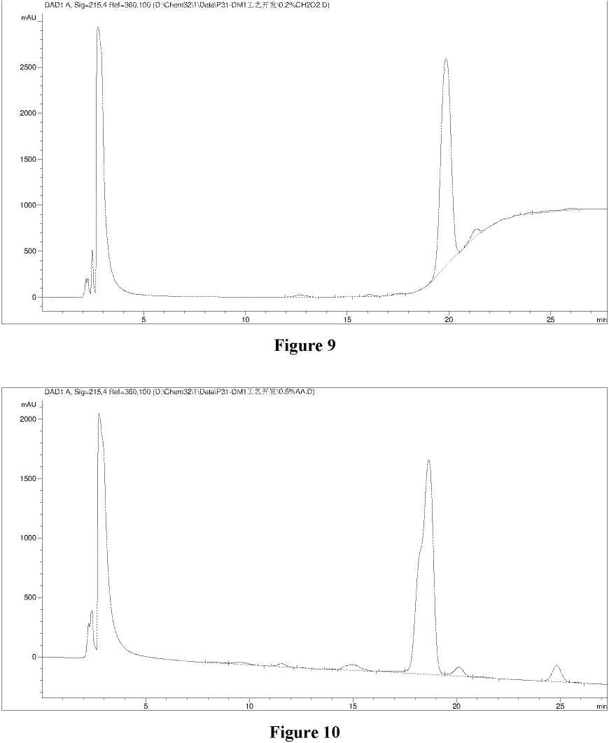

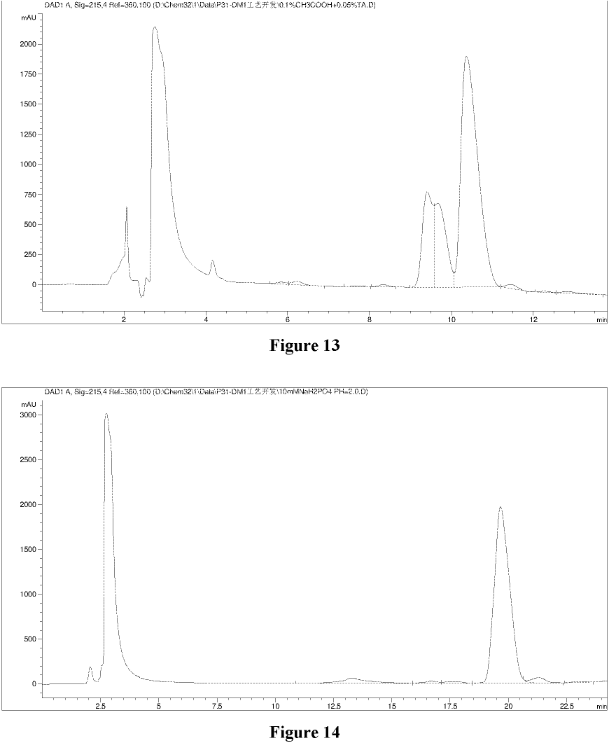

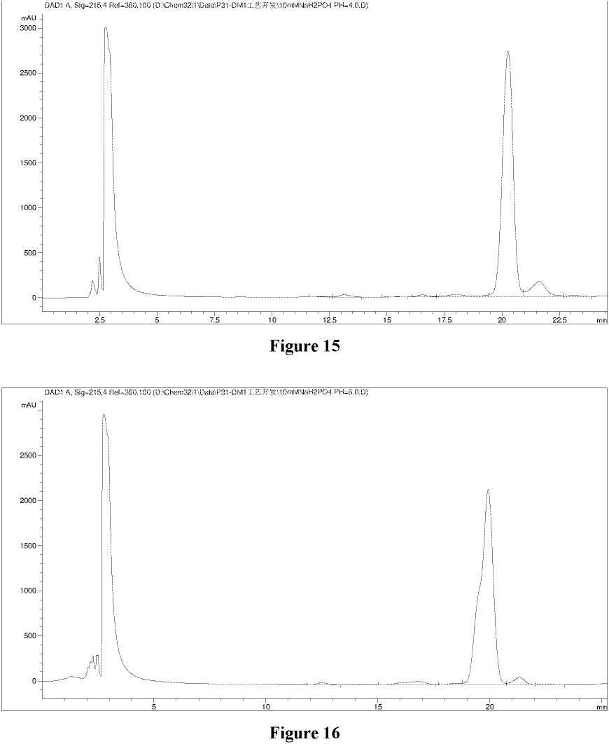

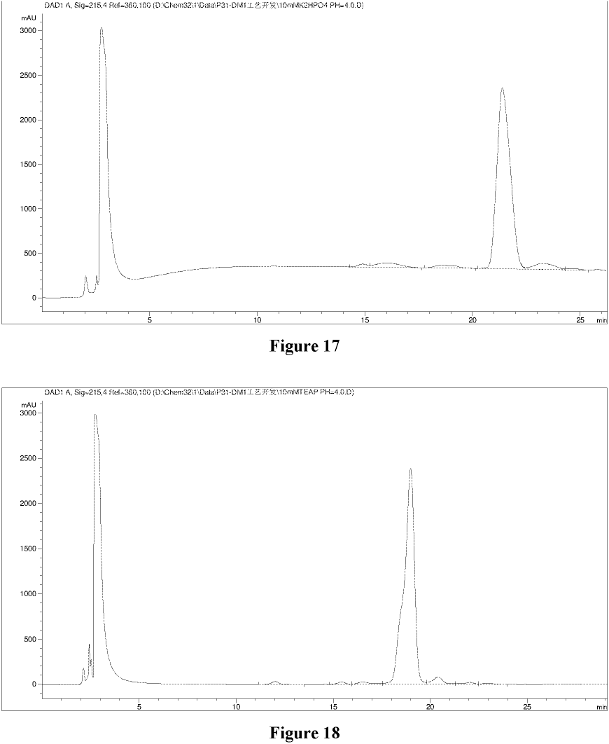

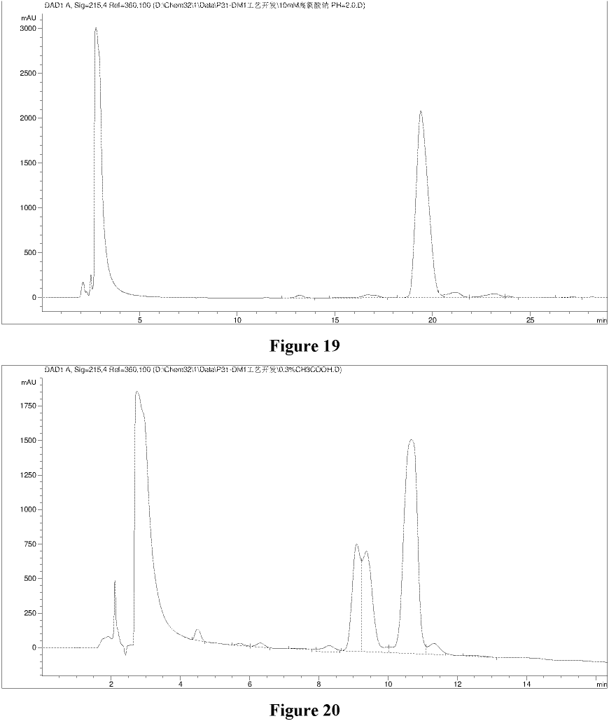

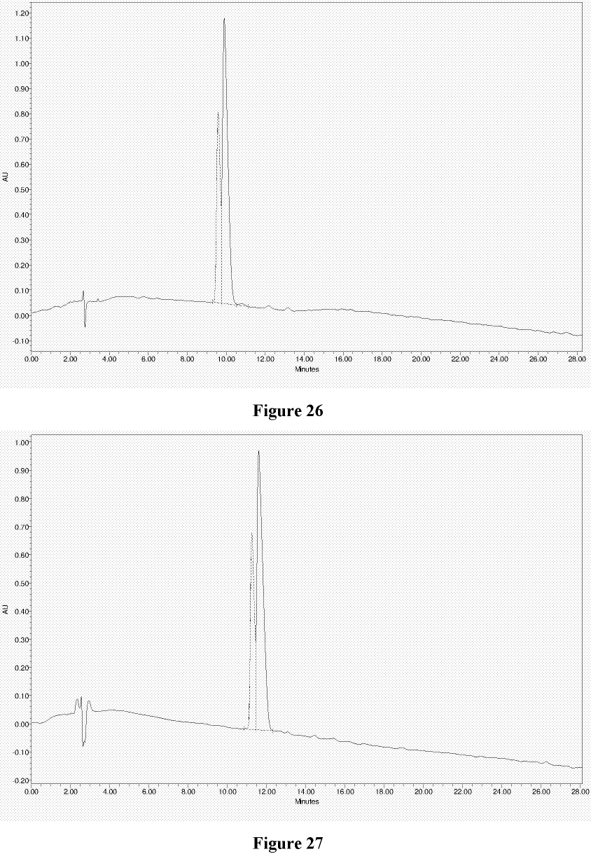

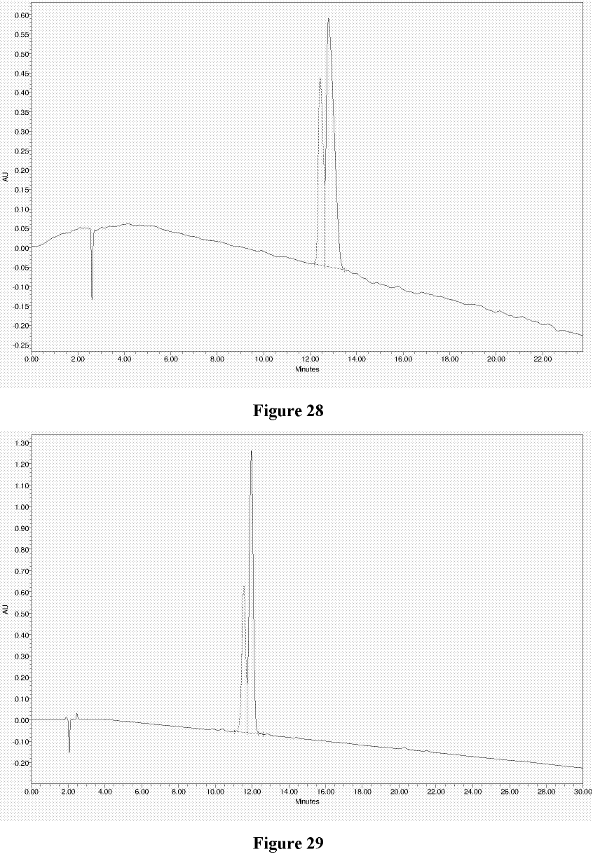

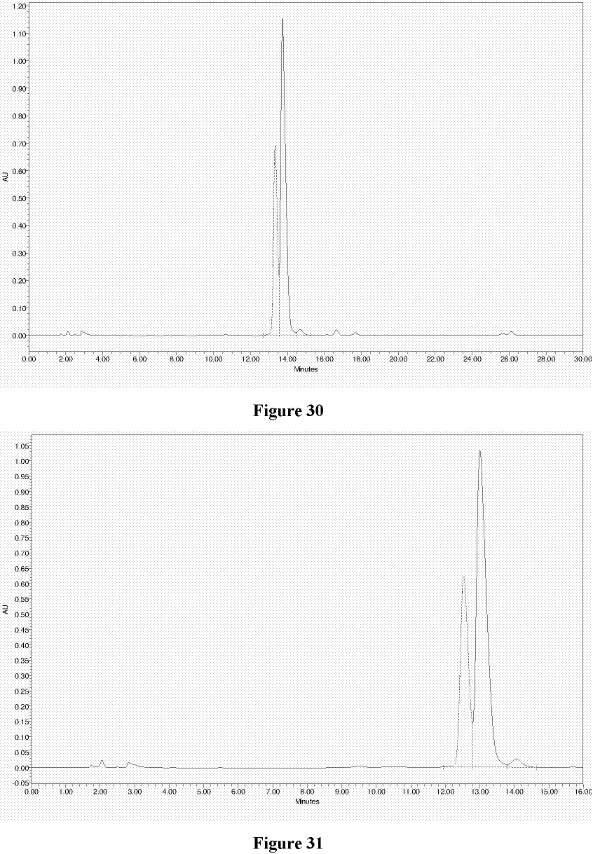

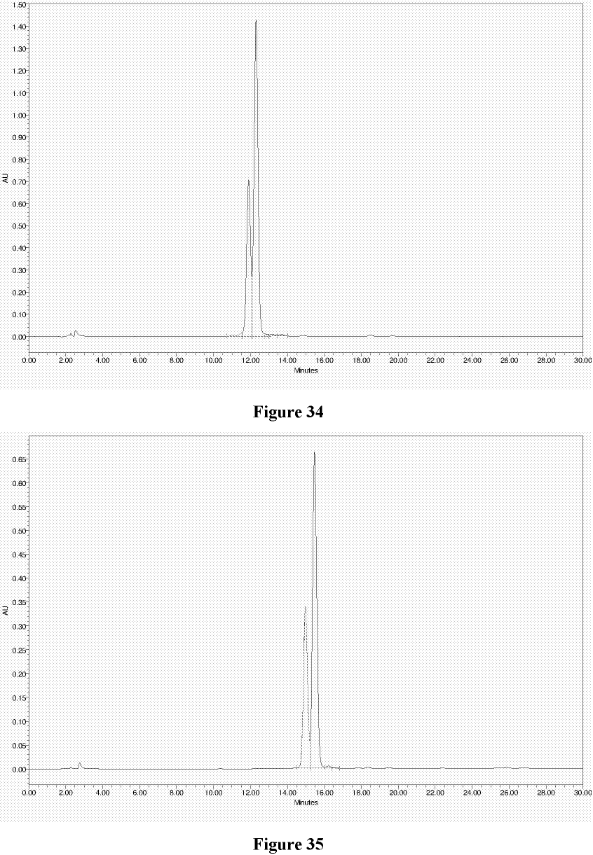

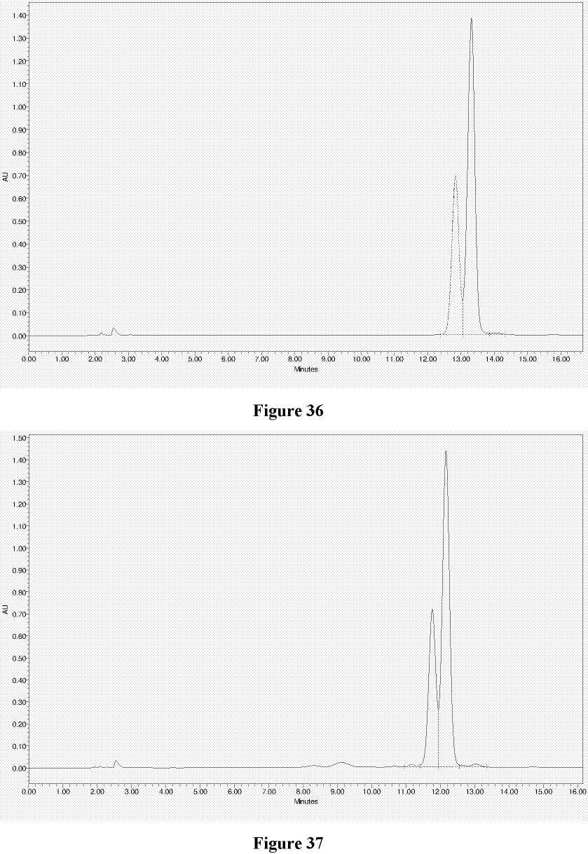

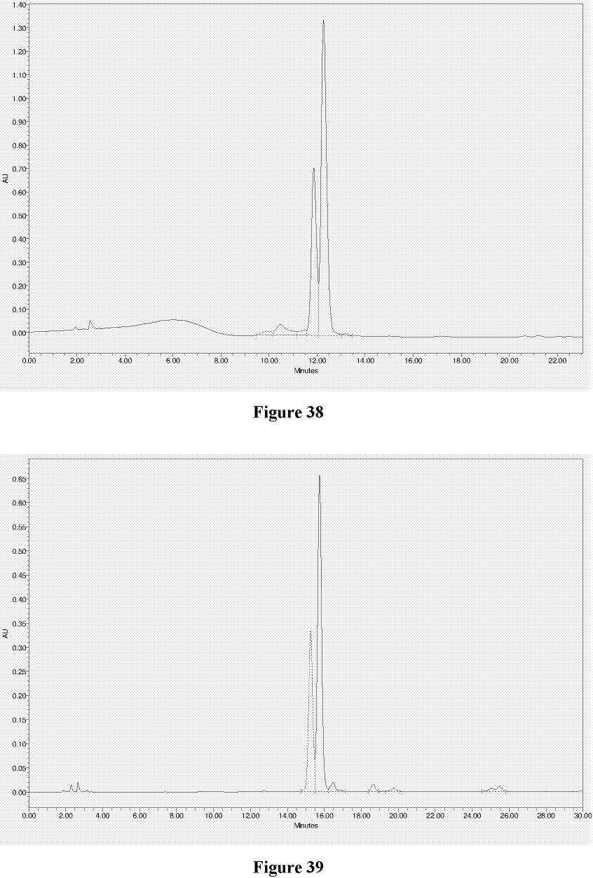

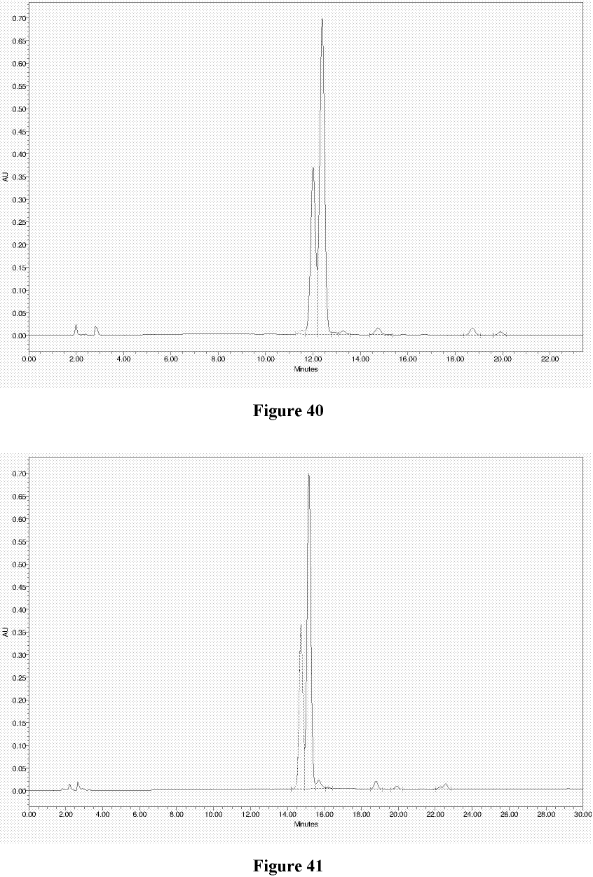

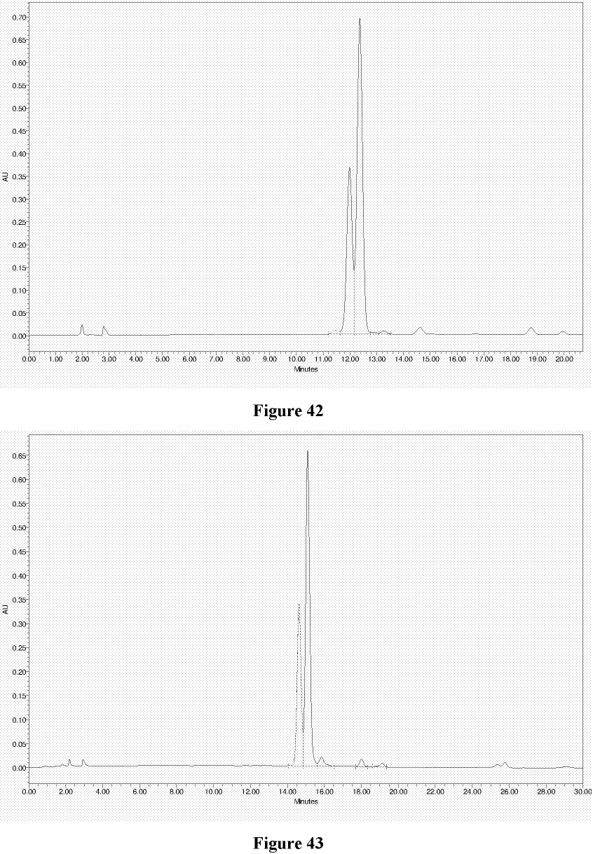

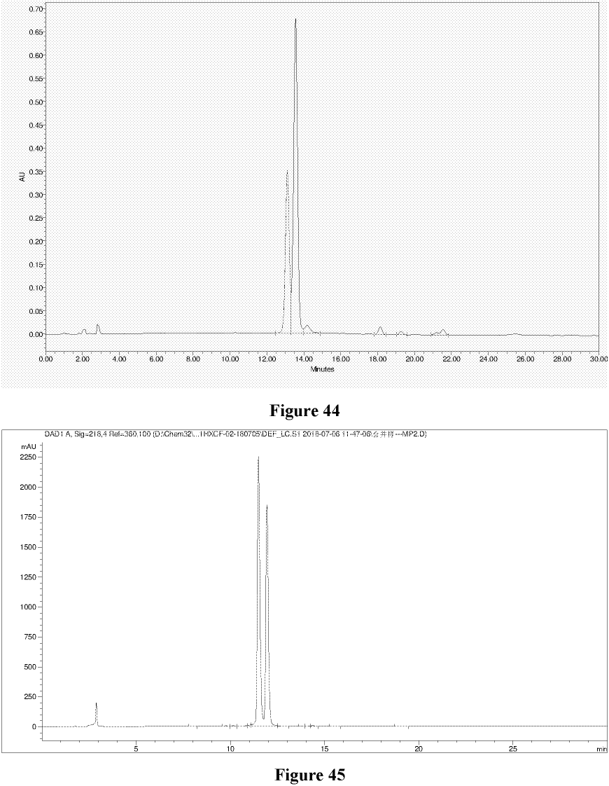

In FIGS. 1-45, x-axis represents time (minutes). In FIGS. 1-21 and 45, y-axis represents UV absorbance (mAU), and in FIGS. 22-44, y-axis represents UV absorbance (AU).

### DETAILED DESCRIPTION

### General Definitions

Unless otherwise defined hereinafter, all technical and scientific terms used herein have the same meaning as commonly understood by those skilled in the art. The techniques used herein refer to those that are generally understood in the art, including the variants and equivalent substitutions that are obvious to those skilled in the art. While the following terms are believed to be readily comprehensible by those skilled in the art, the following definitions are set forth to better illustrate the present disclosure. When a trade name is present herein, it refers to the corresponding commodity or the active ingredient thereof. All patents, published patents applications and publications cited herein are hereby incorporated by reference.

When a certain amount, concentration, or other value or parameter is set forth in the form of a range, a preferred range, or a preferred upper limit or a preferred lower limit, it should be understood that it is equivalent to specifically revealing any range formed by combining any upper limit or preferred value with any lower limit or preferred value, regardless of whether the said range is explicitly recited. Unless otherwise stated, the numerical ranges listed herein are intended to include the endpoints of the range and all integers and fractions (decimals) within the range. For example, the expression “about 0.01% to about 1%” means any values between 0.01% and 1%, for example 0.01%, 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95% and 1%. Other similar expressions like “40%-50% to about 50%-70%” should also be understood in a similar manner.

Unless otherwise stated herein, singular forms like “a” and “the” include the plural forms. The expression “one or more” or “at least one” may mean 1, 2, 3, 4, 5, 6, 7, 8, 9 or more.

The terms “about” and “approximately”, when used in connection with a numerical variable, generally mean that the value of the variable and all values of the variable are within experimental error (for example, within a 95% confidence interval for the mean) or within ± 10% of a specified value, or a wider range.

The term “mixture” is intended to mean a mixture containing more than one species of compounds, wherein one or more species of compounds can be target compound(s). The term “target compound” means a compound to be separated or purified. When defining a separation process, the species of the target compound(s) are determined before the separation operations. It is to be understood that the product which contains the target compound(s) could be in any desired form, for example a product containing a pure isomer compound or a mixture containing a plurality of predefined species of the target compounds.

The term “stoichiometric ratio” means matching various substances according to a certain amount by weight.

The term “optional” or “optionally” means the event described subsequent thereto may, but not necessarily happen, and the description includes the cases wherein the said event or circumstance happens or does not happen.

The expression “comprising” or similar expressions “including,” “containing” and “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, plus elements, steps or ingredients that are optionally present that do not substantially affect the essential and novel characteristics of the claimed subject matter. It should be understood that the expression “comprising” encompasses the expressions “consisting essentially of” and “consisting of”.

The chemical bond in the compound of the disclosure can be depicted herein with a solid line

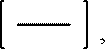

a wavy line

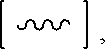

a solid wedge

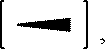

or a dashed wedge

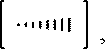

It is intended that a bond to an asymmetric atom depicted with a solid line indicates that all possible stereoisomers at the atom (e.g., specific enantiomers, racemic mixtures and the like) are contemplated. It is intended that a bond to an asymmetric atom depicted with a wavy line indicates that the bond is either a solid wedge

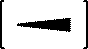

bond or a dashed wedge

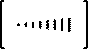

bond. It is intended that a bond to an asymmetric atom depicted with a solid or dashed wedge indicates the existence of the stereoisomer that is shown. When present in a racemic mixture, a solid or dashed wedge is used to define relative stereochemistry rather than absolute stereochemistry. Unless otherwise indicated, it is intended that the compound of the disclosure can be present in the form of stereoisomers (including cis- and trans- isomers, optical isomers (e.g., R and S enantiomers), diastereomers, geometric isomers, rotamers, conformers, atropisomers, and mixtures thereof). The compound of the disclosure can exhibit one or more types of the above isomerism, and can be consisted of a mixture thereof (e.g., a racemic mixture and/or a diastereomeric pair).

The term “hydrocarbyl” refers to a monovalent radical derived from a hydrocarbon. Examples of hydrocarbyls include but not limited to alkyl, alkenyl, alkynyl, cycloalkyl, and aryl.

The term “alkyl” refers to a straight or branched saturated aliphatic hydrocarbon group consisting of carbon atoms and hydrogen atoms, which is connected to the rest of the molecule through a single bond. The alkyl group may contain 1 to 20 carbon atoms, referring to C₁-C₂₀ alkyl group, for example, C₁-C₄ alkyl group, C₁-C₃ alkyl group, C₁-C₂ alkyl, C₃ alkyl, C₄ alkyl, C₃-C₆ alkyl. Non-limiting examples of alkyl groups include but are not limited to methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethyl butyl, 1,1-dimethylbutyl, 2,3-dimethylbutyl, 1,3-dimethylbutyl or 1,2-dimethylbutyl, or their isomers.

The term “radical valence” refers to the property of the radical being monovalent, divalent or trivalent. A bivalent radical refers to a group obtained from the corresponding monovalent radical by removing one hydrogen atom from a carbon atom with free valence electron(s), and a trivalent radical refers to a group obtained from the corresponding bivalent radical by removing one hydrogen atom from a carbon atom with free valence electron(s). For example, an “alkylene” or an “alkylidene” refers to a saturated divalent hydrocarbon group, either straight or branched. Examples of alkylene groups include but are not limited to methylene (—CH₂—), ethylene (—C₂H₄—), propylene (—C₃H₆—), butylene (—C₄H₈—), pentylene (—C₅H₁₀—), hexylene (—C₆H₁₂—), 1-methylethylene (—CH(CH₃)CH₂—), 2-methylethylene (—CH₂CH(CH₃)—), methylpropylene, ethylpropylene. Examples of cycloalkylene groups include but are not limited to divalent monocyclic alkyl groups such as cyclopropylene, cyclobutylene, cyclopentylene, cyclohexylene, cycloheptylene and cyclooctylene, and divalent multicyclic alkyl groups containing fused, spiro or bridged rings. Examples of arylene groups include but are not limited to phenylene. Examples of heterocyclylene groups include but are not limited to pyrrolidinylene, imidazolidinylene, pyrazolidinylene, piperidinylene, piperazinylene and morpholinyl. Examples of heteroarylene groups include but are not limited to pyrrolylene, furanylene, thienylene, imidazolylene, oxazolylene, oxadiazolylene, oxatriazoleene, isoxazolylene, thiazolylene, thiadiazolylene, isothiazolylene, pyrazolylene, triazolylene and tetrazolylene.

The term “ring-opened thiosuccinimide group” represents a product resulting from the opening of the succinimide ring in the thiosuccinimide group. The ring-opening reaction of the thiosuccinimide group can occur through breakage of any one of the two amido bonds in the thiosuccinimide group, resulting in two isomers. In particular, the ring-opened thiosuccinimide group is selected from

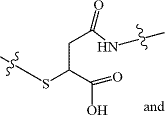

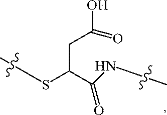

preferably

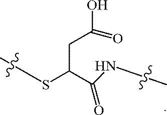

The term “regioselectivity” refers to the favoring of a reagent to one atom over another and the reaction to produce one regioisomer over another, in a chemical reaction that could happen on multiple sites of a specific molecule. “Regiospecific” refers to the selectivity of a chemical reaction or a reagent to only one of two or more possible sites on a specific molecule.

The term “diastereomer” refers to a stereoisomer in which the molecule has two or more chiral centers and there is a non-mirror relationship between the molecules.

A small molecule compound refers to a molecule with a size comparable to that of an organic molecule commonly used in medicine. The term does not encompass biological macromolecules (e.g., proteins, nucleic acids, etc.), but encompasses low molecular weight peptides or derivatives thereof, such as dipeptides, tripeptides, tetrapeptides, pentapeptides, and the like. Typically, the molecular weight of the small molecule compound can be, for example, about 100 to about 2000 Da, about 200 to about 1000 Da, about 200 to about 900 Da, about 200 to about 800 Da, about 200 to about 700 Da, about 200 to about 600 Da, about 200 to about 500 Da. As used herein, a small molecule compound may also be known as a drug.

As used herein, the term “antibody (Ab)” is an immunoglobulin (Ig) molecule or a derivative thereof that specifically binds to an antigen through at least one antigen-binding site. A “conventional” or “full-length” antibody typically consists of four polypeptides: two heavy chains (HC) and two light chains (LC). As used herein, the definition of “antibody” includes but not limit to conventional antibodies, non-human antibodies, humanized antibodies, chimeric antibodies, diabodies or nanobodies (i.e., single-domain antibodies, VHH domains). Also contemplated are members of any immunoglobulin type (e.g., IgG, IgM, IgD, IgE, IgA and IgY), any class (e.g., IgG1, IgG2, IgG3, IgG4, IgAl and IgA2) or subclass (e.g., IgG2a and IgG2b), or any derivatives thereof.

As used herein, an “antibody fragment” of an antibody refers to any portion of an antibody comprising fewer amino acid residues than a full-length antibody, such as an antigen-binding fragment that contains at least a portion of the variable domains (e.g., one or more CDRs) of the antibody and specifically binds to the same cognate antigen as the full-length antibody, or an Fc fragment that contains heavy chain constant regions of the antibody and binds to Fc receptors on the cell surface. Antibody fragments can be obtained by various methods, such as chemical or enzymatic treatment, chemical synthesis or recombinant DNA technology. Examples of antibody fragments include, but are not limited to, Fv (the fragment variable region), scFv (single-chain Fv fragment), dsFv (disulfide-stabilized variable fragment), scdsFv (single-chain disulfide-stabilized variable fragment), diabody, Fd (the fragment difficult), Fab (the fragment antigen binding), scFab (single-chain Fab), Fab′, F(ab′)2, Fc (the fragment crystallizable region) and any derivatives thereof.

HER2 refers to human epidermal growth factor receptor-2, which belongs to the epidermal growth factor (EGFR) receptor tyrosine kinase family. In the present application, the terms ErbB2 and HER2 have the same meaning and can be used interchangeably.

As used herein, the term “natural amino acid” refers to an amino acid that is a protein constituent amino acid, including the common twenty amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine), and the less common selenocysteine and pyrrolysine.

As used here, the term “unnatural amino acid” or “non-natural amino acid” refers to an amino acid that is not a protein constituent amino acid. In particular, the term refers to an amino acid that is not a natural amino acid as defined above.

In specifying amino acid sequences herein, standard one-letter amino acid codes are used, unless otherwise specified. Thus, for example, LPXTG represents the sequence -Leu-Pro-X-Thr-Gly-, wherein X is any natural or non-natural single amino acid residue, LPETG represents -Leu-Pro-Glu-Thr-Gly-, LPETGG represents -Leu-Pro-Glu-Thr-Gly-Gly-, and GGG represents -Gly-Gly-Gly-.

As used herein, the term “peptidomimetic” refers to a compound that mimics the conformation and desirable features of a particular peptide.

As used herein, a “receptor” refers to a structure inside or on the surface of a cell that binds a specific substance and causes a specific effect in the cell. Receptors may include T-cell receptors, B-cell receptors, and receptors of signaling molecules, cell growth factors and cytokines as described herein.

As described herein, the interaction between chemical entities (e.g., ions, molecules, groups or radicals) means the non-covalent interactions, including but not limit to hydrogen bonding, π-π stacking, ionic interaction, ion-induced dipole forces, and ion-dipole forces.

As used herein, the term “ionize” refers to dissociating one or more acidic hydrogens in an acidic group so as to form a radical bearing one or more negative charges, or combining one or more protons to an basic group so as to form a radical bearing one or more positive charges. For example, “ionizable” carboxyl groups may form a radical bearing a negative charge (—COO⁻), “ionizable” amino groups may form a radical bearing a positive charge (—NH₃⁺). A molecule bearing at the same time one or more ionizable acidic groups and one or more ionizable basic groups may form a zwitterion.

The term “reverse-phase chromatography” (RPC) includes any chromatographic method that uses a hydrophobic stationary phase and a polar mobile phase, which has a stronger affinity for hydrophobic or less polar molecules.

“Reverse-phase liquid chromatography” (RPLC) is a technique widely used in bio-pharmaceutical research and manufacturing. Commonly used stationary phase for RPLC is RP-modified silica gel, such as that with hydrocarbyl groups, such as alkyl chains and/or cycloalkyl or aryl groups (like phenyl, pentafluorophenyl, cyclohexyl) grafted on silica gel. “Monofunctional” alkylsilica stationary phases refer to those obtained from one molecule of silane reacting with one silanol group on the silica support. “Bidentate” bonded alkylsilica stationary phases refer to alkylsilica stationary phases obtained from one molecule of bidentate silane which contains two reactive groups reacting with two silanol group on the silica support. The bidentate stationary phases with two functional groups (e.g., alkyl groups like methyl, n-butyl, n-octyl or n-octadecyl) on one bidentate silane are also known as bifunctional stationary phases. Examples of alkylsilica stationary phase such as alkyl-bonded silica gel include but not limit to silica gel bonded with C18, C8, C4 or C1 alkyl, especially C18 alkyl. The alkyl in alkyl-bonded silica gel may be straight or branched. In an embodiment, the alkyl in alkyl-bonded silica gel is straight chain C18 alkyl (n-octadecyl). In an embodiment, the C18 alkyl in alkyl-bonded silica gel is branched and contains one or more types of side chain alkyl groups selected from C1-C6 alkyl (preferably isopropyl, isobutyl) and any combination thereof. Some alkylsilica stationary phase contains embedded functional groups such as carbamate group (e.g., Symmetry Shield™ RP C18), amide group (e.g., Zorbax Bonus -RP C18), etc. Mixed-mode modified silica gel are also used in RPLC, for example silica gel partially endcapped with hydrocarbyl groups and containing a certain proportion of residual (unendcapped) silica silanols, and silica gel partially endcapped with hydrocarbyl groups and containing a certain proportion of modified polar groups such as NH₂-modification. The RP-modified silica gel may be particles in any suitable shape, given that robust chromatographic separation can be achieved using suitable equipment. For example, alkyl-bonded silica gel may be generally spherical particles. In some cases, the RP-modified silica gel is porous on the surface, or is totally porous (surface and interior). In another embodiment, the RP-modified silica gel has a controlled surface porosity, with the pore size being 10 Å-1 µm, preferably 20-600 Å, more preferably 40-300 Å, for example 60 Å, 70 Å, 80 Å, 90 Å, 100 Å, 110 Å, 120 Å, 130 Å, 140 Å, 150 Å, 160 Å, 170 Å, 180 Å, 190 Å, 200 Å, 300 Å. In yet another embodiment, has a controlled surface porosity, with the pore size the RP-modified silica gel being.

The mobile phase of RPLC can be a mixed solvent of organic solvent and water. In some literatures the organic solvent is also referred to as “organic modifier”. Examples of aqueous binary mobile phase for RPLC include but not limited to ACN/H₂O, MeOH/H₂O, i-PrOH/H₂O or THF/H₂O. Examples of aqueous ternary mobile phase for RPLC include but not limited to THF/MeOH/H₂O.

The mechanism of RPLC generally relates to the following aspects: interaction between the stationary phase and solutes, which is controlled by changing the polarity of the mobile phase; solute-solvent and solvent-solvent dispersive interactions. The theory for solute retention in liquid chromatography has been developing since 1960s. Efforts are made to build retention models on the basis of molecular interactions such as solute-solvent interactions (Scott-Kucera interaction model), solute-solvent interactions and localization of solute and/or solvent during adsorption (Snyder-Soczewinski model, also known as linear solvent strength (LSS) model). However, given into consideration of the variability of the many factors and parameters influencing the chromatographic retention, the LC retention mechanisms are generally not known yet in details. On the development of “high-pressure chromatography”, by using small particle column packing and high column pressure, chromatographic precision and robustness are both improved, and some new retention modeling methods are reported, like solvatochromic analysis (an approach based on linear solvation-energy relationships, LSERs). The mechanism of RPLC for peptides is connected to the peptide size, and that for oligopeptides is similar to other small molecules. Some semi-empirical models have been proposed for the prediction of peptide retention, and some calculated “coefficients” are used to represent the interaction strength with which the amino acid residues interact with the stationary phase. These coefficients do not distinguish between hydrophobic, hydrophilic, ionic and other interactions. With the above developments, none of the prediction models performs satisfactorily in real experimental environments. Successful prediction of chromatographic behavior for massive identification of unknown small molecules or large molecules has not been reported.

“Hydrophobicity” is the association of nonpolar groups or molecules in an aqueous environment which arises from the tendency of water to exclude nonpolar molecules, and is understood as a measure of the relative tendency of a solute to prefer a nonaqueous over an aqueous environment or as a measure of the tendency of two (or more) solute molecules to aggregate in aqueous solutions. “Lipophilicity” represents the affinity of a molecule or a moiety for a lipophilic environment. It is commonly measured by its distribution behavior in a biphasic system, either liquid-liquid (e.g., partition coefficient in 1-octanol/water) or solid-liquid (e.g., retention on TLC or RP HPLC) systems. When used herein to describe a specific chemical entity, the terms “lipophilicity” and “hydrophobicity” is to stand for the same feature of the chemical entity, and therefore are used interchangeably.

The retention factor (k) of a component may be determined from the chromatogram using the formula k = (t_{R} - t_{M})/t_{M}= (V_{R} - V_{M})/V_{M}, wherein t_{R} and V_{R} are the retention time and retention volume of the component, respectively, t_{M} is the retention time of a nonretained component, such as an air or unretained solvent peak or unretained marker, V_{M} is the volume of mobile phase required for elution of an unretained component. The logarithmic form of retention factor, log k, is used as a lipophilicity index. However, for quantifying the lipophilicity, log k is not an equally important parameter as log P. Although in some cases log k shows good correlation with the shake-flask partition data (log P), dissimilarities between the slow equilibrium and chromatographic partition systems are evident.

Resolution (Rₛ) of the chromatography is calculated using the following formula: Rₛ = 2(t_{R2} - t_{R1})/(W₁ + W₂), where t_{R2} and t_{R1} are the retention times of the two components; and W₂ and W₁ are the corresponding widths at the bases of the peaks obtained by extrapolating the relatively straight sides of the peaks to the baseline.

When used herein, the expression “satisfactory chromatographic separation” and the similar expressions refer to that the Resolution (R_{S}) of the chromatography > 0.8, preferably > 0.9, for example, > 1.0, > 1.1, > 1.2, > 1.3, > 1.4, > 1.5, > 1.6, > 1.7, > 1.8, > 1.9 or> 2.0. In a particular embodiment, R_{S} > 1.0. In another particular embodiment, R_{S} > 1.5. A person skilled in the art can set and adjust expectations for the result of the chromatography according to different demands and situations. It is to be understood that in some circumstances, the purification need not to be thorough (100% purity), and a given purity (such as > 99%, > 98%, > 95%, > 90%, > 85%, > 80%, > 75%, > 70%, > 65% or > 60%) which is set forth before the separation may be regarded as satisfactory.

In RPLC for ionizable solutes, the retention time is highest for nonionized form and the lowest for ionized form of solutes. On changing of the mobile phase pH (for example in eluotropic series), retention time of an ionizable solute will always be within the retention time of its ionized and nonionized forms.

Eluotropic gradient in RPLC generally refers to a gradient wherein the content of organic solvent increases or wherein the pH value changes over time. The peak compression phenomenon features gradient separation. The movements of solutes located in the front part (closer to the column outlet) and back part (closer to column inlet) of the peak differ during the gradient separation. When solutes in the back part of the peak move faster than those in the front part, the compression of the peak width is observed. Therefore, by adjusting the content of organic solvent/pH value of the mobile phase, not only the retention time but also the peak width may be adjusted. However, it is noted that the change in peak width due to peak compression phenomenon in pH gradient separation cannot be precisely predicted.

pH gradient in the mobile phase is the outcome of parameters including for example the attribute of the solute(s), the content of the acidic/basic agent(s), the temperature, and the proportion of the organic solvent(s) when the mobile phase is partially aqueous.

The specific pKa values or range of pKa value mentioned in the present disclosure intends to mean those measured in aqueous solution. For pKa values unrevealed in the present disclosure, reference can be made to those published by IUPAC, for example, Ionisation Constants of Organic Acids in Aqueous Solution, Serjeant, E.P., Dempsey B., IUPAC Chemical Data Series No. 23, 1979. New York, New York: Pergamon Press, Inc., p. 989. The specific pH values or range of pH value intends to mean apparent pH values when the liquid or solution which is referred to is only partially aqueous.

Chromatographic methods under different ranges of pressure, such as atmospheric-pressure chromatography, medium-pressure chromatography and high-pressure chromatography are exploited in the industry. The term “overpressure chromatography” refers to chromatographic methods applying a pressure over the ambient pressure. “High-pressure chromatography” refers to chromatographic methods wherein the mobile phase and a liquid or an appropriately dissolved solid sample is forced through a column at high pressure, wherein the “high pressure” is sufficient to push the mobile phase at a desired flow speed through the particles of the stationary phase, which usually have a smaller particle size than those applied in atmospheric-pressure chromatography or medium-pressure chromatography. In an embodiment, the stationary phase used in the RPLC method according to the present disclosure is a RP-modified silica gel with the particle size being 1-300 µm, preferably 1-200 µm, more preferably 1-100 µm, further preferably 1-80 µm, more preferably 3-60 µm, for example 3 µm, 4 µm, 5 µm, 6 µm, 7 µm, 8 µm, 9 µm, 10 µm, 11 um, 12 µm, 13 µm, 14 µm, 15 µm, 20 µm, 40 µm, 50 µm, 60 µm.

Reversed-phase ion-pairing (RPIP) chromatography (RPIPc) (also known as ion-pairing reversed-phase chromatography (IPRP)) refers to chromatographic methods wherein small amounts of ion pairs are added (e.g., by adding an ion-pairing reagent) to the mobile phase which results in increasing retention of strongly polar compounds. The retention of the resulting ion pair is controlled by pH, counterion concentration and mobile phase polarity. In some cases, the strongly polar compound is a charged molecule which would not be retained in regular reversed-phase chromatography. The mechanism of RPLC does not exclude possible influences of ion pairs. The presence of acidic or basic agent in the RPLC mobile phase may influence the chromatographic behavior of the solute in the respect of the pH value of the environment and also possibly through the formation of ion pairs with the solutes, for example with ionized form of the solutes.

The term “chromatographic process” is an abstract term and generally refers to the separation process using a chromatographic method. A separation process can comprise operations for separation, and optionally the operations for cleansing, balancing, or regenerating the chromatographic media.

The “separation range” of the eluent refers to the eluent used in separation, and the “balancing range” of the eluent refers to the eluent used for balancing. It is to be understood that a separation range could mean an eluent composition which changes with the elapse of time, and the change could be terminated by the termination of the time period, for example due to a critical incident such as the completion of the elution of the aimed product.

### The Processes According to the Disclosure

### Process for Separation

In a first aspect of the disclosure, provided is a process for separating one or more target compounds from Mixture 1 comprising four compounds, each of the four compounds comprising Moiety 1, Moiety 2 and Moiety 3;

wherein Moiety 1 has a ring-opened thiosuccinimide structure selected from formulae (I) to (IV):

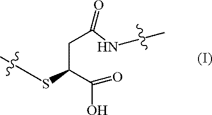

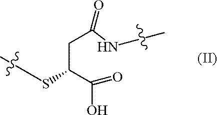

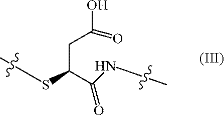

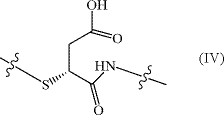

wherein
the thiol group and the amido group in Moiety 1 constitutes two linking sites, through one of which Moiety 2 is linked to Moiety 1, and through the other of which Moiety 3 is linked to Moiety 1;
   Moiety 2 contains one or more chiral centers;
   Moiety 1 in the four compounds is different;
   Moiety 3 is the rest moiety of the molecule;
   the weight of Moiety 3 is no more than 1900;
the one or more target compounds are selected from the said four compounds comprised by Mixture 1;
the process comprising the following steps (1) and (2)
   (1) providing Mixture 1;
   (2) subjecting Mixture 1 to chromatography so as to obtain the target compound(s), wherein
      a. each of the target compound(s) are obtained in a separate product, or
      b. two target compounds are obtained in a mixture, which is defined as Mixture 2.

Formulae (I) and (II) are referred to as β, and the configuration of formulae (III) and (IV) are referred to as α, according to the position of the thiol group.

In one embodiment, the process further comprises the steps (3) and (4)
(3) recovering an eluate collected in step (2) comprising Mixture 3, wherein Mixture comprises one or more additional target compounds which are different from those separated in step (2);
(4) subjecting the eluate recovered in step (3) to chromatography to separate the additional target compound(s).

In one embodiment, the step (1) is optional. In another embodiment, Mixture 1 is provided by synthesis method known in the art.

In one embodiment, each of the one or more target compounds are in predominantly, e.g., in pure, or substantially pure isomeric form, e.g., substantially free of other isomeric forms, e.g., having a purity of greater than 90%, e.g., greater than 95%, e.g., greater than 98%, e.g., a least 99%.

### 1. Chromatographic Steps

In one embodiment, Moiety 1 in each target compound is different.

In one embodiment, in step (2) and step (4), four target compounds are obtained, wherein two target compounds are obtained in separate products in step (2) and the other two target compounds are obtained in separate products in step (4).

In a preferable embodiment, Moiety 1 in the two target compounds obtained in separate products in step (2) each have the structure of formula (I) or (II); and Moiety 1 in the two target compounds obtained in separate products in step (4) each have the structure of formula (III) or (IV).

In another preferable embodiment, three target compounds comprised by Mixture 1 are obtained in separate products in step (2) and the remaining one is obtained in step (4).

In yet another preferable embodiment, in step (2), four target compounds are obtained, wherein two target compounds are obtained in separate products and the remaining two target compounds are obtained in Mixture 2. In a more preferable embodiment, Moiety 1 in the two target compounds obtained in separate products in step (2) each have the structure of formula (I) or (II); and Moiety 1 in the two target compounds obtained in Mixture 2 each have the structure of formula (III) or (IV).

### 2. Stationary Phase

In one embodiment, the chromatography in step (2) and step (4) is reverse-phase chromatography. In another embodiment, the stationary phases used in the reverse-phase chromatography in step (2) and step (4) are each independently selected from species of alkyl-bonded silica gel.

The alkyl-bonded silica gel applied in the present disclosure is not particularly limited. Any alkylsilica stationary phase may be used as long as an acceptable chromatographic result could be achieved. In a preferable embodiment, the stationary phases used in the reverse-phase chromatography in step (2) and step (4) are C18-bonded silica gel (i.e. C18 alkyl-bonded silica gel). In a preferable embodiment, the alkyl in C18-bonded silica gel is straight chain C18 alkyl (n-octadecyl). In one embodiment, the C18-bonded silica gel is generally spherical particles. In another embodiment, the C18-bonded silica gel has a controlled surface porosity, with the pore size being 20-600 Å, preferably 40-300 Å, more preferably 40-200 Å, for example 60 Å, 100 Å, 110 Å, 120 Å, 150 Å, 200 Å, 300 Å.

### 3. Mobile Phase

In one embodiment, the mobile phase in step (2) are as follows:
Eluent composition:
Eluent A: Water optionally containing Acidic agent 1;
Eluent B: Organic solvent 1 optionally containing Acidic agent 2;
provided that at least one of Acidic agent 1 and Acidic agent 2 is present;
separation range: B in gradient from about 0%-30% to about 30%-100%, with the remainder being A;
wherein the Acidic agents 1 and 2 are independently an inorganic acid or an organic acid.

In one embodiment, Organic solvent 1 is selected from methanol and ACN. In a preferable embodiment, Organic solvent 1 is methanol.

In one embodiment, separation range of the mobile phase in step (2) is as follows: B in gradient from about 40%-50% to about 50%-70%, with the remainder being A.

In one embodiment, at least one of Acidic agent 1 and Acidic agent 2 is present. In a preferable embodiment, both Acidic agent 1 and Acidic agent 2 are present.

In one embodiment, the amount of Acidic agent 1 and Acidic agent 2 (if present) are such that the gradient of eluent C and eluent D is accompanied by a gradient of Acidic agent 1 and Acidic agent 2.

In one embodiment, the Acidic agent 1 has the structure of R^{c}(COOH)_{d}; wherein d is 1 or 2, R^{c} is C₁₋₁₅ hydrocarbyl or hydrocarbylene, optionally substituted by at least one substituents selected from R^{y}, wherein R^{y} is selected from —OH, —OCH₃, —OCH₂CH₃, —SH, —SCH₃, preferably —OH. In a preferable embodiment, the C₁₋₁₅ hydrocarbyl in R^{c} is C₁₋₁₅ alkyl, alkylene, C₁₋₁₅ alkenyl or C₁₋₁₅ alkenylene, optionally substituted by one, two, three or four substituents selected from R^{y}. In another preferable embodiment, d is 1, and the C₁₋₁₅ hydrocarbyl in R^{c} is C₁₋₁₀ alkyl, preferably C₁₋₄ alkyl. In a preferable embodiment, the C₁₋₁₅ hydrocarbyl in R^{c} is C₁₋₂ alkyl. In a particular embodiment, the C₁₋₁₅ hydrocarbyl in R^{C} is methyl. In a preferable embodiment, d is 2, and the C₁₋₁₅ hydrocarbyl in R^{c} is C₂₋₄ alkylene, especially ethylene, optionally substituted by one or two -OH groups.

In one embodiment, the Acidic agent 1 is selected from organic acids. In one embodiment, the pKa value of Acidic agent 1 is from 0.1 to 6.5, for example 0.4 to 6.5, 0.6 to 6.5, 1.0 to 6.5, 2.0 to 6.5 or 2.0 to 5.5. In a preferable embodiment, the Acidic agent 1 is selected from organic acids, and the pKa value of Acidic agent 1 is from 4.0 to 5.5, for example about 4.0, about 4.1, about 4.2, about 4.3, about 4.4, about 4.5, about 4.6, about 4.7, about 4.8, about 4.9, about 5.0, about 5.1, about 5.2, about 5.3, about 5.4 or about 5.5, especially about 4.7 or about 4.8, for example about 4.71, about 4.72, about 4.73, about 4.74, about 4.75, about 4.76, about 4.77, about 4.78 or about 4.79. In another preferable embodiment, the Acidic agent 1 is selected from organic acids, and the pKa value of Acidic agent 1 is from 2.3 to 3.8, for example about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, or about 3.8, especially about 3.0 or about 3.1, for example about 3.01, about 3.02, about 3.03, about 3.04, about 3.05, about 3.06, about 3.07, about 3.08 or about 3.09.

In another embodiment, the Acidic agent 1 is selected from AcOH and L-tartaric acid (L-TA). In a preferable embodiment, Acidic agent 1 is AcOH. In one embodiment, the amount of Acidic agent 1 is about 0.01% to about 1%, based on the total volume of eluent A. In a preferable embodiment, the amount of Acidic agent 1 is about 0.05% to about 0.5%, preferably about 0.1% to about 0.5%, more preferably about 0.1% to about 0.3%, especially 0.3%, based on the total volume of eluent A.

In one embodiment, Acidic agent 2 is not present. In another embodiment, the species of Acidic agent 2 is the same with Acidic agent 1.

In one embodiment, the amount of Acidic agent 2 is about 0.01% to about 1%, based on the total volume of eluent B. In another embodiment, the amount of Acidic agent 2 is about 0.05% to about 0.5%, preferably about 0.1% to about 0.5%, more preferably about 0.1% to about 0.3%, especially 0.3%, based on the total volume of eluent B.

In one embodiment, when a target compound is being eluted, the total amount of Acidic agent 1 and Acidic agent 2 is no more than about 1%, preferably no more than about 0.8%, more preferably no more than about 0.6%, for example, no more than about 0.4%, especially no more than about 0.285%, based on the total volume of eluent A and eluent B.

In one embodiment, the total contents of Acidic agent 1 and Acidic agent 2 (if present) is about 0.005 to 0.06 M in the eluotropic composition.

In an alternative embodiment, the contents of Acidic agent 1 and Acidic agent 2 are set forth and/or adjusted dynamically to obtain an aimed pH profile, for example, to obtain an aimed isocratic pH value or an aimed pH gradient.

In one embodiment, the pH of the mobile phase is from about 1.0 to about 4.0. In another embodiment, the pH of the mobile phase is from about 1.0 to about 3.5. In one embodiment, the pH of the mobile phase is from about 2.0 to about 3.5. In another embodiment, the pH of the mobile phase is from about 2.4 to about 3.2.

In one embodiment, the mobile phase in step (4) are as follows:
Eluent composition:
Eluent C: Water optionally containing Acidic agent 3;
Eluent D: Organic solvent 2 optionally containing Acidic agent 4;
separation range: D in gradient from about 0%-30% to about 30%-100%, with the remainder being C;
wherein the Acidic agents 3 and 4 are independently an inorganic acid or an organic acid.

In one embodiment, Organic solvent 2 is selected from methanol and ACN. In a preferable embodiment, Organic solvent 2 is ACN.

In one embodiment, at least one of Acidic agent 3 and Acidic agent 4 is present. In a preferable embodiment, both Acidic agent 3 and Acidic agent 4 are present.

In one embodiment, separation range of the mobile phase in step (4) is as follows: D in gradient from about 10%-30% to about 30%-95%, with the remainder being C.

In one embodiment, the amount of Acidic agent 3 and Acidic agent 4 (if present) are such that the gradient of eluent C and eluent D is accompanied by a gradient of Acidic agent 3 and Acidic agent 4.

Species of Acidic agent 3 and Acidic agent 4 are not particularly limited, as long as satisfactory chromatographic separation can be achieved. In one embodiment, the Acidic agent 2 is selected from organic acids. In another embodiment, the pKa value of Acidic agent 2 is from 0.1 to 6.5, for example about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, or about 6.5.

In one embodiment, Acidic agent 3 and Acidic agent 4 are independently selected from TFA, phosphate buffer, ammonium acetate (AA), AcOH, H₃PO₄, TEAP and L-tartaric acid (L-TA). In a preferable embodiment, Acidic agent 3 is selected from TFA, phosphate buffer, AA and TEAP. In a more preferable embodiment, Acidic agent 3 is selected from TFA, phosphate buffer and TEAP, especially TFA.

In one embodiment, Acidic agent 4 is not present. In another embodiment, the species of Acidic agent 4 is the same with Acidic agent 3.

In one embodiment, the amount of Acidic agent 3 is about 0.01% to about 1%, based on the total volume of eluent C. In another embodiment, the amount of Acidic agent 3 is about 0.05% to about 0.5%, preferably about 0.05% to about 0.3%, especially 0.1%, based on the total volume of eluent C.

In one embodiment, the amount of Acidic agent 4 is about 0.01% to about 1%, based on the total volume of eluent D. In another embodiment, the amount of Acidic agent 4 is about 0.05% to about 0.5%, preferably about 0.05% to about 0.3%, especially 0.1%, based on the total volume of eluent D.

In one embodiment, the separation range of the mobile phase in step (4) are as follows: D in gradient from about 10%-30% to about 30%-95%, with the remainder being C.

In one embodiment, the total contents of Acidic agent 3 and Acidic agent 4 (if present) is about 0.01 to 0.25 M in the eluotropic composition.

In an alternative embodiment, the contents of Acidic agent 3 and Acidic agent 4 are set forth and/or adjusted dynamically to obtain an aimed pH profile, for example, to obtain an aimed isocratic pH value or an aimed pH gradient.

In one embodiment, the pH of the mobile phase is from about 1.0 to about 6.0. In a specific embodiment, the pH of the mobile phase is from about 2.0 to about 5.0, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0.

In one embodiment, an isocratic pH value is applied, and the isocratic pH value is in the range of about 2.0 to about 6.0. In another embodiment, the isocratic pH value of the mobile phase is about 2.0, about 2.5, about 3.0, about 3.5, about 4.0, about 5.0 or about 6.0.

In an alternative embodiment, the process further comprises an optional balancing step before step (2), the mobile phase is as defined above, and the balancing range of the mobile phase is about 100%-95% A and about 0%-5% B, for example about 95% A and about 5% B. In another alternative embodiment, the process further comprises an optional balancing step before step (4), the mobile phase is as defined above, and the balancing range of the mobile phase is about 100%-95% C and about 0%-5% D, for example about 95% C and about 5% D.

### 4. Solute (Target Compounds)

In one embodiment, the log P (octanol-water Partition coefficient) value of Moiety 2 is from about 1 to about 5; preferably from about 1.5 to about 4.5; from about 2 to about 4.5; or from about 2.5 to about 4.5, for example about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, or about 4.5.

In one embodiment, Moiety 2 has the structure of the following Formula (V):

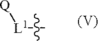

wherein Q is a group comprising at least one chiral centers; andL¹ is not present, or is a bivalent group being one or more selected from the group consisting of C₁₋₁₀ alkylene, C₃₋₁₀ cycloalkylene, C₆-₁₀ arylene, 4 to 10 membered heterocyclylene, 5 to 10 membered heteroarylene, —NH—, —(CO)—, —NH(CO)— and —(CO)NH—.

In one embodiment, Q is a C₅-₅₀ hydrocarbyl, wherein
one or more “CH₂” structures in the hydrocarbyl are optionally replaced by —O—, —S—, —NH—, —C(═O)—, —S(═O)—, —S(═O)₂—, —NH(C═O)—, —C(═O)NH—, —NH—, —S(═O)—, —S(═O)NH—, —NHS(═O)₂— or —S(═O)₂NH—, given that a stable structure is formed;
one or more “CH” structures in the hydrocarbyl are optionally replaced by N or P, given that a stable structure is formed;
one or more carbon atoms, sulfur atoms, nitrogen atoms or phosphorus in Q are independently and optionally substituted by oxo (═O);
Q is optionally substituted by at least one substituents selected from R^{q}, wherein each R^{q} is independently selected from R^{a1}, -OR^{a1}, -SR^{a1}, -NR^{a1}R^{b1}, -C(=O)OR^{a1}, -C(=O)NR^{a1}R^{b1}, -C(=O)R^{a1}, -S(=O)₂OR^{a1}, -S(=O)₂R^{a1}, -S(=O)₂NR^{a1}R^{b1}, -S(=O)R^{a1}, -C(=S)OR^{a1}, -C(=S)NR^{a1}R^{b1}, -C(=S)R^{a1}, -P(=O)(OR^{a1})OR^{b1}, -C(=NR^{a1})NR^{b1}R^{c1}; and
R^{a1}, R^{b1} and R^{c1} are each independently selected from hydrogen and C₁₋₆ alkyl.

In one embodiment, each cycloalkyl structure in Q is independently and optionally replaced by heterocyclic structures with the same radical valence and the same number of ring atom number with the cycloalkyl structure.

In one embodiment, each aryl structure in Q is independently and optionally replaced by heteroaryl structures with the same radical valence and the same number of ring atom number with the aryl structure.

In one embodiment, each R^{q} is independently selected from halogen, -C₁₋₃ alkyl, —OH, -O-C₁₋₃ alkyl, —SH, -S-C₁₋₃ alkyl, - C(=O)-C₁₋₃ alkyl and -S(=O)₂-C₁₋₃ alkyl. In a very specific embodiment, R^{q} is selected from halogen, —CH₃, —OH and —OCH₃.

In one embodiment, L¹ is not present.

In one embodiment, Moiety 2 has the structure of the following Formula (V-1):

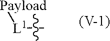

wherein Payload is selected from the group consisting of hydrogen and small molecule compounds comprising at least one chiral centers; and
L¹ is as defined above.

In one embodiment, the small molecule is selected from enzyme inhibitors, enzyme activators, receptor modulators (such as agonists or antagonists), toxins (such as cytotoxins), glycans, PEG moieties, radionuclides (e.g., ²²⁵Ac, ²¹¹At, ^{212Bi}, ²¹³Bi, ⁶⁷Ga, ¹²³I, ¹²⁴I, ¹²⁵I, ¹³¹I, ¹¹¹In, ¹⁷⁷Lu, ^{191m}Os, ^{195m}Pt, ¹⁸⁶Re, ¹⁸⁸Re, ¹¹⁹Sb, ¹⁵³Sm, ^{99m}Tc, ²²⁷Th and ⁹⁰Y), nucleic acids and analogues (e.g., interfering RNAs), tracer molecules (e.g., fluorophores and fluorescent molecules), low molecular weight peptides (e.g., protein tags, bioactive peptides, protein toxins and enzymes with a molecular weight below 2000 Da, below 1000 Da, below 900 Da, below 800 Da, below 700 Da, below 600 Da, or below 500 Da), low molecular weight peptidomimetics, low molecular weight antibodies (e.g., nanobodies) and antibody fragments.

In one embodiment, Moiety 2 does not contain ionizable acidic groups or ionizable basic groups. In another embodiment, Moiety 2 contains a certain number of ionizable acidic groups and ionizable basic groups, and under the chromatographic conditions used herein, the total charge of Moiety 2 is approximately zero. The ionizable acidic groups include but not limit to carboxyl groups, sulfinic acid groups, sulfonic acid groups, phosphinic acid groups and phosphonic acid groups, etc, especially carboxyl groups. The ionizable basic groups include but not limit to amino groups, amine groups, etc., especially amino groups. In an alternative embodiment, Payload does not contain ionizable carboxyl groups or ionizable amino groups. In another alternative embodiment, Payload contains an equal number of ionizable carboxyl groups and ionizable amino groups.

In one embodiment, Moiety 2 has the structure of the following Formula (V-1-1):

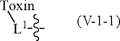

wherein Toxin is a cytotoxin moiety containing one or more chiral centers, and L¹ is as defined above.

In one embodiment, L¹ is not present, or is selected from C₁₋₁₀ alkylene, wherein one or more (—CH₂—) structures in the alkyl is optionally substituted by oxo (═O).

In one embodiment, the cytotoxin is selected from the group consisting of taxanes, maytansinoids, auristatins, epothilones, combretastatin A-4 phosphate, combretastatin A-4 and derivatives thereof, indol-sulfonamides, vinblastines such as vinblastine, vincristine, vindesine, vinorelbine, vinflunine, vinglycinate, anhy-drovinblastine, dolastatin 10 and analogues, halichondrin B and eribulin, indole-3-oxoacetamide, podophyllotoxins, 7-diethylamino-3-(2′-benzoxazolyl)-coumarin (DBC), discodermolide, laulimalide. In another embodiment, the cytotoxin is selected from the group consisting of DNA topoisomerase inhibitors such as camptothecins and derivatives thereof, mitoxantrone, mitoguazone. In a preferable embodiment, the cytotoxin is selected from the group consisting of nitrogen mustards such as chlorambucil, chlornaphazine, cholophosphamide, estramustine, ifosfamide, mechlorethamine, mechlorethamine oxide hydrochloride, melphalan, novembichin, phenamet, phenesterine, prednimustine, trofosfamide, uracil mustard. In yet another preferable embodiment, the cytotoxin is selected from the group consisting of nitrosoureas such as carmustine, flubenzuron, formoterol, lomustine, nimustine, ramustine. In one embodiment, the cytotoxin is selected from the group consisting of aziridines. In a preferable embodiment, the cytotoxin is selected from the group consisting of benzodopa, carboquone, meturedepa, and uredepa. In one embodiment, the cytotoxin is selected from the group consisting of an anti-tumor antibiotic. In a preferable embodiment, the cytotoxin is selected from the group consisting of enediyne antibiotics. In a more preferable embodiment, the cytotoxin is selected from the group consisting of dynemicin, esperamicin, neocarzinostatin, and aclacinomycin. In another preferable embodiment, the cytotoxin is selected from the group consisting of actinomycin, antramycin, bleomycins, actinomycin C, carabicin, carminomycin, and cardinophyllin, carminomycin, actinomycin D, daunorubicin, detorubicin, adriamycin, epirubicin, esorubicin, idarubicin, marcellomycin, mitomycins, nogalamycin, olivomycin, peplomycin, porfiromycin, puromycin, ferric adriamycin, rodorubicin, rufocromomycin, streptozocin, zinostatin, zorubicin. In yet another preferable embodiment, the cytotoxin is selected from the group consisting of trichothecene. In a more preferable embodiment, the cytotoxin is selected from the group consisting of T-2 toxin, verracurin A, bacillocporin A, and anguidine. In one embodiment, the cytotoxin is selected from the group consisting of an anti-tumor amino acid derivatives. In a preferable embodiment, the cytotoxin is selected from the group consisting of ubenimex, azaserine, 6-diazo-5-oxo-L-norleucine. In another embodiment, the cytotoxin is selected from the group consisting of folic acid analogues. In a preferable embodiment, the cytotoxin is selected from the group consisting of dimethyl folic acid, methotrexate, pteropterin, trimetrexate, and edatrexate. In one embodiment, the cytotoxin is selected from the group consisting of purine analogues. In a preferable embodiment, the cytotoxin is selected from the group consisting of fludarabine, 6-mercaptopurine, tiamiprine, thioguanine. In yet another embodiment, the cytotoxin is selected from pyrimidine analogues. In a preferable embodiment, the cytotoxin is selected from the group consisting of ancitabine, gemcitabine, enocitabine, azacitidine, 6-azauridine, carmofur, cytarabine, dideoxyuridine, doxifluridine, floxuridine. In one embodiment, the cytotoxin is selected from the group consisting of androgens. In a preferable embodiment, the cytotoxin is selected from the group consisting of calusterone, dromostanolone propionate, epitiostanol, mepitiostane, testolactone. In another embodiment, the cytotoxin is selected from the group consisting of anti-adrenals. In a preferable embodiment, the cytotoxin is selected from the group consisting of aminoglutethimide, mitotane, and trilostane. In one embodiment, the cytotoxin is selected from the group consisting of anti-androgens. In a preferable embodiment, the cytotoxin is selected from the group consisting of flutamide, nilutamide, bicalutamide, leuprorelin acetate, and goserelin. In yet another embodiment, the cytotoxin is selected from the group consisting of a protein kinase inhibitor and a proteasome inhibitor.

In a particular embodiment, the cytotoxin is selected from the group consisting of vinblastines, colchicines, taxanes, auristatins, and maytansinoids. In another particular embodiment, the cytotoxin is an maytansinoid, such as DM1 and the like. Note that where a cytotoxin comprising a thiol moiety is used, the thiol moiety being capable of reaction with a maleimide moiety to form a thiosuccinimide, for example a maytansinoid, e.g., DM1, a linker (L¹) is not required, as the cytotoxin can link directly via the thiosuccinimide. In such case, it could be understood that in some embodiments Payload and the thiol moiety together constitutes a cytotoxin, and therefore in such case Payload is the rest moiety of the cytotoxin molecule except for the thiol moiety.

In one embodiment, each Moiety 3 of the four compounds in Mixture 1 are identical, and the molecular weight of Moiety 3 is no more than 1900 Da.

In one embodiment, the molecular weight of Moiety 3 is no more than 1800 Da; no more than 1700 Da; preferably no more than 1600 Da; no more than 1500 Da; no more than 1400 Da; no more than 1300 Da; no more than 1200 Da; no more than 1100 Da; no more than 1000 Da; no more than 900 Da; no more than 800 Da; no more than 700 Da; no more than 600 Da; no more than 500 Da; no more than 400 Da; no more than 300 Da; or no more than 200 Da. In a particular embodiment, the molecular weight of Moiety 3 is from about 100 to about 2000 Da; preferably from about 100 to about 1500 Da; from about 100 to about 1000 Da; from about 200 to about 1000 Da; or from about 200 to about 600 Da.

In a particular embodiment, Moiety 3 has the structure of the following Formula (VI):

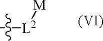

wherein L² is a bond, or is a bivalent group being one or more selected from the group consisting of C₁₋₁₀ alkylene, C₃₋₁₀ cycloalkylene, C₆-₁₀ arylene, 4 to 10 membered heterocyclylene, 5 to 10 membered heteroarylene, —NH—, —(CO)—, —NH(CO)— and —(CO)NH—;
M comprises: (1) Amino acid sequence 1 comprising 1-20 amino acids, (2) an optional polyethylene glycol (PEG) moiety, which comprises a “—(C₂H₄—O)ᵢ—” structure, i being an integer of 1 to 100, and (3) an optional group Y, which is bivalent and is selected from the group consisting of Cleavable sequence 1, spacer Sp1, and the combination thereof;
the cleavable sequence comprises Amino acid sequence 2 which can be cleaved by enzyme, and the cleavable sequence comprises 1-10 amino acids;
Sp1 is selected from the group consisting of a spacer sequence containing 1-20 amino acids, PAB, and the combination thereof.

In one embodiment, Amino acid sequence 1 comprises a ligase recognition motif (i.e., the recognition motif of the ligase donor substrate, or the recognition motif of the ligase acceptor substrate. In one embodiment, the ligase is a transpeptidase. In a preferred embodiment, the ligase is a sortase. In one embodiment, the sortase is selected from the group consisting of sortase A (SrtA), sortase B (SrtB), sortase C (SrtC), sortase D (SrtD), sortase E (SrtE), sortase F (SrtF) and a combination thereof. In one embodiment, the ligase is a SrtA. In another particular embodiment, the ligase recognition motif is selected from LPXTG, wherein X can be any single amino acid that is natural or unnatural. In a particular embodiment, the ligase recognition motif is LPETG. In yet another particular embodiment, the ligase recognition motif is Gₙ, wherein G is glycine (Gly), and n is an integer of 3 to 10.

In one embodiment, i is 1 to 10.

In one embodiment, Y is a bond, or is selected from the group consisting of a cleavable sequence, spacer Sp1, and the combination thereof. In a particular embodiment, Y is a bond. In one embodiment, Amino acid sequence 2 can be recognized as enzyme substrate and can be cleaved by the enzyme. In a particular embodiment, Amino acid sequence 2 can be enzymatically cleaved in the lysosomal of the cell. In another particular embodiment, Amino acid sequence 2 can be cleaved by protease, in particular by cathepsins. In yet another particular embodiment, Amino acid sequence 2 can be cleaved by glutaminase. In one embodiment, Amino acid sequence 2 is selected from the group consisting of a cathepsin restriction site, a glutaminase restriction site, and combinations thereof. In one embodiment, the cleavable sequence is selected from Phe-Lys, Val-Cit, Val-Lys, Gly-Phe-Leu-Gly, Ala-Leu-Ala-Leu and the combination thereof.

In one embodiment, Y is a bond, or is selected from spacer Sp1. In another embodiment, Sp1 is a spacer sequence comprising 1-10, preferably 1-6, more preferably 1-4 amino acids. In a particular embodiment, Sp1 is Leu. In another particular embodiment, Sp1 is Gln. In one embodiment, Sp1 is PAB. In yet another embodiment, Y is selected from the group consisting of Phe-Lys-PAB, Val-Cit-PAB, and Val-Lys-PAB.

In one embodiment, the amino acids comprised by Y may be natural or unnatural. In a particular embodiment, Y is a bond, or is Amino acid sequence 3. Amino acid sequence 3 comprises 1-30 natural or unnatural amino acids, which are each independently the same or different. And Amino acid sequence 3 is selected from the group consisting of: Cleavable sequence 1 comprising 1-10 amino acids, spacer Sp1 comprising 1-20 amino acids, and the combination thereof.

In one embodiment, M is selected from the group consisting of lysine, oligomeric glycine, oligomeric alanine, a mixture of oligomeric glycine/alanine having a degree of polymerization of 3-10, and the combination thereof.

In one embodiment, the pKa of Moiety 3 is more than 7 and less than 12. In a preferable embodiment, the pKa of Moiety 3 is from about 8 to about 12, preferably about 9 to about 11, for example about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, especially about 10.0.

In a particular embodiment, M is L(G)ₙ, wherein G is glycine (Gly), and n is an integer of 3 to 10, especially 3. In another particular embodiment, M is Gₙ. In one embodiment, C-terminal of M is connected to L², and M has an ionizable amino group. In a particular embodiment, M has only one ionizable group, which is an ionizable amino group. The ionizable amino group can be ionized to the radical —NH₃⁺. The ionized form —NH₃⁺ of the amino group may optionally interact with the solvent molecule or a chemical entity, the chemical entity bearing a negative charge (+) or a partial negative charge (δ+).

In an alternative embodiment, M is LPXTGJ, wherein X can be any single amino acid that is natural or unnatural; J is absent, or is an amino acid fragment comprising 1-10 amino acids, optionally labeled. In one embodiment, J is absent. In yet another embodiment, J is an amino acid fragment comprising 1-10 amino acids, wherein each amino acid is independently any natural or unnatural amino acid. In another embodiment, J is Gₘ, wherein m is an integer of 1 to 10. In yet another particular embodiment, M is LPETG. In another particular embodiment, M is LPETGG. In one embodiment, N-terminal of M is connected to L², M has a free C-terminal carboxyl group. In a particular embodiment, M has only one ionizable group, which is an ionizable carboxyl group. The ionizable carboxyl group can be ionized to the radical —COO⁻. The ionized form —COO⁻ of the carboxyl group may optionally interact with the solvent molecule or a chemical entity, the chemical entity bearing a positive charge or a partial positive charge (δ+).

In one embodiment, and the structure of Formula (VI) has a structure of the following Formula (VI-1)

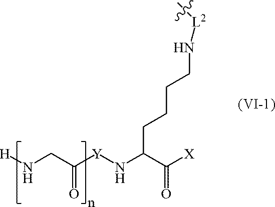

wherein n is an integer of 3 to 10, x is selected from the group consisting of hydrogen, OH, NH₂, an amino acid fragment comprising 1-10 amino acids, and a nucleotide fragment comprising 1-10 nucleotides.

In one embodiment, n is 3. In a preferred embodiment, x is selected from OH, NH₂, an amino acid fragment comprising 1-10 amino acids. In a more preferred embodiment, x is selected from OH, NH₂ and Gly. In a particular embodiment, x is NH₂.

In one embodiment, L¹ and L² are each independently selected from bond and C₁₋₁₀ alkyl, and one or more (—CH₂—) structures in the alkyl is optionally substituted by oxo (═O).

In one embodiment, L¹ in Moiety 2 is selected from

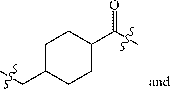

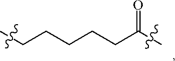

and L² in Moiety 3 is a bond.

In another embodiment, L¹ in Moiety 2 is a bond, and L² in Moiety 3 is selected from

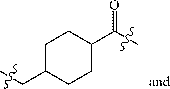

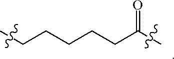

In one embodiment, the four compounds comprised by Mixture 1 each independently have a structure selected from the following Formulae (1) and (1′)

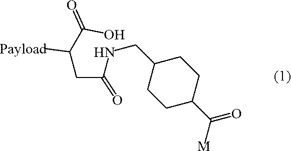

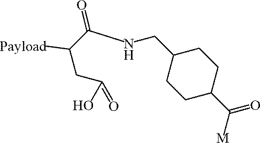

(1′) wherein Payload is as defined in Formula (V-1), and M is as defined in Formula (VI), respectively.

In one embodiment, the four compounds comprised by Mixture 1 each independently have a structure selected from the following Formulae (2) and (2′)

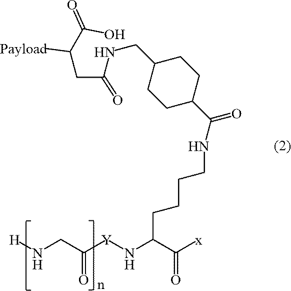

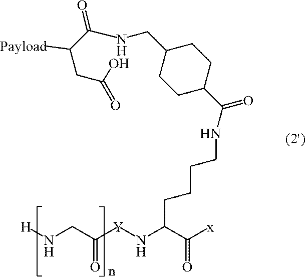

wherein Payload is as defined in Formula (V-1), Y is as defined in Formula (VI), and n and x are as defined in Formula (VI-1), respectively.

In a particular embodiment, the compounds of formulae (1) and (1′) are products from ring-opening reaction of the thiosuccinimide group in the following Formula (3);

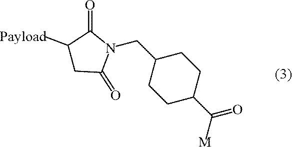

wherein Payload is as defined in Formulae (V-1), and M is as defined in Formula (VI), respectively.

In a particular embodiment, the compounds of formulae (2) and (2′) are products from ring-opening reaction of the thiosuccinimide group in the following Formula (4);

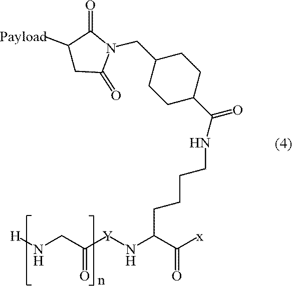

wherein Payload is as defined in Formulae (V-1), and n and x are as defined in Formula (VI-1), respectively.

The ring-opening reaction of the thiosuccinimide group may be conducted with any known method in the art. For example, method of ring-opening reaction can be found in WO2015165413A1.

In a particular embodiment, M has an ionizable amino group; and Acidic agent 1 is AcOH; and the amount of Acidic agent 1 is about 0.01% to about 1%, preferably about 0.05% to about 0.5%, more preferably about 0.1% to about 0.5%, more preferably about 0.1% to about 0.3%, especially 0.3%, based on the total volume of eluent A; and Acidic agent 2 is not present.

In another particular embodiment, M has an ionizable amino group; and Acidic agent 3 is TFA; and the amount of Acidic agent 3 is about 0.01% to about 1%, preferably about 0.05% to about 0.5%, more preferably about 0.05% to about 0.3%, especially 0.1%, based on the total volume of eluent C; and Acidic agent 4 is not present or presents in an amount of about 0.01% to about 1%, preferably about 0.05% to about 0.5%, more preferably about 0.05% to about 0.3%, especially 0.1%, based on the total volume of eluent D.

In one embodiment, Mixture 1 is the reaction mixture resulted from the ring-opening reaction of a thiosuccinimide group, which affords the ring-opened thiosuccinimide structure of Moiety 1.

In an alternative embodiment, M comprises one or more primary amino groups; and the Acidic agents 1 to 4 can respectively from ion pair with one or more primary amino groups comprised by M. In another alternative embodiment, the Acidic agents 1 to 4 are independently selected from AcOH, L-TA and TFA. In a very special embodiment, Acidic agent 1 is AcOH or L-TA, preferably AcOH. In another very special embodiment, Acidic agent 3 is TFA.

In an alternative embodiment, Moiety 1 is further substituted by R¹ and Moiety 1 has a structure selected from the following Formulae (VII) to (X):

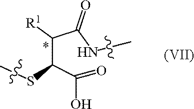

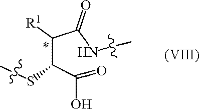

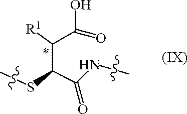

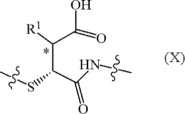

wherein R¹ is selected from hydrogen and C₁₋₁₀ alkyl, and the chiral configuration of each R¹ is identical in Formulae (VII) to (X).

In one embodiment, the chromatography in step (2) is conducted by reverse-phase HPLC. In an alternative embodiment, the chromatography in step (4) is conducted by a process selected from atmospheric-pressure chromatography, medium-pressure chromatography and high-pressure chromatography. In another embodiment, the reverse-phase chromatography in step (4) is conducted by reverse-phase HPLC.

In a particular embodiment, the four compounds comprised by Mixture 1 has the structure of formulae (i) to (iv), respectively.

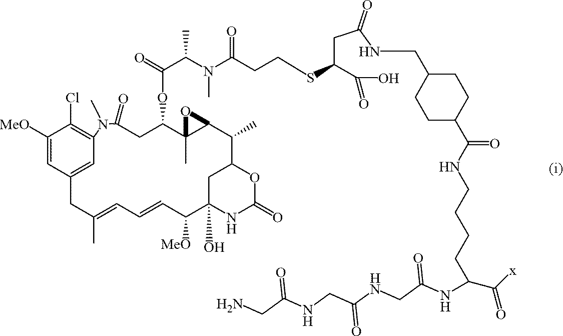

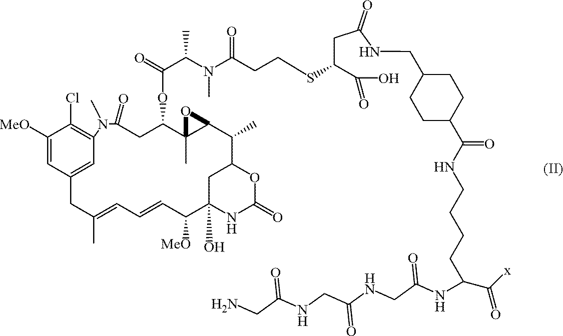

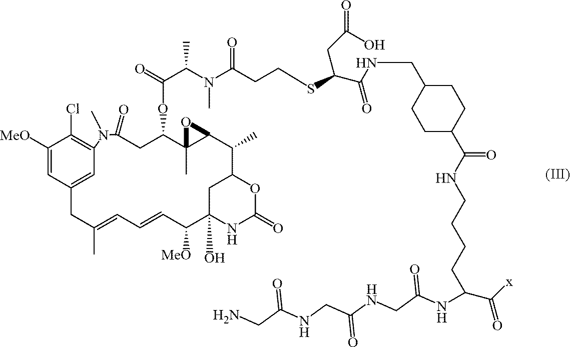

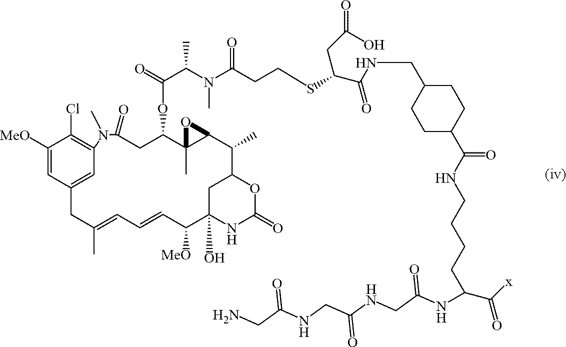

wherein x is —OH or —NH₂. In a specific embodiment, x is—NH₂, and compounds of formulae (i) to (iv) has the structure of formulae (i-1) to (iv-1):

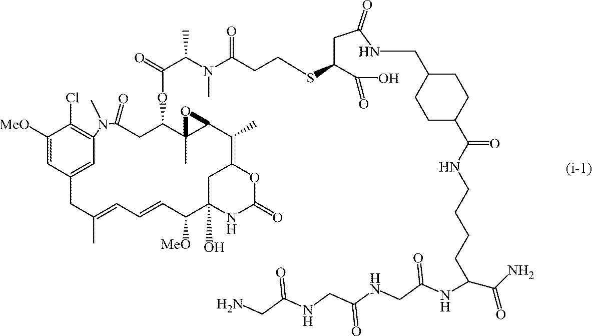 [SMILES: COc1cc2cc(c1Cl)N(C)C(=O)C[C@H](OC(=O)[C@H](C)N(C)C(=O)CCS[C@@H](CC(=O)NCC1CCC(C(=O)NCCCCC(NC(=O)CNC(=O)CNC(=O)CN)C(N)=O)CC1)C(=O)O)[C@]1(C)O[C@H]1[C@H](C)C1C[C@@](O)(NC(=O)O1)[C@H](OC)/C=C/C=C(\C)C2]

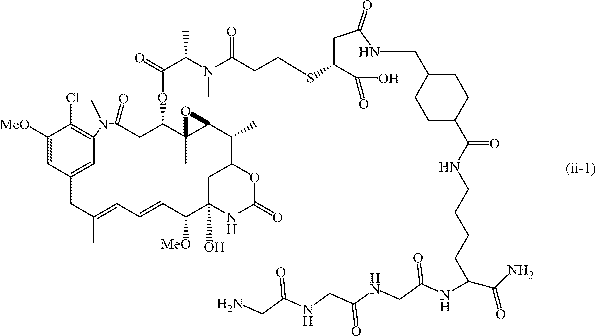 [SMILES: COc1cc2cc(c1Cl)N(C)C(=O)C[C@H](OC(=O)[C@H](C)N(C)C(=O)CCS[C@H](CC(=O)NCC1CCC(C(=O)NCCCCC(NC(=O)CNC(=O)CNC(=O)CN)C(N)=O)CC1)C(=O)O)[C@]1(C)O[C@H]1[C@H](C)C1C[C@@](O)(NC(=O)O1)[C@H](OC)/C=C/C=C(\C)C2]

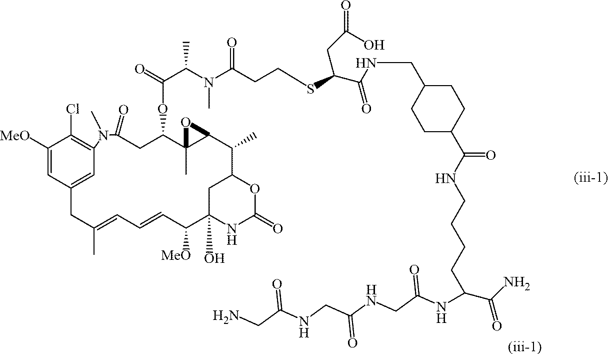

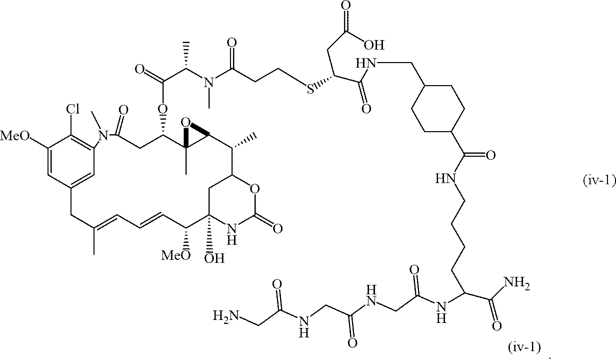

In a particular embodiment, compounds (i) and (ii) are obtained in separate products in step (2); and compounds (iii) and (iv) are obtained in separate products in step (4).

In another particular embodiment, in step (2), compounds (i) and (ii) are obtained in separate products, and compounds (iii) and (iv) are obtained in Mixture 2.

In an embodiment, Mixture 1 is the reaction mixture resulted from the ring-opening reaction of the thiosuccinimide group in the following compound

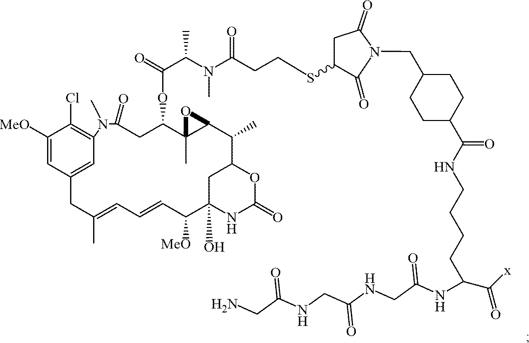

wherein x is —OH or —NH₂.

In a particular embodiment, the separation range in the reverse-phase chromatography of step (2) is as follows:

B in gradient from about 43% to about 53%, with the remainder being A, over 10 to 100 minutes, preferably over 30 to 50 minutes.

In a particular embodiment, the separation range in the reverse-phase chromatography of step (2) is as follows:

B in gradient from about 43% to about 48%, with the remainder being A, over 10 to 100 minutes, preferably over 48 minutes.

In a particular embodiment, the column temperature for the reverse-phase chromatography of step (2) is about 10 to about 40° C., preferably about 20 to about 30° C. In another particular embodiment, loading of the reverse-phase column chromatography is no greater than 20 g/Kg, no greater than 19 g/Kg, no greater than 18 g/Kg, no greater than 17 g/Kg, no greater than 16 g/Kg, no greater than 15 g/Kg, no greater than 14 g/Kg, no greater than 13 g/Kg, no greater than 12 g/Kg, no greater than 11 g/Kg, no greater than 10 g/Kg, no greater than 9 g/Kg, no greater than 8 g/Kg, no greater than 7 g/Kg, no greater than 6 g/Kg. In a preferable embodiment, loading of the reverse-phase column chromatography is no greater than 14 g/Kg. In a very special embodiment, loading of the reverse-phase chromatography is about 0.1 g/Kg to 14 g/Kg, preferably 0.1 g/Kg to 14 g/Kg, and the flow rate of the mobile phase is constant and is in a range of about 100±50 mL/min to about 500±50 mL/min, especially about 300±50 mL/min.

In another particular embodiment, the separation range in the reverse-phase chromatography of step (4) is as follows:

D in gradient from about 22% to about 42%, with the remainder being C, over 20 to 100 minutes.

In another particular embodiment, the separation range in the reverse-phase chromatography of step (4) is as follows:

D in gradient from about 22% to about 28%, with the remainder being C, over 44 minutes.

In a particular embodiment, the column temperature for the reverse-phase chromatography of step (2) is about 10 to about 40° C., preferably about 20 to about 30° C. In another particular embodiment, loading of the reverse-phase column chromatography is no greater than 20 g/Kg, no greater than 19 g/Kg, no greater than 18 g/Kg, no greater than 17 g/Kg, no greater than 16 g/Kg, no greater than 15 g/Kg, no greater than 14 g/Kg, no greater than 13 g/Kg, no greater than 12 g/Kg, no greater than 11 g/Kg, no greater than 10 g/Kg, no greater than 9 g/Kg, no greater than 8 g/Kg, no greater than 7 g/Kg, no greater than 6 g/Kg. In a preferable embodiment, loading of the reverse-phase column chromatography is no greater than 14 g/Kg. In a very special embodiment, loading of the reverse-phase chromatography is about 0.1 g/Kg to 14 g/Kg, preferably 0.1 g/Kg to 14 g/Kg, and the flow rate of the mobile phase is constant and is in a range of about 100±50 mL/min to about 500±50 mL/min, especially about 300±50 mL/min.

### Process for Analysis

The inventors unexpectedly found that the process for separation provided in the first aspect of the disclosure can be used effectively applied in the analysis of the four compounds comprised by Mixture 1 or Mixture 2. For example, the efficacy of the process for separation can be determined under substantially the same chromatographic conditions in an analytical scale with satisfactory precision.

Therefore, in a second aspect, provided is a process for analyzing one or more compounds in Mixture 1 which comprises four compounds, each of the four compounds in the mixture comprising Moiety 1, Moiety 2 and Moiety 3.

the process comprising applying the step (2) in an analytical scale; wherein Moiety 1, Moiety 2, Moiety 3 and the step (2) are as defined above.

In one embodiment, the process further comprises applying the step (4) in an analytical scale; wherein the step (4) is as defined above.

In a third aspect, provided is a process for analyzing one or more compounds in Mixture 2 which comprises two compounds, each of the two compounds in the mixture comprising Moiety 1, Moiety 2 and Moiety 3.

the process comprising applying the step (4) in an analytical scale; wherein Moiety 1, Moiety 2, Moiety 3 and the step (4) are as defined above.

In one embodiment, the chromatographic conditions of the process for analysis are optionally studied for robustness. In one embodiment, chromatographic conditions of the process for analysis are adjusted according to the result of system suitability assays. Each process for analysis may serve independently as the in-process control method for one or more preparation processes. In one embodiment, the chromatographic conditions are the same for the preparation process and the process for analysis. In another embodiment, only the particle size and/or the pore size of the stationary phase are different between the chromatographic conditions used in the preparation process and those used in the process for analysis. In one embodiment, both the preparation process and the process for analysis use gradient elution. In another embodiment, the preparation process uses gradient elution and the process for analysis uses isocratic elution.

In a particular embodiment, the loading volume of the process for analysis is less than 1 ml. In another particular embodiment, the loading volume of the process for analysis is 1-300 µL, for example 5-100 µL. In a very special embodiment, the flow rate of the mobile phase is no greater than 1 mL/min, for example, the flow rate may be 0.5-1 mL/min, especially 0.7 mL/min, 0.8 mL/min, 0.9 mL/min or 1.0 mL/min.

### The Isomeric Compounds According to the Disclosure

### Payload-Linker Intermediate

In a fourth aspect, provided is a compound with the structure of any one of the following formulae (XI) to (XIV):

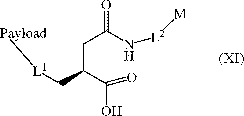

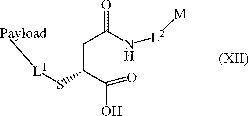

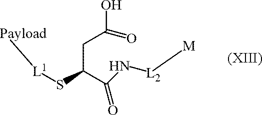

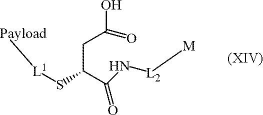

wherein

Payload, L¹, L² and M are as defined above.

In one embodiment, L¹ is not present, L² is

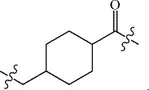

M is

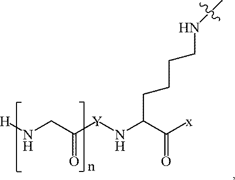

and the formulae (XI) to (XIV) has the following structures of formulae (XI-1) to (XIV-1), respectively.

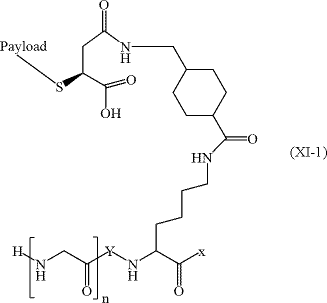

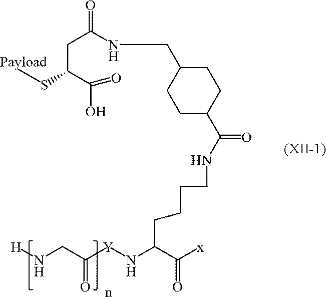

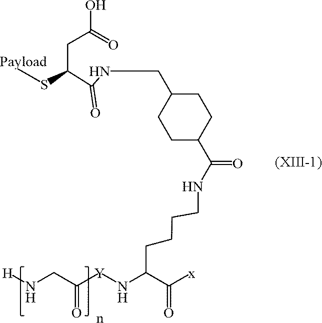

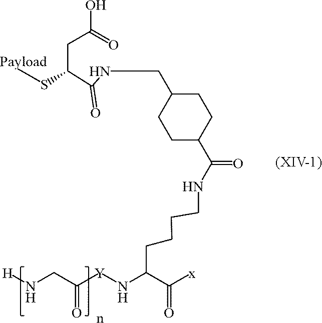

wherein

Payload, Y, n and x are as defined above.

Formulae (XI) and (XII) are referred to as β, and the configuration of formulae (XIII) and (XIV) are referred to as α, according to the position of the thiol group.

In one embodiment, Y is a bond and n is 3, and the formulae (XI-1) to (XIV-1) has the following structures of formulae (XI-1-1) to (XIV-1-1), respectively.

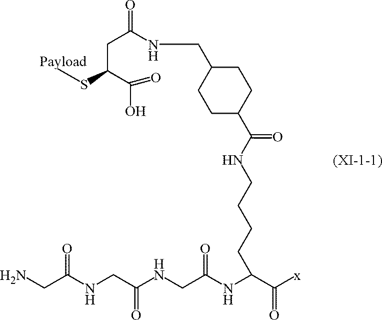

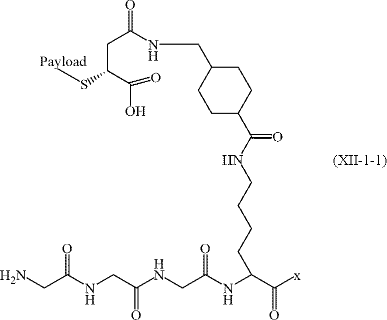

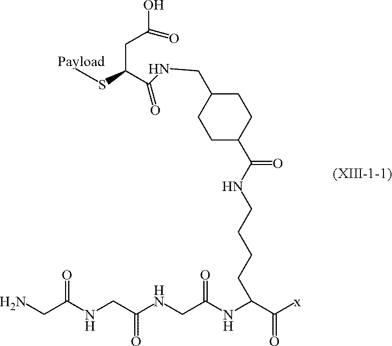

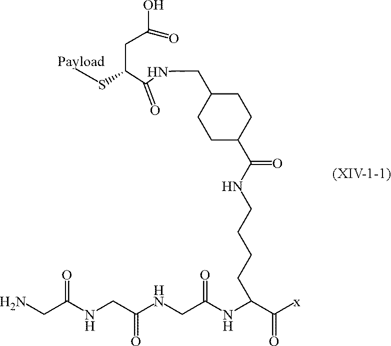

In a particular embodiment, Payload is a cytotoxin moiety containing one or more chiral centers, preferably a maytansinoid, more preferably DM1. In one embodiment, Payload is DM1, and the of formulae (XI-1-1) to (XIV-1-1) represent the structures of the isomeric compounds (i) to (iv), which are as defined above.

In a fifth aspect, provided is a mixture comprising two compounds, wherein each compound has the structure of any one of the formulae (XI) to (XIV), provided that the two compounds are of different structures. In one embodiment, the mixture comprises two compounds, having the structure of formula (XI) and formula (XII), respectively. In another embodiment, the mixture comprises two compounds, having the structure of formula (XIII) and formula (XIV), respectively. In one embodiment, the mixture comprises two compounds, having the structure of formula (XI-1) and formula (XII-1), respectively. In another embodiment, the mixture comprises two compounds, having the structure of formula (XIII-1) and formula (XIV-1), respectively. In one embodiment, the mixture comprises two compounds, having the structure of formula (XI-1-1) and formula (XII-1-1), respectively. In another embodiment, the mixture comprises two compounds, having the structure of formula (XIII-1-1) and formula (XIV-1-1), respectively. In a particular embodiment, the mixture comprises two compounds, having the structure of formula (i) and formula (ii), respectively. In another embodiment, the mixture comprises two compounds, having the structure of formula (iii) and formula (iv), respectively.

The mixture of compounds (iii) and (iv) are referred to as α intermediates or α isomers hereinbelow.

In some embodiments, the compounds provided by the present disclosure can be describe as below:

<1> A compound of formula (XI), (XII), (XIII) or (XIV):

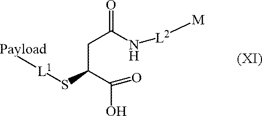

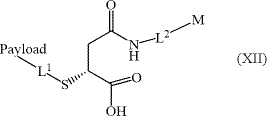

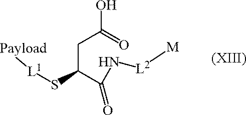

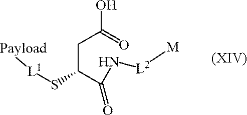

wherein

Payload is a cytotoxin moiety containing one or more chiral centers;
L¹ is not present, or is a bivalent group being one or more selected from the group consisting of C₁₋₁₀ alkylene, C₃₋₁₀ cycloalkylene, C₆-₁₀ arylene, 4 to 10 membered heterocyclylene, 5 to 10 membered heteroarylene, —NH—, —(CO)—, —NH(CO)— and —(CO)NH—;
L² is a bond, or is a bivalent group being one or more selected from the group consisting of C₁₋₁₀ alkylene, C₃₋₁₀ cycloalkylene, C₆-₁₀ arylene, 4 to 10 membered heterocyclylene, 5 to 10 membered heteroarylene, —NH—, —(CO)—, —NH(CO)— and —(CO)NH—; and
M comprises a ligase recognition motif,
wherein the compound is in predominantly, e.g., in pure, or substantially pure, isomeric form, e.g., substantially free of other isomeric forms, e.g., having a purity of greater than 90%, e.g., greater than 95%, e.g., greater than 98%, e.g., a least 99%.

<2> The compound of <1> which is a compound of formula (XIII) or (XIV).

<3> The compound of <1> or <2>, wherein the ligase recognition motif is selected from (a) LPXTG, wherein X is any amino acid residue, e.g., LPETG, and (b) Gₙ, wherein G is glycine (Gly), and n is an integer of 3 to 10.

<4> The compound of <3> where M is H-Gly-Gly-Gly-Lys-NH₂, and the M is linked to L² via the ε-amino on the lysine.

<5> The compound of any one of <1> to <4> wherein L¹ is not present, and L² is selected from

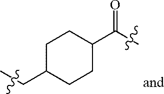

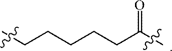

<6> The compound of any one of <1> to <4> where L¹ is not present, L² is

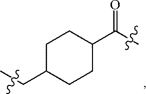

M is

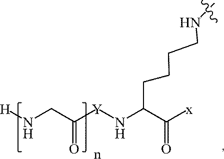

wherein
Y is a bond, or is a spacer sequence of 1-20 amino acids;
n is an integer of 3 to 10, e.g., 3; and
x is selected from the group consisting of hydrogen, —OH, —NH₂, an amino acid fragment comprising 1-10 amino acids, and a nucleotide fragment comprising 1-10 nucleotides, e.g., selected from the group consisting of —OH, —NH₂, and an amino acid fragment comprising 1-10 amino acids; e.g., selected from the group consisting of —OH, —NH₂ and Gly, e.g., —NH₂.

<7> The compound of any one of <1> to <6> wherein the cytotoxin comprises a thiol moiety capable of reaction with a maleimide moiety.

<8> The compound of any one of <1> to <7> wherein the cytotoxin is a maytansinoid, e.g., DM1:

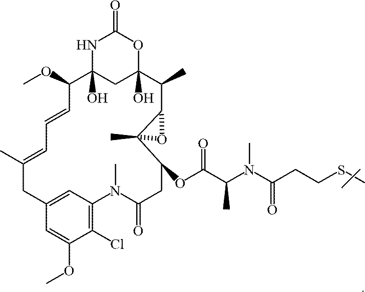

<9> The compound of any one of <1> to <8>, selected from compounds of formula (i), (ii), (iii) or (iv)

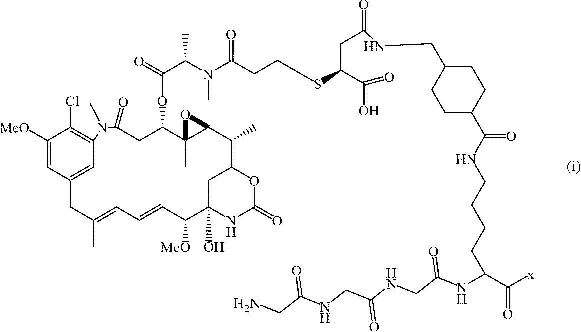

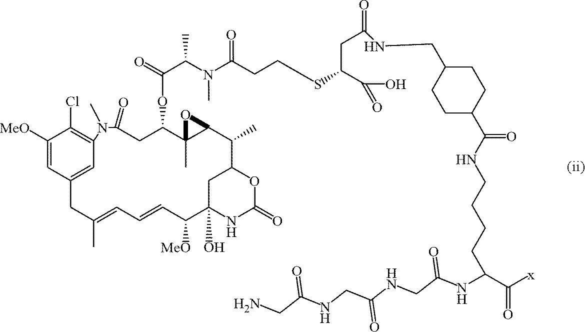

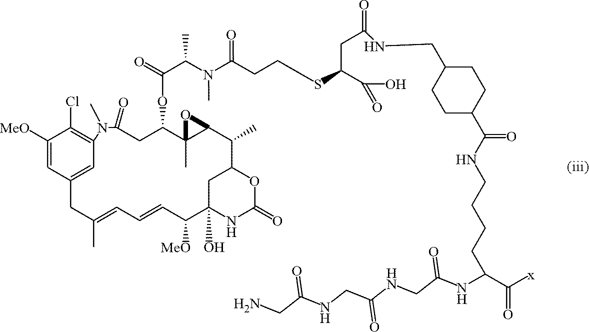

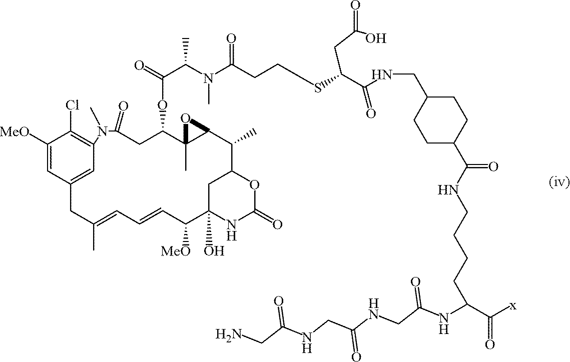

wherein x is —OH or —NH₂.

<10> The compound of <9> which is a compound of formula (iii) or (iv).

In an aspect, the present disclosure provides a process of making a compound of formula (XI), (XII), (XIII) or (XIV), which is as follows:

<17> A process of making a compound of formula (XI), (XII), (XIII) or (XIV), comprising
(i) synthesizing a mixture of compounds of formulae (XI), (XII), (XIII) and (XIV);
(ii) subjecting the mixture to chromatography so as to obtain the target compound(s),
   wherein each of the target compound(s) are obtained in a separate product, or two target compounds are obtained in a second mixture; and
(iii) optionally recovering an eluate collected in step (ii) comprising a third mixture, wherein the third mixture comprises one or more additional target compounds which are different from those separated in step (2), and subjecting the eluate recovered to chromatography to separate the additional target compound(s).

<18> The process of <17> wherein the chromatography is reverse-phase chromatography.

### Antibody Drug Conjugate

The compound of formula (XI), (XII), (XIII) or (XIV) can be coupled with a biomolecule containing a ligase recognition motif, forming a bioconjugate, wherein the ligase recognition motif comprised by the biomolecule is the counterpart of the ligase recognition motif comprised by Amino acid sequence 1 in the structure M of the compound of formula (XI), (XII), (XIII) or (XIV).

In one embodiment, the compound of formula (XI), (XII), (XIII) or (XIV) comprises a ligase acceptor substrate recognition motif GGG, and the biomolecule comprises a ligase donor substrate recognition motif LPXTG.

In a sixth aspect, provided is an antibody drug conjugate (ADC) prepared using the compound of formula (XI), (XII), (XIII) or (XIV) and an antibody or the antigen binding fragment thereof. The preparation of ADC can be performed using any method known in the art, for example by coupling the ligase recognition motif comprised by the structure M with a ligase recognition motif comprised by the antibody or the antigen binding fragment thereof. The resulted ADC also contains the isomeric ring-opened thiosuccinimide group as in the compound of formula (XI), (XII), (XIII) or (XIV), and the resulted ADC has the structure of any one of the following formulae (XVII-1) to (XX-1):

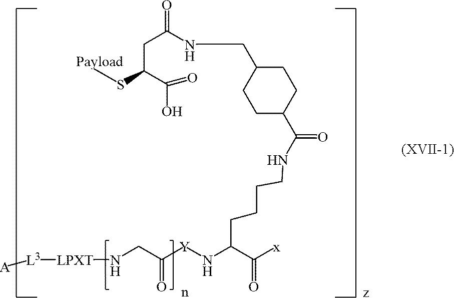

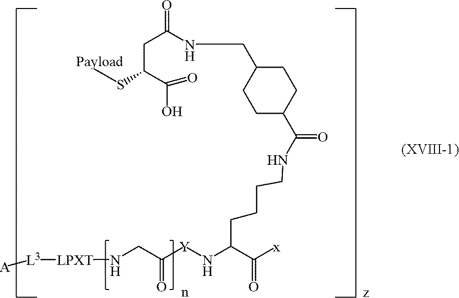

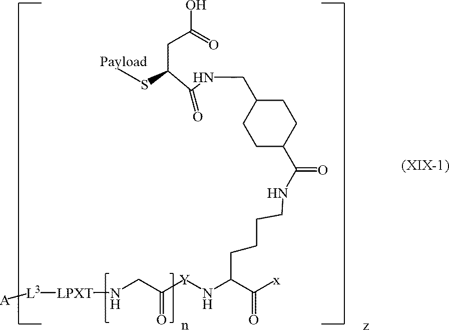

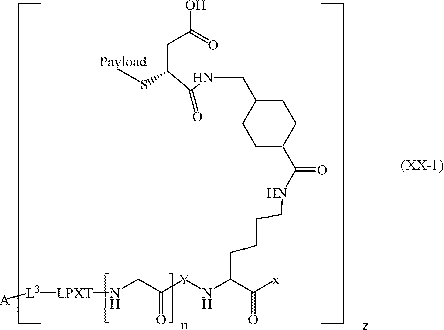

wherein
A is an antibody or an antigen binding fragment thereof, which is optionally modified to have the recognition motif of the ligase donor substrate or the recognition motif of the ligase acceptor substrate;
L³ is absent, or comprises: (1) Amino acid sequence 4 comprising 1-20 amino acids, (2) an optional PEG fragment, which comprises a “—(C₂H₄—O)ⱼ—” structure, j being an integer of 1 to 100, and (3) an optional group U, which is bivalent and is selected from the group consisting of Cleavable sequence 2, spacer Sp2 comprising 2-100 amino acids, and the combination thereof;
Cleavable sequence 2 comprises Amino acid sequence 5 which can be cleaved by enzyme, and Cleavable sequence 2 comprises 1-10 amino acids;
Payload, Y, n and x are as defined above.

### In One Embodiment, J Is an Integer of 1 to 10

In one embodiment, Sp2 is a spacer sequence containing 2-20 amino acids. In a particular embodiment, Sp2 is a spacer sequence selected from the group consisting of GA, GGGS and GGGGSGGGGS, especially GA.

In one embodiment, the introduction position of the recognition motif of the ligase substrate is not limited, for example, its introduction position can be, but not limited to, located at the C-terminal or the N-terminal of the heavy chain or light chain of the antibody.

In a preferred embodiment, the light chain of the antibody or antigen-binding fragment thereof includes 3 types: wild-type (LC); the C-terminus modified light chain (LCCT), which is modified by direct introduction of an ligase recognition motif LPXTG, and C-terminus modified light chain (LCCT_{L}), which is modified by introduction of short peptide spacers plus the ligase donor substrate recognition motif LPXTG. The heavy chain of the antibody or antigen-binding fragment thereof includes 3 types: wild-type (HC); the C-terminus modified heavy chain (HCCT), which is modified by direct introduction of an ligase recognition motif LPXTG; and C-terminus modified heavy chain (HCCT_{L}), which is modified by introduction of short peptide spacers plus the ligase donor substrate recognition motif LPXTG. X can be any natural or non-natural single amino acid. When z in the compound of formula (IV) is 1 or 2, the combination of the above heavy and light chains can form 8 preferred antibody molecules. The sortase mediated ligation results in the formation of a new amide bond between the C-terminal sorting motif LPXTG and an N-terminal GGG, wherein the conjugation reaction proceeds by first cleaving the peptide bond between the threonine and glycine residues, then ligating the LPXT to the GGG.

In a preferred embodiment, the light chain of the antibody or antigen-binding fragment thereof includes 3 types: wild-type (LC); the N-terminus modified light chain (LCNT), which is modified by direct introduction of an ligase recognition motif GGG; and N-terminus modified light chain (LCNT_{L}), which is modified by introduction of short peptide spacers plus the ligase acceptor substrate recognition motif GGG. The heavy chain of the antibody or antigen-binding fragment thereof includes 3 types: wild-type (HC); the N-terminus modified heavy chain (HCNT), which is modified by direct introduction of an ligase recognition motif GGG; and N-terminus modified heavy chain (HCNT_{L}), which is modified by introduction of short peptide spacers plus the ligase acceptor substrate recognition motif GGG.

In one embodiment, Y is a bond and n is 3, and the formulae (XVII-1) to (XX-1) has the following structures of formulae (XVII-1-1) to (XX-1-1), respectively.

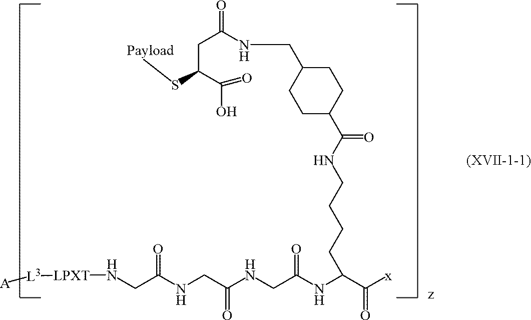

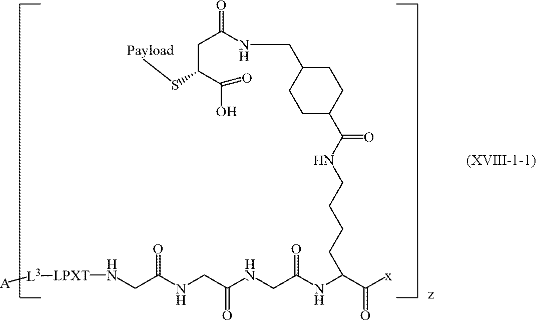

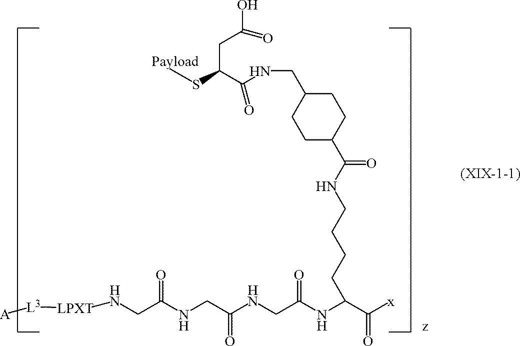

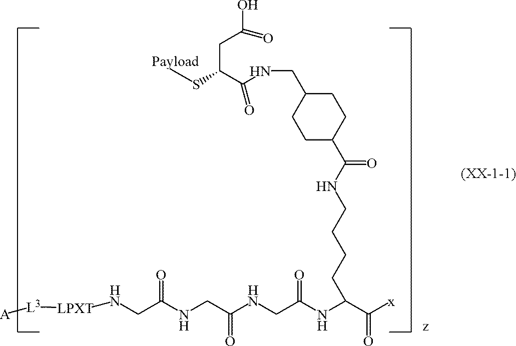

wherein

Payload, A, x and z are as defined above.

In one embodiment, Payload is DM1, and z is 2, and the formulae (XI-1) to (XIV-1) has the following structures of formulae (XI-1-1) to (XIV-1-1), respectively.

In a particular embodiment, Payload is a cytotoxin moiety containing one or more chiral centers. The cytotoxin is preferably a maytansinoid, more preferably DM1. In one embodiment, Payload is DM1, L³ is absent, and the of formulae (XVII-1-1) to (XX-1-1) have the structures of the following compounds (v) to (viii), respectively.

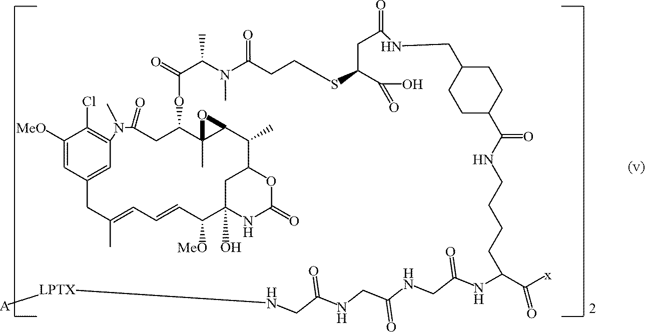

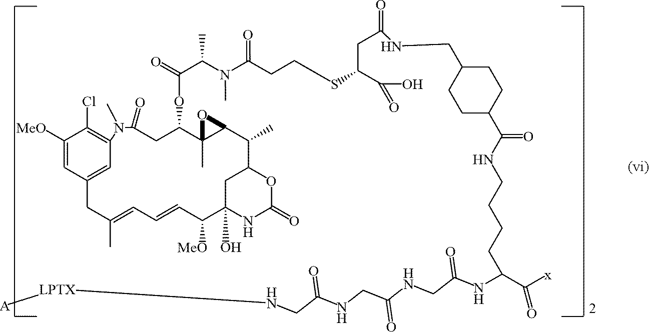

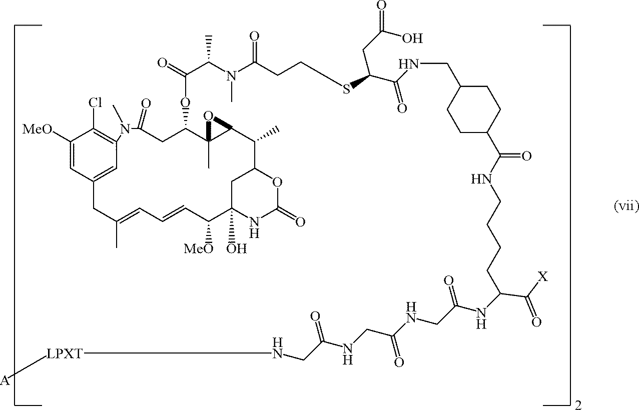

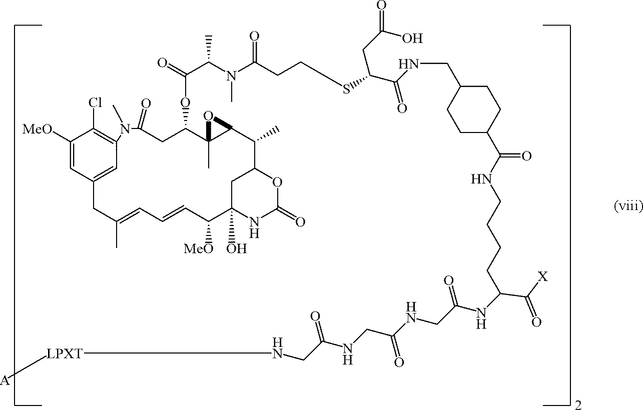

wherein

A and x are as defined above. In a specific embodiment, x is —NH₂.

In one embodiment, A is Trastuzumab, which is optionally modified to have one of the recognition motif of the ligase donor substrate and the recognition motif of the ligase acceptor substrate. In a preferable embodiment, A is Trastuzumab with a C-terminus modified light chain (LCCT_{L}), which is modified by introduction of short peptide spacers plus the ligase donor substrate recognition motif LPXTGJ, wherein J is as defined above. In one embodiment, the short peptide spacer is GA. In another embodiment, the heavy chain of the antibody or antigen-binding fragment thereof is a wild-type heavy chain (HC).

In a seventh aspect, provided is a mixture comprising two compounds, wherein each compound has the structure of any one of the formulae (XVII-1) to (XX-1), provided that the two compounds are of different structures. In one embodiment, the mixture comprises two compounds, having the structure of formula (XVII-1) and formula (XVIII-1), respectively. In another embodiment, the mixture comprises two compounds, having the structure of formula (XIX-1) and formula (XX-1), respectively. In one embodiment, the mixture comprises two compounds, having the structure of formula (XVII-1-1) and formula (XVIII-1-1), respectively. In another embodiment, the mixture comprises two compounds, having the structure of formula (XIX-1-1) and formula (XX-1-1), respectively. In a particular embodiment, the mixture comprises two compounds, having the structure of formula (v) and formula (vi), respectively. In another embodiment, the mixture comprises two compounds, having the structure of formula (vii) and formula (viii), respectively. The mixture of compounds (vii) and (viii) are referred to as o ADCs hereinbelow.

In some embodiments, the antibody drug conjugates provided by the present disclosure can be describe as below:

<11> An antibody drug conjugate comprising a ring-opened thiosuccinimide structure of formula (I), (II), (III), or (IV):

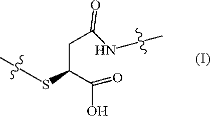

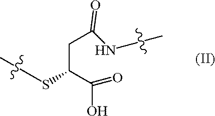

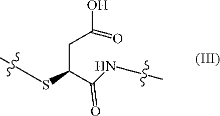

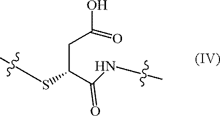

wherein the compound is in predominantly, e.g., in pure, or substantially pure, isomeric form, e.g., substantially free of other isomeric forms, e.g., having a purity of greater than 90%, e.g., greater than 95%, e.g., greater than 98%, e.g., a least 99%.

<12> The antibody drug conjugate of <11> formed by reacting a compound according to any one of <1> to <11> with an antibody in the presence of a ligase.

<13> The antibody drug conjugate of <11> or <12> having the formulae (XVII-1-1) to (XX-1-1), respectively

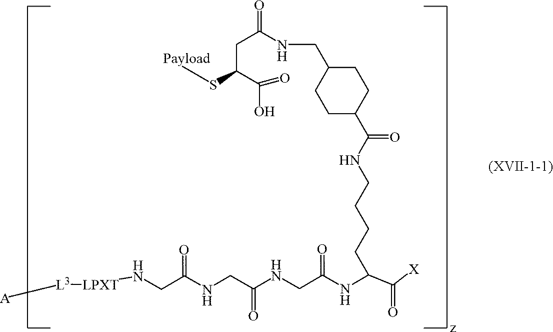

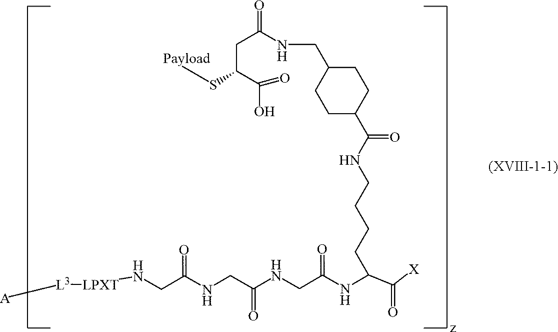

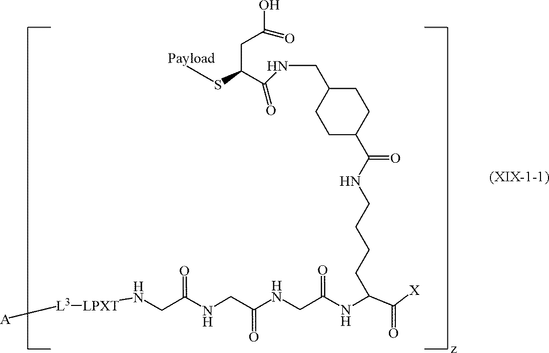

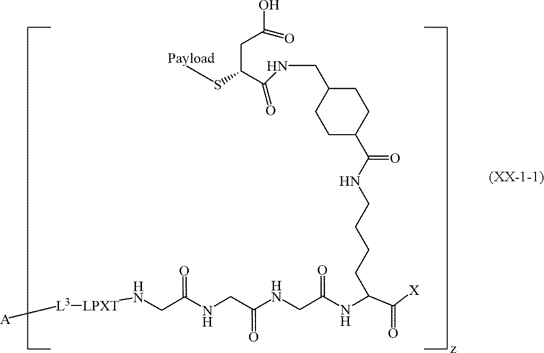

wherein
Payload and x are as defined above, e.g., is a cytotoxin moiety containing one or more chiral centers, e.g., the rest moiety of a maytansinoid molecule except for the thiol moiety, e.g., the rest moiety of DM1 except for the thiol moiety;
A is an antibody or an antigen binding fragment thereof, which is optionally modified to have the recognition motif of a ligase donor substrate or the recognition motif of a ligase acceptor substrate;
for example, A is an antibody which binds human epidermal growth factor receptor 2 (HER2), e.g., trastuzumab, which is modified to have the recognition motif of the ligase donor substrate or the recognition motif of the ligase acceptor substrate.
z is an integer 1 or 2.

<14> The antibody drug conjugate of <13> wherein A is Trastuzumab with a C-terminus modified light chain (LCCT_{L}), which is modified by introduction of a short peptide spacer, e.g., -GA-, plus the ligase donor substrate recognition motif LPXTGJ, wherein J is absent, or is an amino acid fragment comprising 1-10 amino acids.

<15> The antibody drug conjugate of <13> or <14> wherein Payload is DM1, L³ is absent, and formulae (XVII-1-1) to (XX-1-1) have the structures (v) to (viii), respectively

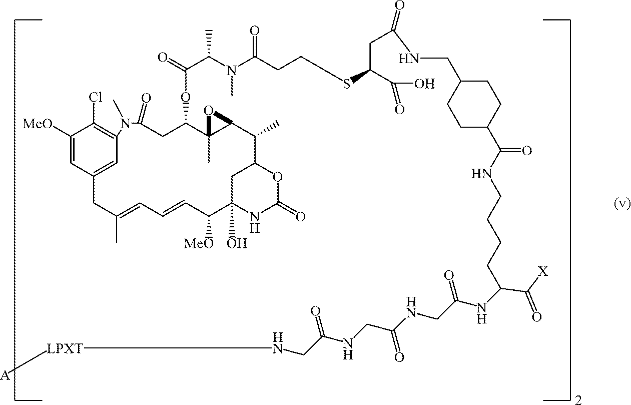

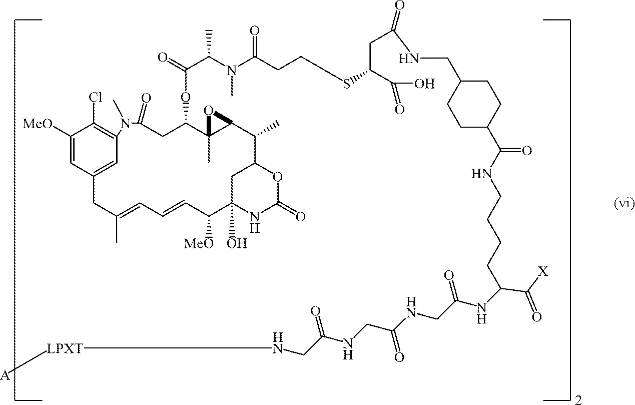

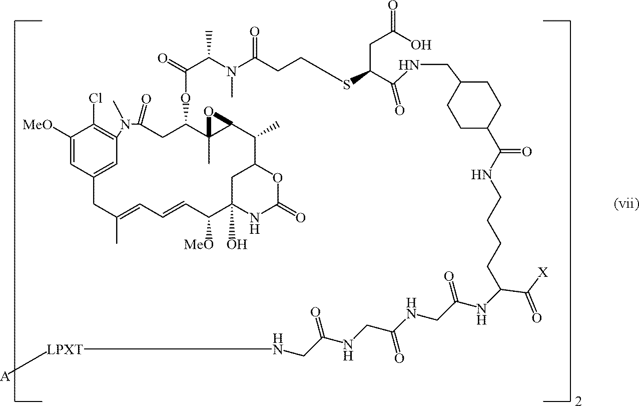

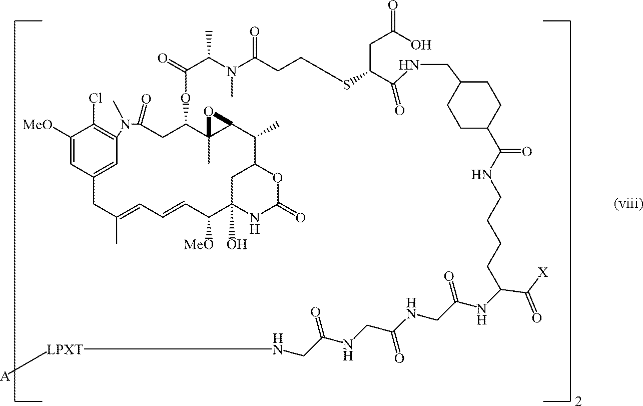

wherein x is —OH or —NH₂.

<16> The antibody drug conjugate of <14> having structure (vii) as set forth in <15>.

### Compounds Mixture and Antibody Drug Conjugate Mixture

In some embodiments, the α isomers of the present disclosure is provided in the form of mixture, and therefore, in the eighth aspect, the present disclosure provides a composition comprising α isomers of the present disclosure. In some embodiments, the composition of the present disclosure can be describe as below:

<45> A composition comprising a mixture, e.g., a racemic mixture, of compounds of formula (XIII) and (XIV):

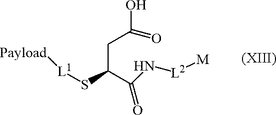

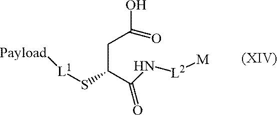

wherein

Payload is a cytotoxin moiety containing one or more chiral centers;
L¹ is not present, or is a bivalent group being one or more selected from the group consisting of C₁₋₁₀ alkylene, C₃₋₁₀ cycloalkylene, C₆-₁₀ arylene, 4 to 10 membered heterocyclylene, 5 to 10 membered heteroarylene, —NH—, —(CO)—, —NH(CO)— and —(CO)NH—;
L² is a bond, or is a bivalent group being one or more selected from the group consisting of C₁₋₁₀ alkylene, C₃₋₁₀ cycloalkylene, C₆-₁₀ arylene, 4 to 10 membered heterocyclylene, 5 to 10 membered heteroarylene, —NH—, —(CO)—, —NH(CO)— and —(CO)NH—; and
M comprises a ligase recognition motif,
wherein the composition is substantially free (e.g., at least 90%, at least 95%, at least 98% or at least 99% free) of compounds of Formula XI and XII

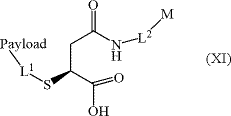

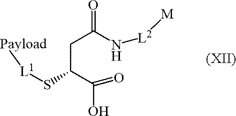

<46> The composition of claim <45>, wherein the ligase recognition motif is selected from (a) LPXTG, wherein X is any amino acid residue, e.g., LPETG, and (b) Gₙ, wherein G is glycine (Gly), and n is an integer of 3 to 10.

<47> The composition of claim <45> or <46> where M is H-Gly-Gly-Gly-Lys-NH₂, and the M is linked to L² via the ε-amino on the lysine.

<48> The composition of any of claims <45>-<47> wherein L¹ is not present, and L² is selected from

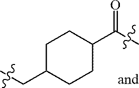

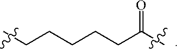

<49> The composition of any of claims <45>-<48>, where L¹ is not present, L² is

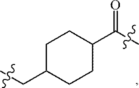

M is

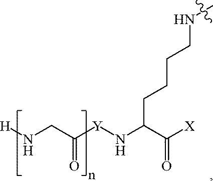

wherein
Y is a bond or is a spacer sequence of 1-20 amino acids;
n is an integer of 3 to 10, e.g., 3; and
x is selected from the group consisting of hydrogen, —OH, —NH₂, an amino acid fragment comprising 1-10 amino acids, and a nucleotide fragment comprising 1-10 nucleotides, e.g., selected from the group consisting of —OH, —NH₂, and an amino acid fragment comprising 1-10 amino acids; e.g., selected from the group consisting of —OH, —NH₂ and Gly, e.g., —NH₂.

<50> The composition of any of claims <45>-<49> wherein the cytotoxin comprises a thiol moiety capable of reaction with a maleimide moiety.

<51> The composition of any of <45>-<50> wherein the cytotoxin is a maytansinoid, e.g., DM 1:

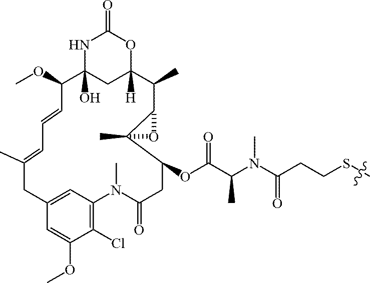

<52> The composition of any of <45>-<47> which is a mixture of compounds of formulae (iii) and (iv) and substantially free of compounds of formula (i) and (ii)

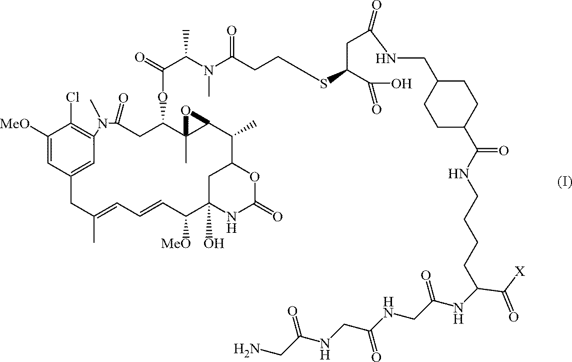

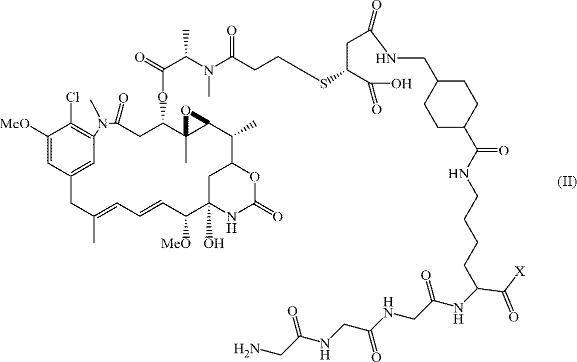

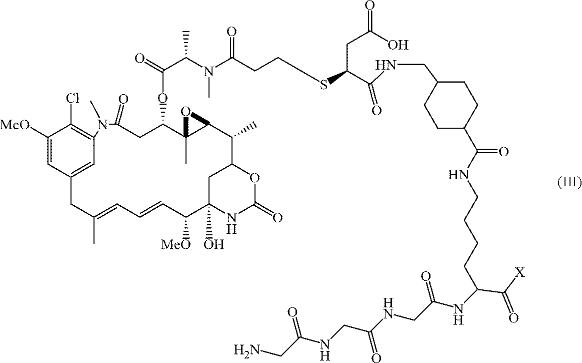

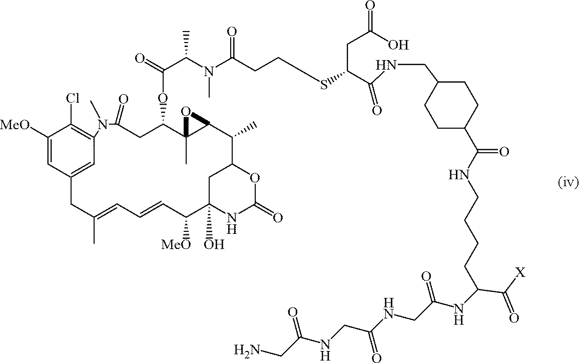

In the nineth aspect, the present disclosure provides an antibody drug conjugate composition comprising a mixture of the α-ADCs of the present disclosure. In some embodiments, the antibody drug conjugate composition of the present disclosure can be describe as below:

<53> An antibody drug conjugate composition comprising a mixture, e.g., a racemic mixture, of antibody drug conjugate comprising a ring-opened thiosuccinimide structure of formula (III) and antibody drug conjugate comprising a ring-opened thiosuccinimide structure of formula (IV):

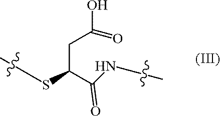

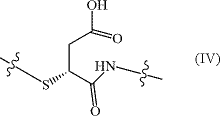

wherein the composition is substantially free (e.g., at least 90%, at least 95%, at least 98% or at least 99% free) of compounds of Formula (I) and (II):

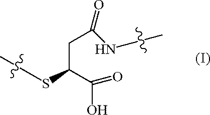

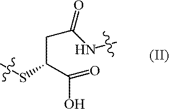

<54> The antibody drug conjugate composition of claim <53> formed by reacting a composition according to any one of claims <45>-<52> with an antibody in the presence of a ligase.

<55> The antibody drug conjugate composition of claims <52>, <53>, or <54> which is a mixture of compounds having the formulae (XIX-1-1) and (XX-1-1):

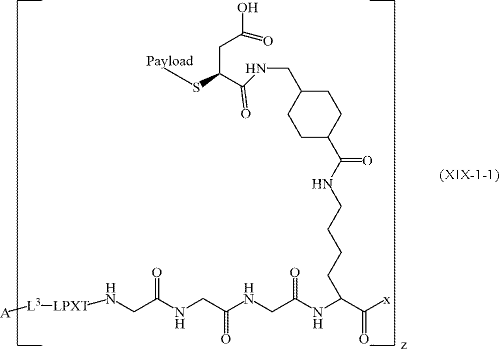

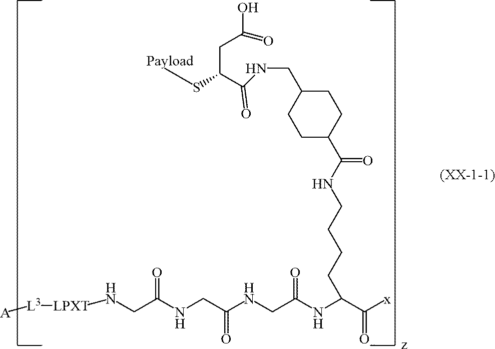

wherein
Payload is as defined above, e.g., is a cytotoxin moiety containing one or more chiral centers, e.g., the rest moiety of a maytansinoid molecule except for the thiol moiety, e.g., the rest moiety of DM1 except for the thiol moiety;
A is an antibody or an antigen binding fragment thereof, which is optionally modified to have the recognition motif of a ligase donor substrate or the recognition motif of a ligase acceptor substrate;
for example, A is an antibody which binds human epidermal growth factor receptor 2 (HER2), e.g., trastuzumab, which is modified to have the recognition motif of the ligase donor substrate or the recognition motif of the ligase acceptor substrate;
z is an integer 1 or 2.

<56> The antibody drug conjugate composition of claim <55> wherein A is Trastuzumab with a C-terminus modified light chain (LCCT_{L}), which is modified by introduction of a short peptide spacer, e.g., -GA-, plus the ligase donor substrate recognition motif LPXTGJ, wherein J is absent, or is an amino acid fragment comprising 1-10 amino acids.

<57> The antibody drug conjugate composition of claim <55> or <56> wherein Payload is DM1, L³ is absent, and formulae (XIX-1-1) and (XX-1-1) have the structures (vii) and (viii), respectively

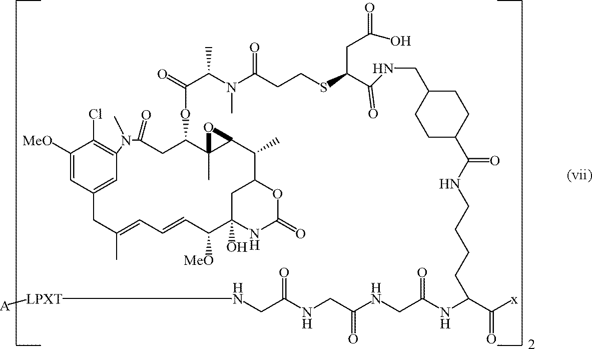

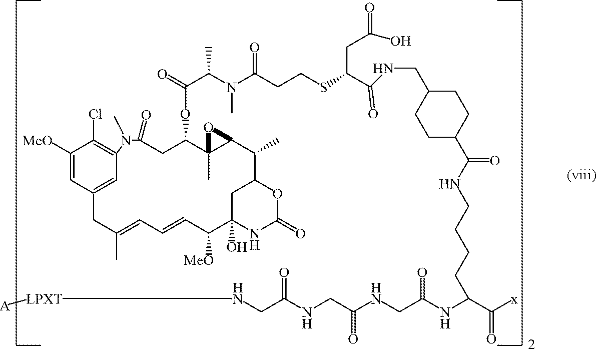

In the tenth aspect, the present disclosure provides treatment method which can be describe as below:

<58> A method of treating cancer in a patient in need thereof, comprising administering to the patient an effective amount of the antibody drug conjugate composition of any one of claims <53>-<57>.

<59> The method of claim <58>, wherein the cancer is HER2-positive cancer, e.g., HER2-positive breast cancer.

<60> The method of claim <58> or <59> wherein the antibody drug conjugate composition is according to claim <57>.

### Pharmaceutical Composition, Formulation and Kit

In another aspect, further provided is a pharmaceutical composition which comprises (a) the compound of formula (XI), (XII), (XIII) or (XIV); or the antibody drug conjugate of the present invention; or the composition comprising a mixture of compounds of formula (XIII) and (XIV); or the antibody drug conjugate composition of the present disclosure; and (b) a pharmaceutically acceptable carrier.

### Treatment and Use

In another aspect, further provided is use of the compound of formula (XI), (XII), (XIII) or (XIV); or the antibody drug conjugate of the present invention; or the composition comprising a mixture of compounds of formula (XIII) and (XIV); or the antibody drug conjugate composition of the present disclosure; or the pharmaceutical composition of the present disclosure in the preparation of a medicament for treating a cancer.

In another aspect, provided is a method of treating a cancer, the method comprises administrating an effective amount of the compound of formula (XI), (XII), (XIII) or (XIV); or the antibody drug conjugate of the present invention; or the composition comprising a mixture of compounds of formula (XIII) and (XIV); or the antibody drug conjugate composition of the present disclosure; or the pharmaceutical composition of the present disclosure to a subject in need thereof.

In yet another aspect, provided is the compound of formula (XI), (XII), (XIII) or (XIV); or the antibody drug conjugate of the present invention; or the composition comprising a mixture of compounds of formula (XIII) and (XIV); or the antibody drug conjugate composition of the present disclosure; or the pharmaceutical composition of the present disclosure, for use in treatment of a cancer.

In one embodiment, the cancer is HER-2 positive.

### Beneficial Effect

The process of the present disclosure achieves at least one of the following technical effects:
(1) The complete separation and purification of four isomer compounds comprised by Mixture can be achieved and the pure isomer compounds are obtained with high efficiency and yield;
   Noteworthily, in the separation process (such as the process in Example 1) and analytical process (such as the process in Example 2) for β1, β2 and α isomers, when the acidic agent used in the mobile phase is TFA, NaH₂PO₄, CH₂O₂, K₂HPO₄, AA, TEAP and NaClO₄ or other conventional reagents, the resolution among the β1, β2 and α isomers are hardly observed. On the contrary, when the acidic agent is selected from CH₃COOH and L-TA, the β1, β2 and α isomers can be successfully separated and purified. Therefore, the inventor unexpectedly find that a specific acidic agent, namely CH₃COOH or a similar alkyl carboxylic acid can efficiently enhance the resolution among the β1, β2 and α isomers, and thus can be applied in separation and/or purification process.
(2) The process is easy to conduct, cost-effective, and has been proved to be efficient in pilot scale manufacture, and thus is advantageous for industrial production.
(3) The efficacy of separation for complicated samples is typically monitored by complicated and costly after-column detections, such as LCMS methods using expensive chromatographic columns for precise analysis and in some cases requiring preprocessing of the samples to eliminate the remaining non-volatile salt from the products gained by separation.

The analysis process of the present disclosure adopts chromatographic conditions similar to those used in the separation process, and therefore less adjustments are need when designing the chromatographic method for separation process or the chromatographic method for analysis process, facilitating easier chromatographic processing for complicated samples. In addition, volatile agents are used in process for separation of the present disclosure. Therefore, the products gained by separation do not contain non-volatile salt and therefore can be directly subject to LCMS analysis without preprocess for desalting. This is especially advantageous when Mixture 1 is the reaction mixture of a ring-opening reaction. When there is a need, impurities or byproducts could be detected promptly.
(4) The process demonstrates high efficacy, for example high resolution (parameter R in chromatography) and satisfying precision, etc.
(5) The isolated isomers tend to show differences in the cytotoxicity. Isomer α2 demonstrate 2 fold stronger activity than β isomers (the mixture of β1 isomer and β2 isomer) in the human breast cancer HCC1954 cell proliferation assay.

The α-ADCs of the present disclosure achieves at least one of the following technical effects:

α-ADCs have significant antitumor effect, and can be enriched to tumor and maintain in situ stability. The lower dose of the α-ADCs caused similar efficacy compared to Kadcyla, demonstrating superior efficacy of the α-ADCs of the present disclosure.

### EXAMPLES

In order to illustrate the objects and technical solutions more clearly, the present disclosure is further described below with reference to specific examples. It is to be understood that the examples are not intended to limit the scope of the disclosure. The specific experimental methods which are not mentioned in the following examples are carried out according to conventional experimental methods.

Abbreviations

| | | | |
|---|---|---|---|
| AA | ammonium acetate | L-TA | L-tartaric acid |
| TFA | trifluoroacetic acid | TEAP | triethylamine phosphate |
| DAR | drug to antibody ratio | MRT | mean residence time |
| LLOQ | lowest limit of quantification | ADA | anti-drug antibody |
| RTV | relative tumor volume | TGI | tumor growth inhibition |
| ALT | alanine aminotransferase | AST | aspartate aminotransferase |
| PLT | platelet count | TAb | total antibody |
| GLOB | globulin | A/G | albumin/globulin |
| TK | toxicokinetic | RLU | relative light unit |
| APTT | activated partial thromboplastin time | | |
| PAB | p-aminobenzyloxycarbonyl, also known as PABC | | |

### Instruments, Materials and Reagents

Unless otherwise stated, the instruments and reagents are commercially available or can be prepared according to conventional means in the art. The reagents can be used directly without further purification.

¹HNMR, ¹³C NMR, ¹H-¹H COSY and HMBC spectra were recorded on Bruker AV-600, 600 MHz. Chemical shifts are expressed in parts per million (ppm). Coupling constants are in units of hertz (Hz). Splitting patterns describe apparent multiplicities and are designated as s (singlet), d (doublet), t (triplet), q (quartet), m (multiplet) and br (broad).

Cell viability is tested using CellTiter-Glo® Luminescent Kit (Promega, Cat.# G7573) read by a Biotek Cytation3 Imaging reader.

HCC1954 human breast cancer xenograft model (HCC1954 model) and NCI-N87 human gastric cancer xenograft model (NCI-N87 model) in female BALB/c nude mice are provided by ATCC. Cynomolgus monkeys are provided by GuangZhou XiangGuan, Ltd..

PLT, APTT, AST, ALT, GLOB and A/G are tested on Hematology analyzer (ADVIA 2120i Siemens) and auxiliary reagent.

Kadcyla is provided by Roche.

### Preparation of the Mixture Containing Β Isomers and Α Isomers

A compound with the following structure was prepared and the ring-opening reaction of the thiosuccinimide group was conducted using a method similar to that described in WO2015165413A1.

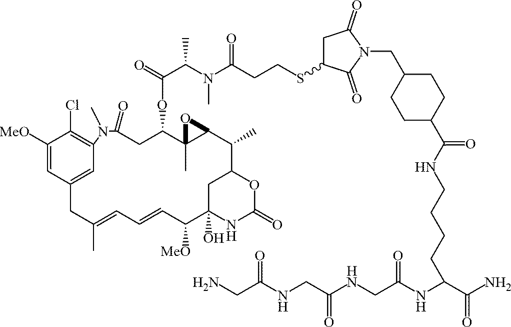 [SMILES: COc1cc2cc(c1Cl)N(C)C(=O)C[C@H](OC(=O)[C@H](C)N(C)C(=O)CCSC1CC(=O)N(CC3CCC(C(=O)NCCCCC(NC(=O)CNC(=O)CNC(=O)CN)C(N)=O)CC3)C1=O)[C@]1(C)O[C@H]1[C@H](C)C1C[C@@](O)(NC(=O)O1)[C@H](OC)/C=C/C=C(\C)C2]

Four isomers (isomeric compounds (i-1), (ii-1), (iii-1) and (iv-1)) are obtained from the ring-opening reaction. According to the difference of thiol group on α position and β position of the unbroken amide bond, they are named as α isomer and β isomer respectively. The structure of the four isomers are as follows:

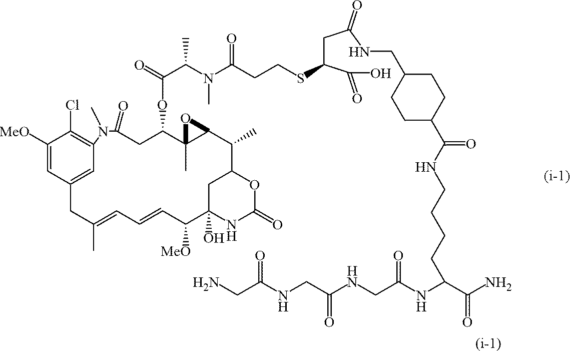

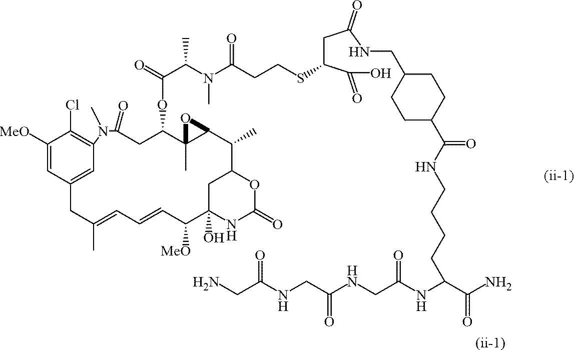

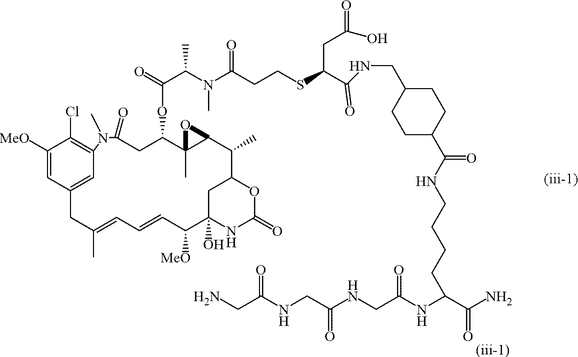

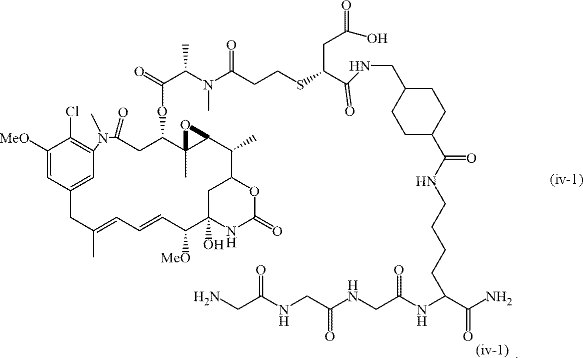

The reaction mixture resulting from the reaction which contains the four isomers was directly subjected to the process of separation as described below.

### Example 1: Separation of Β1, Β2, and Α Isomers

### 1.1 The Process for Separation

Gradient elution is applied for the separation of β1, β2, and α isomers. The eluotropic gradient includes both pH gradient and the organic solvent gradient. The chromatographic conditions are according to the Table 1:

TABLE 1

Column: Kromasil 100-10C18, 21.2*250 mm (100 Å, 10 µm)

| column temperature: r.t. (20-30° C.) | loading:≤550 mg | |
|---|---|---|
| Eluent A: 0.3% CH₃COOH in water | | |
| Eluent B: MeOH | | |
| flow rate: 12 mL/min | | |
| Eluotropic gradient (separation phase) | | |
| Time(min) | A% | B% |
| 10.1 | 57 | 43 |
| 90.0 | 47 | 53 |

Three major peaks are shown in the graph, namely Peak 1 (the first eluted peak in the range of about t_{R} 27 min to about t_{R} 39 min), Peak 2 (the second eluted peak in the range of about t_{R} 27 min to about t_{R} 39 min) and Peak 3 (the peak in the range of t_{R} 42 min to about t_{R} 51 min). The purity of each fraction of eluate was detected by HPLC using the analytical method as described in Example 2. The fractions were combined according to the analytical results, and then lyophilized and subjected to NMR assays. According to 1D and 2D NMR data, Peak 1 and Peak 2 contain two β isomers, respectively. The structure of β isomers are as follows:

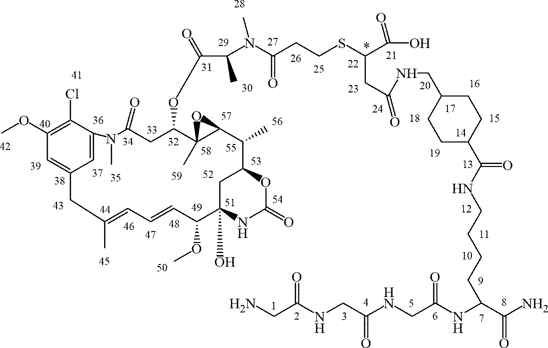

wherein ^{∗} denotes an asymmetric center (22-C).

The β isomer contained in Peak 1 is denoted as β1. ¹HNMR, ¹³C NMR, ¹H-¹H COSY and HMBC data are shown in Table 2.

TABLE 2

600 MHz NMR Assignments for isomer β1 in DMSO-d6

| Entry | C No. | ¹H NMR | ¹³C NMR | ¹H-¹H COSY | HMBC |
|---|---|---|---|---|---|
| 1 | 59 | 0.71, s, 3H | 11.84 | / | 32-H |
| 2 | 30 | 1.10,d, 3H,J=6.3 | 13.55 | 29-H | 29-H |
| 3 | 56 | 1.06,d, 3H, J=6.6 | 14.89 | 55-H | 57-H,55-H |
| 4 | 45 | 1.53,s,3H | 15.53 | 46-H | 46-H,43-H |
| 5 | 10 | 1.18,m,2H | 23.25 | 12-H,11-H | / |
| 6 | 25 | 2.58,2.75, m,2H | 25.65 | 26-H | 22-H,26-H |
| 7 | 11 | 1.60,m,2H | 29.29 | 10-H | / |
| 8 | 15,19 | 1.18, 1.27,m, 2H | 29.30^{∗}2 | 16-H,18-H,14-H | / |
| 9 | 16,18 | 0.73,1.61,m 2H | 30.02^{∗}2 | 14-H,15-H,16-H,18-H,19-H | 20-H |
| 10 | 28 | 2.63,s,3H | 30.18 | 29-H | 29-H |
| 11 | 9 | 1.45,1.60,2H | 31.91 | 7-H | 7-H |
| 12 | 33 | 1.98,2.45, m,2H | 32.39 | 33-H,32-H | 32-H |
| 13 | 26 | 2.34, 2.71,m, 2H | 34.26 | 26-H,25-H | 25-H |
| 14 | 35 | 3.03, s, 3H | 35.62 | / | / |
| 15 | 52 | 1.19, 1.40, m,2H | 36.8 | 53-H | 51-NH,49-H |
| 16 | 23 | 2.13, 2.58, dd, 2H | 37.26 | 22-H | 22-H |
| 17 | 17 | 1.20,m,1H | 37.56 | 16-H,18-H | 20-H,15-H,16-H,18-H,19-H |
| 18 | 55 | 1.39,m,1H | 38.15 | 56-H,57-H | 56-H |
| 19 | 12 | 2.91,m, 2H | 38.58 | 12-NH,10-H | 12-NH |
| 2 | 3,5 | 3.62, 3.70,m,2H | 42.57^{∗}2 | 6-NH | / |
| 21 | 22 | 3.43,m,1H | 42.66 | 23-H | 23-H |
| 22 | 1 | 3.23,s,2H | 43.71 | 1-H | / |
| 23 | 14 | 1.91,t,1H | 44.54 | 15-H,16-H | 16-H,18-H |
| 24 | 20 | 2.75, 2.80,m, 2H | 45.32 | 20-NH | 16-H,18-H |
| 25 | 43 | 3.13, 3.41, d, 2H | 45.97 | 43-H | 45-H,39-H,46-H |
| 26 | 29 | 5.24, q,1H,J=6.8 | 52.14 | 30-H,28-H | 30-H,28-H |
| 27 | 7 | 4.05,m, 1H | 52.82 | 6-NH,9-H | 6-NH |
| 28 | 50 | 3.18, s,3H | 56.6 | / | 49-H |
| 29 | 42 | 3.86, s,3H | 57.01 | / | / |
| 30 | 58 | / | 60.5 | / | 59-H,32-H |
| 31 | 57 | 2.72, d, 1H | 67.24 | 55-H | 59-H,56-H,55-H |
| 32 | 53 | 4.00,t, 1H | 73.64 | 52-H | 52-H,56-H |
| 33 | 32 | 4.46, dd, 1H,J=12,2.1 | 78.13 | 33-H | 59-H,33-H,57-H |
| 34 | 51 | / | 80.47 | / | 49-H,52-H |
| 35 | 49 | 3.43,m,1H | 88.66 | 48-H | 50-H,51-NH,47-H |
| 36 | 39 | 7.11,s,1H | 114.45 | 37-H | 37-H,43-H |
| 37 | 41 | / | 117.64 | / | 39-H,37-H |
| 38 | 37 | 6.48,d,1H | 122.11 | 39-H | 43-H,39-H |
| 39 | 46 | 6.51,d,1H | 125.63 | 47-H,48-H,45-H | 45-H,43-H,48-H |
| 40 | 48 | 5.50,dd,1H,J=13.9,9.1 | 128.96 | 46-H,47-H,49-H | 46-H,49-H |
| 41 | 47 | 6.50,dd,1H | 133.08 | 48-H,46-H | 49-H |
| 42 | 44 | / | 138.86 | / | 45-H |
| 43 | 36,38 | / | 141.75^{∗}2 | / | 35-H,37-H |
| 44 | 54 | / | 151.74 | / | 51-NH |
| 45 | 40 | / | 155.77 | / | 42-H,39-H |
| 46 | 34 | / | 168.63 | / | 33-H,35-H |
| 47 | 4 | / | 169.07 | / | 4-NH |
| 48 | 6 | / | 169.73 | / | 6-NH |
| 49 | 24 | / | 170.97 | / | 23-H |
| 50 | 31 | / | 171.07 | / | 32-H,29-H.30-H |
| 51 | 21 | / | 171.86 | / | 20-NH |
| 52 | 27 | / | 172.56 | / | 26-H |
| 53 | 2,8 | / | 174.14 | / | 8-NH |
| 54 | 13 | / | 175.43 | / | 12-NH,12-H |
| 55 | 6-NH | 7.79,d,1H,J=8.0 | / | 7-H,5-H | / |
| 56 | 20-NH | 7.93,t,1H,J=6.0 | / | 20-H | / |
| 57 | 4-NH | 8.19,br.s, 1H | / | 3-H,5-H | / |
| 58 | 2-NH, | 8.39,br.s, 1H | / | 3-H | / |
| 59 | 12-NH | 7.58,t,1H,J=6.0 | / | 12-H | / |
| 60 | 51-NH | 6.84,br.s, 1H | / | / | / |
| 61 | 8-NH | 6.97,7.22,S,2H | / | / | / |

The β isomer contained in Peak 2 is denoted as β2.

¹HNMR, ¹³C NMR, ¹H-¹H COSY and HMBC data are shown in table 3.

TABLE 3

600 MHz NMR Assignments for isomer β2 in DMSO-d6

| Entry | C No. | ¹H NMR | ¹³C NMR | ¹H-¹H COSY | HMB C |
|---|---|---|---|---|---|
| 1 | 59 | 0.71, s, 3H | 11.85 | / | 32-H |
| 2 | 30 | 1.10,d, 3H,J=6.3 | 13.59 | 29-H | 29-H |
| 3 | 56 | 1.06,d, 3H, J=6.6 | 14.89 | 55-H | 57-H,55-H |
| 4 | 45 | 1.53,s,3H | 15.52 | 46-H | 46-H,43-H |
| 5 | 10 | 1.17,m,2H | 23.27 | 12-H,11-H | / |
| 6 | 25 | 2.60,2.76, m,2H | 25.73 | 26-H | 22-H,26-H |
| 7 | 11 | 1.60,m,2H | 29.31 | 10-H | / |
| 8 | 15,19 | 1.18, 1.27,m, 2H | 29.31^{∗}2 | 16-H,18-H,14-H | / |
| 9 | 16,18 | 0.73,1.61,m 2H | 29.98^{∗}2 | 14-H,15-H,16-H,18-H,19-H | 20-H |
| 10 | 28 | 2.63,s,3H | 30.21 | 29-H | 29-H |
| 11 | 9 | 1.45,1.60,2H | 31.93 | 7-H | 7-H |
| 12 | 33 | 1.96,2.45, m,2H | 32.39 | 33-H,32-H | 32-H |
| 13 | 26 | 2.34, 2.67,m, 2H | 34.21 | 26-H,25-H | 25-H |
| 14 | 35 | 3.03, s, 3H | 35.60 | / | / |
| 15 | 52 | 1.19, 1.40, m,2H | 36.81 | 53-H | 51-NH,49-H |
| 16 | 23 | 2.28, 2.61, dd, 2H | 37.52 | 22-H | 22-H |
| 17 | 17 | 1.20,1H | 37.52 | 16-H,18-H | 20-H,15-H,16-H,18-H,19-H |
| 18 | 55 | 1.39,1H | 38.15 | 56-H,57-H | 56-H |
| 19 | 12 | 2.91,m, 2H | 38.59 | 12-NH,10-H | 12-NH |
| 2 | 3,5 | 3.62, 3.70,2H | 42.55^{∗}2 | 6-NH | / |
| 21 | 22 | 3.43,1H | 42.99 | 23-H | 23-H |
| 22 | 1 | 3.23,s,2H | 43.79 | 1-H | / |
| 23 | 14 | 1.91,t,1H | 44.53 | 15-H,16-H | 16-H,18-H |
| 24 | 20 | 2.65, 2.75,m, 2H | 45.27 | 20-NH | 16-H,18-H |
| 25 | 43 | 3.13, 3.41, d, 2H | 45.97 | 43-H | 45-H,39-H,46-H |
| 26 | 29 | 5.23, q,1H,J=6.8 | 52.15 | 30-H.28-H | 30-H,28-H |
| 27 | 7 | 4.06,m, 1H | 52.81 | 6-NH,9-H | 6-NH |
| 28 | 50 | 3.18, s,3H | 56.60 | / | 49-H |
| 29 | 42 | 3.86, s,3H | 56.99 | / | / |
| 30 | 58 | / | 60.52 | / | 59-H,32-H |
| 31 | 57 | 2.72, d, 1H | 67.20 | 55-H | 59-H,56-H,55-H |
| 32 | 53 | 4.00,t, 1H,J=12,2.1 | 73.62 | 52-H | 52-H,56-H |
| 33 | 32 | 4.55, dd, 1H | 78.09 | 33-H | 59-H,33-H,57-H |
| 34 | 51 | / | 80.47 | / | 49-H,52-H |
| 35 | 49 | 3.43,1H | 88.67 | 48-H | 50-H,51-NH,47-H |
| 36 | 39 | 7.11,s,1H | 114.39 | 37-H | 37-H,43-H |
| 37 | 41 | / | 117.61 | / | 39-H,37-H |
| 38 | 37 | 6.48,1H | 122.08 | 39-H | 43-H,39-H |
| 39 | 46 | 6.50,1H | 125.63 | 47-H,48-H,45-H | 45-H,43-H,48-H |
| 40 | 48 | 5.50,dd,1H,J=13.9,9.1 | 128.99 | 46-H,47-H,49-H | 46-H,49-H |
| 41 | 47 | 6.48,dd,1H | 133.08 | 48-H,46-H | 49-H |
| 42 | 44 | / | 138.84 | / | 45-H |
| 43 | 36,38 | / | 141.76^{∗}2 | / | 35-H,37-H |
| 44 | 54 | / | 151.73 | / | 51-NH |
| 45 | 40 | / | 155.72 | / | 42-H,39-H |
| 46 | 34 | / | 168.60 | / | 33-H,35-H |
| 47 | 4 | / | 169.06 | / | 4-NH |
| 48 | 6 | / | 169.72 | / | 6-NH |
| 49 | 24 | / | 170.92 | / | 23-H |
| 50 | 31 | / | 171.04 | / | 32-H,29-H.30-H |
| 51 | 21 | / | 171.77 | / | 20-NH |
| 52 | 27 | / | 172.64 | / | 26-H |
| 53 | 2,8 | / | 174.13 | / | 8-NH |
| 54 | 13 | / | 175.40 | / | 12-NH,12-H |
| 55 | 6-NH | 7.79,d,1H,J=8.0 | / | 7-H,5-H | / |
| 56 | 20-NH | 7.93,t,1H,J=6.0 | / | 20-H | / |
| 57 | 4-NH | 8.19,br.s, 1H | / | 3-H,5-H | / |
| 58 | 2-NH, | 8.39,br.s, 1H | / | 3-H | / |
| 59 | 12-NH | 7.58,t,1H,J=6.0 | / | 12-H | / |
| 60 | 51-NH | 6.84,br.s, 1H | / | / | / |
| 61 | 8-NH | 6.97,7.22,S,2H | / | / | / |

According to 1D and 2D NMR data, Peak 3 contains a isomer(s). We infer the contents of Peak 3 from the mechanism of the ring-opening reaction, and denote this peak as a mixture of two α isomers, namely α1 and α2.

Separation under other chromatographic conditions are tested and the results are listed in the following points 1.2 to 1.6.

### 1.2 Stationary Phase

Packed columns by different manufacturers are tested. The chromatographic conditions are according to Table 4.

TABLE 4

Tests using different columns

| Instrument | Agilent HPLC 1200 | | |
|---|---|---|---|
| Column | column 1:Daisopak C4 SP-120-8-C4-P (120 Å, 8 µm) | | |
| | column 2:Kromasil C18 100-10C18 (100 Å, 10 µm) | | |
| | column 3:Acchrom C18 C18-ME (100 Å, 10 µm) | | |
| | column 4:Daisopak C18 100-10-ODS-P (100 Å, 10 µm) | | |
| Column temperature | 20~30° C. (r.t.) | | |
| Injection volume | 5 µL | | |
| Flow rate | 1 mL/min | | |
| Wave length | 215 nm | | |
| Eluent A | 0.05%L-TA in water | | |
| Eluent B | MeOH | | |
| Gradient (separation phase) | Time (min) | A% | B% |
| | 0 | 56 | 44 |
| | 30 | 56 | 44 |

The HPLC graphs are as shown in FIGS. 1 to 4:

The purity of each fraction of eluate was detected by HPLC using the analytical method as described in Example 2. Three major peaks are shown in the graph. The fractions were combined according to the analytical results, and then lyophilized and subjected to NMR assays. According to the analytical results and 1D and 2D NMR data, the isomers were eluted in the same consequence as in Example 1 under the chromatographic conditions listed above. In FIGS. 2 to 4, the two β isomers are eluted in the first two peaks, respectively, and the mixture of two α isomers is eluted in the third peak.

The factors are calculated and listed in Table 5:

TABLE 5

| column | resolution | tailing factor | Theoretical plate number |
|---|---|---|---|
| column 1: Daisopak C4 SP-120-8-C4-P | ND | ND | ND |
| column 2: Kromasil C18 100-10C18 | 1.55 | 2.72 | 1197 |
| column 3: Acchrom C18 C18-ME 10 um 100 Å | 1.22 | 2.91 | 938 |
| column 4: Daisopak C18 100-10-ODS-P | 1.51 | 2.09 | 1500 |

On all the types of RP-C18 stationary phase, R greater than 1 is observed. The process of the present disclosure is suitable for use on different commercial packed columns and therefore possesses robustness.

### 1.3 Acid As Acid Agent 1

Different acids are tested for use as the Acid agent 1 in Eluent A. The chromatographic conditions are according to table 6.

TABLE 6

| Instrument | | Agilent HPLC 1200 | |
|---|---|---|---|
| Column | | Kromasil C18 100-10C18 | |
| Column temperature | 20~30° C. (r.t.) | | |
| Injection volume | 5 µL | | |
| Flow rate | 1 mL/min | | |
| Eluent A | 0.1% TFA in water | | |
| | 0.1% H₃PO₄ in water | | |
| | 0.2% TFA in water | | |
| | 0.1% CH₂O₂ in water | | |
| | 0.2% CH₂O₂ in water | | |
| | 0.5% AA | | |
| | 0.1% CH₃COOH in water | | |
| | 0.3% CH₃COOH in water | | |
| | 0.3% CH₃COOH+0.05% L-TA in water | | |
| Eluent B | MeOH | | |
| Gradient (separation phase) | Time (min) | A% | B% |
| | 0 | 54 | 46 |
| | 30 | 34 | 66 |

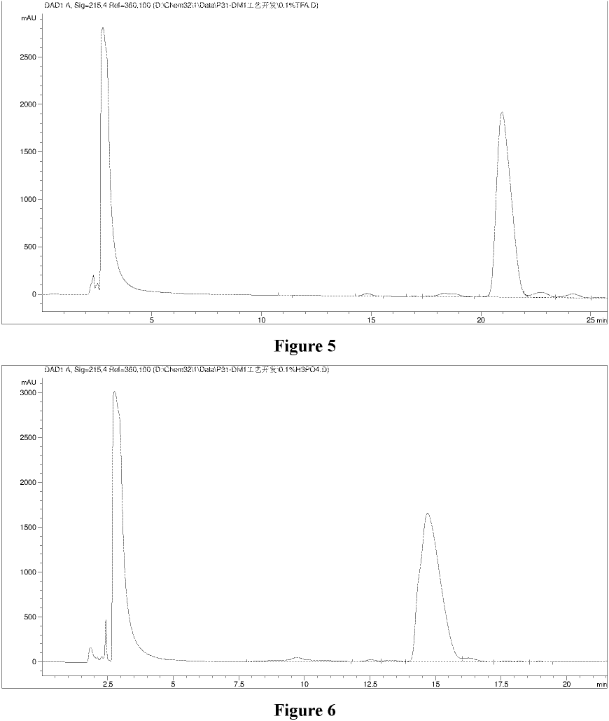

The HPLC graph of the separation is shown in FIGS. 5 to 13.

According to FIGS. 5, 7, 11 and 12, moderate separation is observed when using optionally substituted alkyl carboxylic acids, wherein AcOH and L-TA shows satisfactory resolution. Inorganic acid (e.g., phosphoric acid) also shows certain resolution. Formic acid demonstrates a poor resolution. The separation capability of the mobile phase may be influenced by the acid radical of the organic acid.

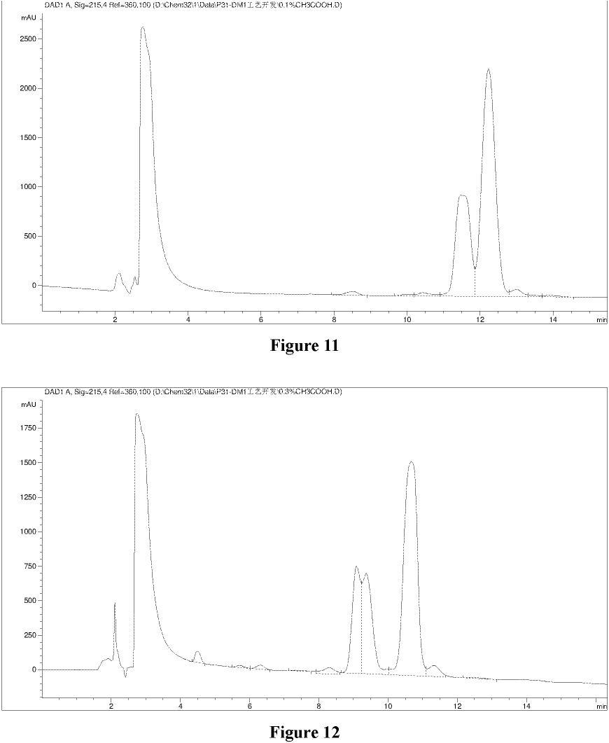

The purity of each fraction of eluate was detected by HPLC using the analytical method as described in Example 2. The fractions were combined according to the analytical results, and then lyophilized and subjected to NMR assays. According to the analytical results and 1D and 2D NMR data, the isomers were eluted in the same consequence as in Example 1 under the chromatographic conditions listed above. In FIGS. 11 to 13, the two β isomers are eluted in the first two peaks (partially merged with each other), and the mixture of two α isomers is eluted in the third peak.

### 1.4 Buffer Salt as Acid Agent 1

Phosphate buffers are tested for use as the Acid agent 1 in Eluent A.

Eluent A tested are according to table 7. The chromatographic conditions except for Eluent A are the same with those in table 6.

TABLE 7

| | |
|---|---|
| Eluent A | 10 mM NaH₂PO₄ PH=2.0 |
| | 10 mM NaH₂PO₄ PH=4.0 |
| | 10 mM NaH₂PO₄ PH=6.0 |
| | 10 mM K₂HPO₄ PH=4.0 |
| | 10 mM TEAP PH=4.0 |

The HPLC graph of the separation is shown in FIGS. 14 to 19.

Taking together the results in 1.3 and 1.4, it can be seen that pH value is not the only factor influencing the chromatographic behavior of the solutes. The separation of the isomers is significantly influenced by the species of the acidic agent(s) used. The applicant unexpectedly found that among the tested acidic agents, AcOH and L-TA exert significantly improved resolution as compared to the other acidic agents.

### 1.5 Organic Solvent

Analyzing the resolution between the two α isomers by comparison with that in Example 3, a much greater resolution is obtained when using MeOH as the organic solvent, indicating that the species of organic solvent may be a key factor.

### 1.6 Gradient

Different gradients are used in the above 1.1, 1.3 and 1.4, and the above 1.2 uses isocratic elution, indicating that the process of the present disclosure can tolerate a range of eluotropic compositions. This brings flexibility to the process and could be particularly beneficial in the pilot scale manufacturing and would help to relieve the pressure on the robustness control of the eluotropic compositions.

Further tests are conducted using 0.3% CH₃COOH in water as Eluent A and using the eluotropic gradients according to table 8-1 and 8-2. The chromatographic conditions except for Eluent A and eluotropic gradients are the same with those in table 6.

TABLE 8-1

| Eluent A | 0.3% CH₃COOH in water | | |
|---|---|---|---|
| Gradient 1 (separation phase) | Time (min) | A% | B% |
| | 0 | 54 | 46 |
| | 30 | 34 | 66 |

TABLE 8-2

| Eluent A | 0.3% CH₃COOH in water | | |
|---|---|---|---|
| Gradient 2 (separation phase) | Time (min) | A% | B% |
| | 0 | 54 | 46 |
| | 80 | 44 | 56 |

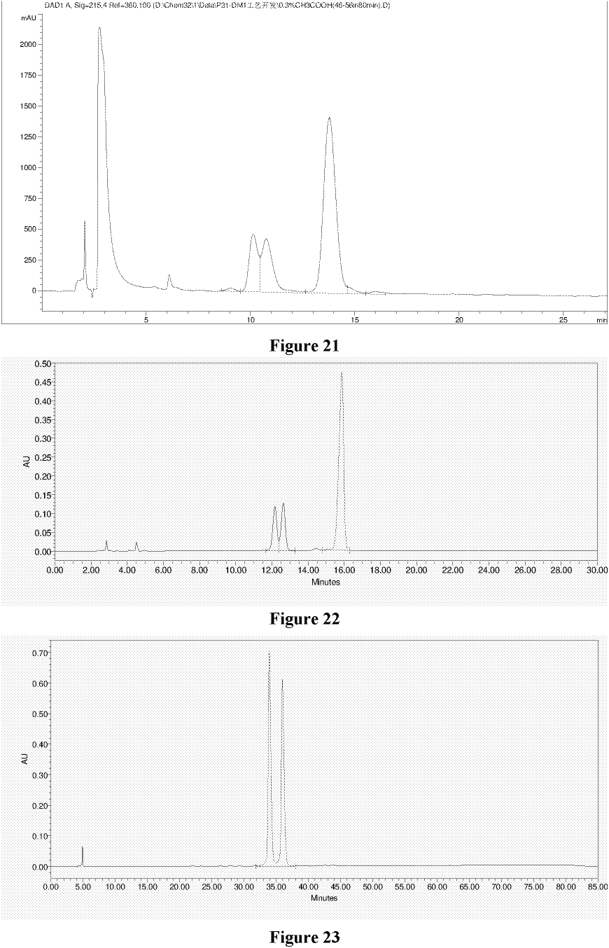

The HPLC graph of the separation is shown in FIGS. 20 to 21.

The purity of each fraction of eluate was detected by HPLC using the analytical method as described in Example 2. The fractions were combined according to the analytical results, and then lyophilized and subjected to NMR assays. According to the analytical results and 1D and 2D NMR data, the isomers were eluted in the same consequence as in Example 1 under the chromatographic conditions listed above. In FIGS. 20 to 21, the two β isomers are eluted in the first two peaks (partially merged with each other), and the mixture of two α isomers is eluted in the third peak.

The factors are calculated and listed in Table 9:

TABLE 9

| | | | |
|---|---|---|---|
| Gradient | Resolution | Tailing factor | Theoretical plate number |
| Gradient 1 | 2.11 | 0.91 | 3479 |
| (46%-66% B in 30 min) | | | |
| Gradient 2 | 2.97 | 1.12 | 2622 |
| 46%-56% B in 80 min | | | |

The gradient of 46%-56% B over 80 min achieves better resolution than the gradient of 46%-66% B over 30 min. Given the increase in resolution, the tailing factor (increased) and the theoretical plate number (decreased) of Gradient 2 are still acceptable.

Further prolonged elution than 80 min shows unsatisfactory results in system suitability tests.

### Example 2: In-Process Control for the Separation of Β1, Β2, and Α Isomers

In Example 2, the process described in Example 1 is applied in analytical scale using a stationary phase with smaller particle size (5 um) than that used in Example 1 (10 um). The chromatographic conditions are according to Table 10.

TABLE 10

| Column | | Phenomenex Gemini 110 Å 5 um 250^{∗}4.6 mm | |
|---|---|---|---|
| Column temperature | | 35° C. | |
| Injection volume | | injection volume : 5~ 100 µL | |
| Flow rate | | 0.7 mL/min | |
| Eluent A | | 0.3% CH₃COOH in water | |
| Eluent B | MeOH | | |
| Gradient (separation phase) | Time (min) | A% | B% |
| | 0 | 58 | 42 |
| | 30 | 38 | 62 |

The HPLC graph of the separation is shown in FIG. 22.

The resolution is calculated to be 1.28, and the theoretical plate number is 20020.6.

### Example 3: Separation of Α1 and Α2 Isomers

### 3.1 The Process for Separation

The α isomers obtained in Example 1 are subjected to further separation to gain isomer α1 and isomer α2. The structure of α isomers are as follows:

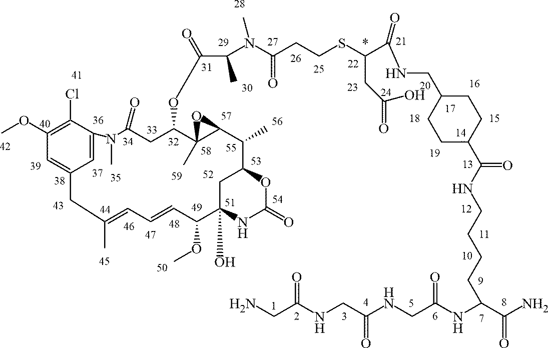

wherein ^{∗} denotes an asymmetric center (22-C).

Gradient elution is applied for the separation of α1 and α2 isomers from the α isomers obtained in Example 1. The chromatographic conditions are according to Table 11.

TABLE 11

Column: Kromasil 100-10C18, 21.2^{∗}250 mm (100 Å, 10 µm)

| column temperature: r.t. (20-30° C.) | | | loading:≤200 mg |
|---|---|---|---|
| Eluent C: 0.1% TFA in water | | | |
| Eluent D: 0.1% TFA in ACN | | | |
| flow rate: 12 mL/min | | | |
| Eluotropic gradient (separation phase) | | | |
| Time(min) | A% | B% | |
| 5.1 | 77 | 23 | |
| 85.0 | 67 | 33 | |

The HPLC graph of the separation is shown in FIG. 23. Two major peaks are shown in the graph, namely Peak 1′ (t_{R} 40.075 min) and Peak 2′ (t_{R} 42.497 min). The purity of each fraction of eluate was detected by HPLC using the analytical method as described in Example 4. The α isomer contained in Peak 1 is denoted as α1. ¹H NMR, ¹³C NMR, ¹H-¹H COSY and HMBC data are shown in table 12.

TABLE 12

600 MHz NMR Assignments for isomer α1 in DMSO-d6

| Entry | C No. | ¹H NMR | ¹³C NMR | ¹H-¹H COSY | HMBC |
|---|---|---|---|---|---|
| 1 | 59 | 0.71, s, 3H | 11.83 | / | 32-H |
| 2 | 30 | 1.10,d, 3H,J=6.3 | 13.53 | 29-H | 29-H |
| 3 | 56 | 1.06,d, 3H, J=6.7 | 14.89 | 55-H | 57-H,55-H |
| 4 | 45 | 1.53,s,3H | 15.54 | 46-H | 46-H,43-H |
| 5 | 10 | 1.18,m,2H | 23.21 | 12-H,11-H | 7-H,12-H,9-H |
| 6 | 25 | 2.58,2.75, m,2H | 26.65 | 26-H | 22-H,26-H |
| 7 | 11 | 1.60,m,2H | 29.27 | 10-H | 12-H |
| 8 | 15,19 | 1.18, 1.27,m, 2H | 29.27^{∗}2 | 16-H,18-H,14-H | 17-H |
| 9 | 16,18 | 0.73,1.61,m 2H | 30.07^{∗}2 | 14-H,15-H,16-H,18-H,19-H | 20-H |
| 10 | 28 | 2.64,s,3H | 30.19 | 29-H | 29-H |
| 11 | 9 | 1.45,1.60,m,2H | 31.90 | 7-H | 7-H,6-NH,10-H |
| 12 | 33 | 1.98, 2.45, m,2H | 32.40 | 33-H,32-H | 32-H |
| 13 | 26 | 2.35, 2.74,m, 2H | 34.01 | 26-H,25-H | 25-H |
| 14 | 35 | 3.03, s, 3H | 35.66 | / | / |
| 15 | 52 | 1.19, 1.40, m,2H | 36.81 | 53-H | 51-NH,49-H,53-H |
| 16 | 17 | 1.20,m,1H | 37.53 | 16-H,18-H | 20-H,15-H,16-H,18-H,19-H |
| 17 | 55 | 1.39,m,1H | 38.16 | 56-H,57-H | 56-H,57-H |
| 18 | 23 | 2.00, 2.42, dd, 2H | 38.20 | 22-H | 22-H |
| 19 | 12 | 2.91,m, 2H | 38.53 | 12-NH,10-H | 12-NH |
| 2 | 3,5 | 3.63, 3.69,m,2H | 42.58^{∗}2 | 6-NH | 2-NH,4-NH |
| 21 | 1 | 3.28,s,2H | 43.02 | 1-H | / |
| 22 | 22 | 3.31,m,1H | 43.41 | 23-H | 23-H |
| 23 | 14 | 1.91,t,1H | 44.55 | 15-H,16-H | 16-H,18-H |
| 24 | 20 | 2.72, 2.80,m,2H | 45.15 | 20-NH | 16-H,18-H |
| 25 | 43 | 3.13, 3.40, d, 2H | 45.98 | 43-H | 45-H,39-H,46-H |
| 26 | 29 | 5.24, q,1H,J=6.8 | 52.17 | 30-H.28-H | 30-H,28-H |
| 27 | 7 | 4.05,m, 1H | 52.82 | 6-NH,9-H | 6-NH |
| 28 | 50 | 3.18, s,3H | 56.60 | / | 49-H |
| 29 | 42 | 3.86, s,3H | 56.98 | / | / |
| 30 | 58 | / | 60.50 | / | 59-H,32-H |
| 31 | 57 | 2.72, d, 1H | 67.25 | 55-H | 59-H,56-H,55-H |
| 32 | 53 | 4.00,t, 1H | 73.64 | 52-H | 52-H,56-H |
| 33 | 32 | 4.45, dd, 1H,J=12,2.1 | 78.13 | 33-H | 59-H,33-H,57-H |
| 34 | 51 | / | 80.47 | / | 49-H,52-H |
| 35 | 49 | 3.43,d,1H | 88.67 | 48-H | 50-H,51-NH,47-H |
| 36 | 39 | 7.10,s,1H | 114.41 | 37-H | 37-H,43-H |
| 37 | 41 | / | 117.63 | / | 39-H,37-H |
| 38 | 37 | 6.48,s,1H | 122.15 | 39-H | 43-H,39-H |
| 39 | 46 | 6.51,dd,1H | 125.62 | 47-H,48-H,45-H | 45-H,43-H,48-H |
| 40 | 48 | 5.50,dd,1H,J=13.9,9.1 | 128.96 | 46-H,47-H,49-H | 46-H,49-H |
| 41 | 47 | 6.50,dd,1H | 133.08 | 48-H,46-H | 49-H |
| 42 | 44 | / | 138.88 | / | 45-H |
| 43 | 36,38 | / | 141.67, 141.77 | / | 35-H,37-H |
| 44 | 54 | / | 151.73 | / | 51-NH |
| 45 | 40 | / | 155.73 | / | 42-H,39-H |
| 46 | 34 | / | 168.61 | / | 33-H,35-H |
| 47 | 4 | / | 169.06 | / | 4-NH |
| 48 | 21 | / | 169.66 | / | 20-NH |
| 49 | 6 | / | 169.79 | / | 6-NH |
| 50 | 27,31 | / | 170.97, 171.06 | / | 32-H,29-H,30-H |
| 51 | 24 | / | 173.84 | / | 23-H |
| 53 | 2, 8 | / | 174.16 | / | 1-H,7-H,8-NH2 |
| 54 | 13 | / | 175.46 | / | 12-NH,12-H |
| 55 | 6-NH | 7.80,d,1H,J=8.0 | / | 7-H,5-H | / |
| 56 | 20-NH | 7.77,t,1H,J=6.0 | / | 20-H | / |
| 57 | 4-NH | 8.23,br.s, 1H | / | 3-H,5-H | / |
| 58 | 2-NH, | 8.47,br.s, 1H | / | 3-H | / |
| 59 | 12-NH | 7.58,t,1H,J=6.0 | / | 12-H | / |
| 60 | 51-NH | 6.84,br.s, 1H | / | / | / |
| 61 | 8-NH₂ | 6.96,7.24,S,2H | / | / | / |

The α isomer contained in Peak 2 is denoted as α2. ¹H NMR, ¹³C NMR, ¹H-¹H COSY and HMBC data are shown in table 13.

TABLE 13

600 MHz NMR Assignments for isomer α2 in DMSO-d6

| Entry | C No. | ¹H NMR | ¹³C NMR | ¹H-¹H COSY | HMBC |
|---|---|---|---|---|---|
| 1 | 59 | 0.71, s, 3H | 11.85 | / | 32-H |
| 2 | 30 | 1.10,d, 3H,J=6.3 | 13.60 | 29-H | 29-H |
| 3 | 56 | 1.06,d, 3H, J=6.7 | 14.89 | 55-H | 57-H,55-H |
| 4 | 45 | 1.53,s,3H | 15.51 | 46-H | 46-H,43-H, |
| 5 | 10 | 1.18,m,2H | 23.22 | 12-H,11-H | 7-H,12-H,9-H |
| 6 | 25 | 2.65,2.80, m,2H | 26.75 | 26-H | 22-H,26-H |
| 7 | 11 | 1.60,m,2H | 29.29 | 10-H | 12-H |
| 8 | 15,19 | 1.18, 1.27,m, 2H | 29.29^{∗}2 | 16-H,18-H,14-H | 17-H |
| 9 | 16,18 | 0.74,1.61,m 2H | 30.07^{∗}2 | 14-H,15-H,16-H,18-H,19-H | 20-H |
| 10 | 28 | 2.64,s,3H | 30.23 | 29-H | 29-H |
| 11 | 9 | 1.44,1.59,2H | 31.93 | 7-H | 7-H,6-NH,10-H |
| 12 | 33 | 1.97,2.45, m,2H | 32.41 | 33-H,32-H | 32-H |
| 13 | 26 | 2.39, 2.72,m, 2H | 34.08 | 26-H,25-H | 25-H |
| 14 | 35 | 3.03, s, 3H | 35.64 | / | / |
| 15 | 52 | 1.19, 1.40, m,2H | 36.82 | 53-H | 51-NH,49-H,53-H |
| 16 | 17 | 1.20,m,1H | 37.52 | 16-H,18-H | 20-H,15-H,16-H,18-H,19-H |
| 17 | 55 | 1.39,m,1H | 38.16 | 56-H,57-H | 56-H,57-H |
| 18 | 23 | 2.00, 2.42, dd, 2H | 38.40 | 22-H | 22-H |
| 19 | 12 | 2.91,m, 2H | 38.53 | 12-NH,10-H | 12-NH |
| 2 | 3,5 | 3.63, 3.69,m,2H | 42.54^{∗}2 | 6-NH | 2-NH,4-NH |
| 21 | 1 | 3.25,s,2H | 43.40 | 1-H | / |
| 22 | 22 | 3.33,1H | 43.77 | 23-H | 23-H |
| 23 | 14 | 1.91,t,1H | 44.55 | 15-H,16-H | 16-H,18-H |
| 24 | 20 | 2.75, 2.80,m, 2H | 45.16 | 20-NH | 16-H,18-H |
| 25 | 43 | 3.13, 3.37, d, 2H | 45.98 | 43-H | 45-H,39-H,46-H |
| 26 | 29 | 5.24, q,1H,J=6.8 | 52.12 | 30-H.28-H | 30-H,28-H |
| 27 | 7 | 4.06,m, 1H | 52.79 | 6-NH,9-H | 6-NH |
| 28 | 50 | 3.18, s,3H | 56.60 | / | 49-H |
| 29 | 42 | 3.86, s,3H | 57.00 | / | / |
| 30 | 58 | / | 60.52 | / | 59-H,32-H |
| 31 | 57 | 2.72, d, 1H | 67.19 | 55-H | 59-H,56-H,55-H |
| 32 | 53 | 4.02,t, 1H | 73.63 | 52-H | 52-H,56-H |
| 33 | 32 | 4.45, dd, 1H,J=12,2.1 | 78.07 | 33-H | 59-H,33-H,57-H |
| 34 | 51 | / | 80.47 | / | 49-H,52-H |
| 35 | 49 | 3.42,d,1H | 88.67 | 48-H | 50-H,51-NH,47-H |
| 36 | 39 | 7.10,s,1H | 114.40 | 37-H | 37-H,43-H |
| 37 | 41 | / | 117.61 | / | 39-H,37-H |
| 38 | 37 | 6.48,s,1H | 122.07 | 39-H | 43-H,39-H |
| 39 | 46 | 6.51,dd,1H | 125.63 | 47-H,48-H,45-H | 45-H,43-H,48-H |
| 40 | 48 | 5.50,dd,1H,J=13.9,9.1 | 129.01 | 46-H,47-H,49-H | 46-H,49-H |
| 41 | 47 | 6.50,dd,1H | 133.06 | 48-H,46-H | 49-H |
| 42 | 44 | / | 138.82 | / | 45-H |
| 43 | 36,38 | / | 141.70, 141.80 | / | 35-H,37-H |
| 44 | 54 | / | 151.72 | / | 51-NH |
| 45 | 40 | / | 155.71 | / | 42-H,39-H |
| 46 | 34 | / | 168.59 | / | 33-H,35-H |
| 47 | 4 | / | 169.03 | / | 4-NH |
| 48 | 21 | / | 169.66 | / | 20-NH |
| 49 | 6 | / | 169.82 | / | 6-NH |
| 50 | 27,31 | / | 171.05, 171.41 | / | 32-H,29-H.30-H |
| 51 | 24 | / | 173.72 | / | 23-H, |
| 53 | 2, 8 | / | 174.12 | / | 1-H,7-H,8-NH2 |
| 54 | 13 | / | 175.44 | / | 12-NH,12-H |
| 55 | 6-NH | 7.80,d,1H,J=8.0 | / | 7-H,5-H | / |
| 56 | 20-NH | 7.85,t,1H,J=6.0 | / | 20-H | / |
| 57 | 4-NH | 8.18,br.s, 1H | / | 3-H,5-H | / |
| 58 | 2-NH, | 8.40,br.s, 1H | / | 3-H | / |
| 59 | 12-NH | 7.57,t,1H,J=6.0 | / | 12-H | / |
| 60 | 51-NH | 6.83,br.s, 1H | / | / | / |
| 61 | 8-NH₂ | 6.96,7.22,S,2H | / | / | / |

Separation under other chromatographic conditions are tested and the results are listed in the following points 3.2 to 3.6.

### 3.2 Acid As Acid Agent 3

Different acids are tested for use as the Acid agent 3 in Eluent C. The chromatographic conditions are according to table 14.

TABLE 14

| Instrument | Agilent HPLC 1200 | | |
|---|---|---|---|
| Column | Kromasil C18 100-10C18 | | |
| Column temperature | 20~30° C.(r.t.) | | |
| Injection volume | 5 µL | | |
| Flow rate | 1 mL/min | | |
| Eluent C | 0.1%TFA in water | | |
| | 0.1% AcOH in water | | |
| | 0.3% AcOH in water | | |
| | 0.1% CH₂O₂ in water | | |
| | 0.3% CH₂O₂ in water | | |
| | 0.5% AA in water | | |
| | 0.1% H₃PO₄ in water | | |
| | 0.2% H₃PO₄ in water | | |
| Gradient (separation phase) | Time(min) | A% | B% |
| | 0 | 76 | 24 |
| | 30 | 56 | 44 |

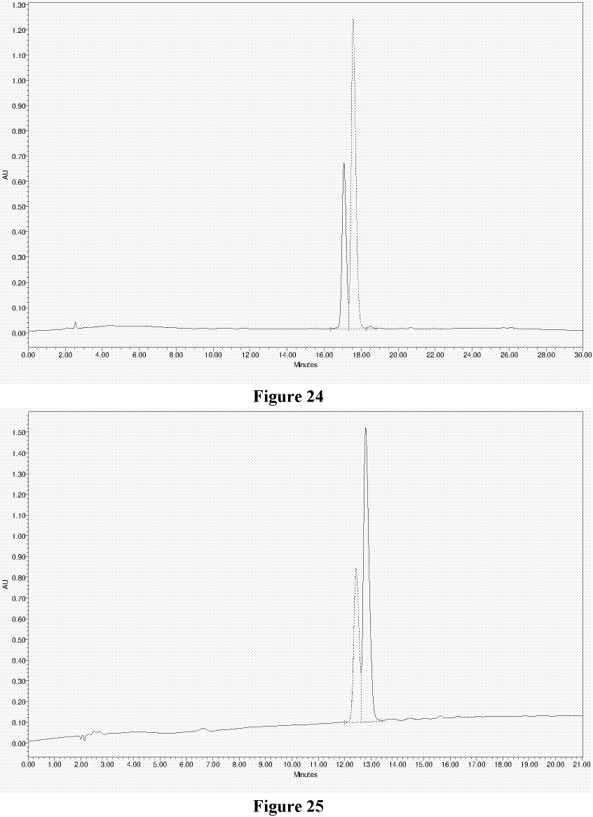

The HPLC graph of the separation is shown in FIGS. 24 to 31.

### 3.3 Buffer Salt As Acid Agent 3

Phosphate buffers are tested for use as the Acid agent 1 in Eluent A.

Eluent C tested are according to table 15. The chromatographic conditions except for Eluent C are the same with those in table 14.

TABLE 15

| | |
|---|---|
| Eluent C | 10 mM NaH₂PO₄ PH=2.0 |
| | 10 mM NaH₂PO₄ PH=4.0 |
| | 10 mM NaH₂PO₄ PH=6.0 |
| | 10 mM K₂HPO₄ PH=2.0 |
| | 10 mM K₂HPO₄ PH=4.0 |
| | 10 mM K₂HPO₄ PH=6.0 |
| | 10 mM K₂HPO₄ PH=8.0 |
| | 30 mM NaH₂PO₄ PH=2.0 |
| | 30 mM NaH₂PO₄ PH=6.0 |
| | 30 mM NaH₂PO₄ PH=4.0 |
| | 10 mM TEAP PH=6.0 |
| | 10 mM TEAP PH=2.0 |
| | 10 mM TEAP PH=4.0 |

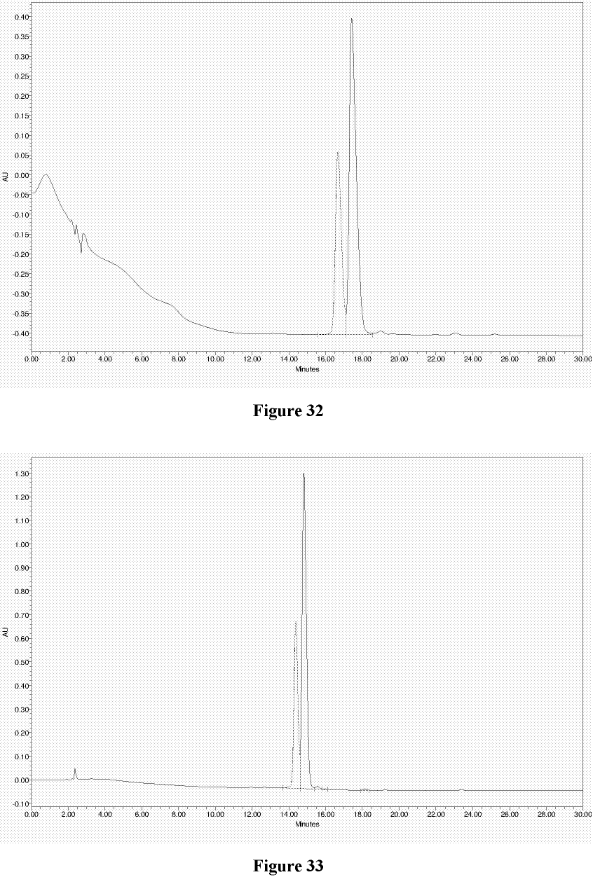

The HPLC graph of the separation is shown in FIGS. 32 to 44.

### 3.4 Summary of Acid Agent 3

The factors are calculated and listed in Table 16:

TABLE 16

| Acid agent 3 | Resolution | Acid agent 3 | Resolution |
|---|---|---|---|
| 0.1% TFA | 1.1277 | 10 mM NaH₂PO₄ PH=2.0 | 1.1493 |
| 0.1% AcOH | 0.9400 | 10 mM NaH₂PO₄ PH=4.0 | 1.0739 |
| 0.3% AcOH | ND^{∗} | 10 mM NaH₂PO₄ PH=6.0 | 1.0044 |
| 0.1% CH₂O₂ | 0.6984 | 10 mM K₂HPO₄ PH=2.0 | 1.1803 |
| 0.3% CH₂O₂ | 0.6768 | 10 mM K₂HPO₄ PH=4.0 | 1.1872 |
| 0.5% AA | 1.0227 | 10 mM K₂HPO₄ PH=6.0 | 1.0072 |
| 0.1% H₃PO₄ | 0.8875 | 10 mM K₂HPO₄ PH=8.0 | 0.9989 |
| 0.2% H₃PO₄ | 0.9731 | - | - |
| 30 mM NaH₂PO₄ PH=2.0 | 1.1929 | 10 mM TEAP PH=6.0 | 0.9914 |
| 30 mM NaH₂PO₄ PH=6.0 | 0.9612 | 10 mM TEAP PH=2.0 | 1.1617 |
| 30 mM NaH₂PO₄ PH=4.0 | 0.9958 | 10 mM TEAP PH=4.0 | 1.1576 |

^{∗}: Not Determined

Resolution of greater than 1 is achieved when 0.1% TFA, 10 mM K₂HPO₄ (PH=2.0), 10 mM K₂HPO₄ (PH=4.0), 10 mM TEAP (PH=2.0) or 10 mM TEAP PH=4.0 is used as Acid agent 3.

It has been shown in FIGS. 23-44 and Table 16 that the species of acidic agent exerts a less significant influence than in the process of Example 1, showing the difference between chromatographic characteristics of the β isomers and the α isomers.

### Example 4: In-Process Control for the Separation of Α1 and Α2 Isomers

In Example 4, the process described in Example 3 is applied in analytical scale using a stationary phase with smaller particle size (5 um) than that used in Example 3 (10 um). The chromatographic conditions are according to table 17.

TABLE 17

| Column | Waters X-select, CSH Phenyl-Hexyl 3.5um 150^{∗}4.6 mm | | |
|---|---|---|---|
| Column temperature | 35° C. | | |
| Injection volume | injection volume :5~100 µL | | |
| Flow rate | 0.7 mL/min | | |
| Eluent C | 10 mM NaH₂PO₄ in water, pH=2.0 | | |
| Eluent D | ACN | | |
| Gradient (separation phase) | Time (min) | A% | B% |
| | 0.0 | 76 | 24 |
| | 30.0 | 56 | 44 |

The HPLC graph of the analysis is shown in FIG. 45.

The analysis process of the present disclosure can be conducted using parameter sets as listed in any one of tables 17 to 19. The flexibility of the process facilitates its application under different conditions and could also take the detection requirement during different stages of the manufacture of the aimed product, such as the manufacture of the bioconjugate.

### Example 5: Cytotoxicity of Β Isomers, Α1 Isomer and Α2 Isomer

The peaks eluted in Example 1 were collected, wherein: (1) the first and the second peaks are collected as a sample (β isomers), and (2) the third peak is collected as a sample (α isomers) and subjected to separation using method as described in Example 3 and to obtain α1 isomer and α2 isomer.

The cytotoxicity of the β isomers, α1 isomer and α2 isomer are tested via tumor cell proliferation assay.

The detection methods:

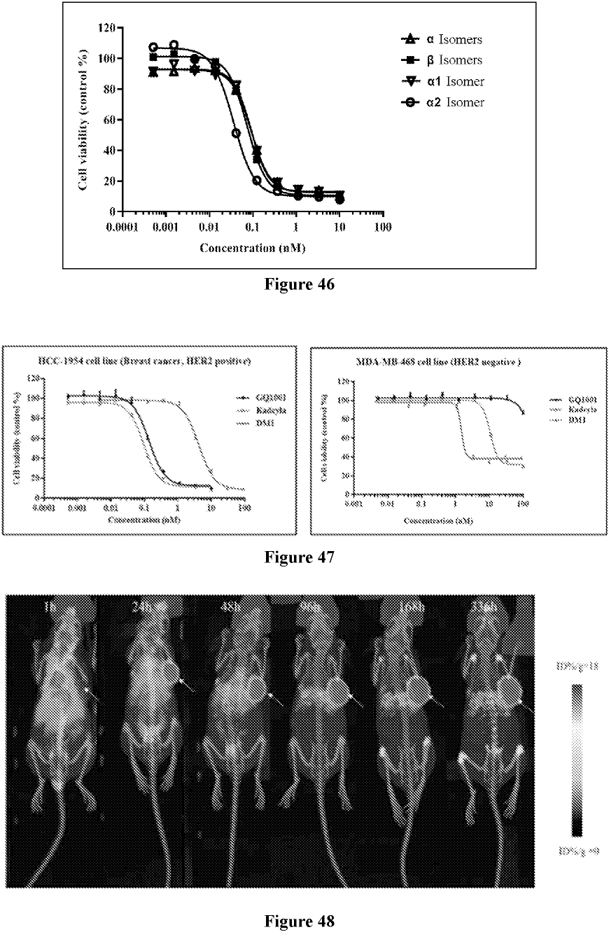

Human breast cancer HCC1954 cells in good growth condition with a cell fusion degree about 80% were digested by 0.25% trypsin, collected, placed in 96-well plates and cultured overnight. After cell adherence, the tested agents were diluted 3 times from 10 nM with a total of 10 gradients. After 72 h incubation at 37° C. in the incubator, cell viability was detected by CellTiter-Glo® luminescent kit. Cell vitality formula: Viability = (RLU (X) - RLU (Puro))/(RLU (Control) - RLU (Puro)) ^{∗} 100%. A logistic model (4-parameter) will be used for data processing, IC50 calculation and curve fitting by Prism 6 software. The result is shown in FIG. 46 and Table 20.

TABLE 20

Cytotoxicity of isomers in HER2 positive cell HCC1954

| Drug | IC₅₀ (nM) | Relative activity (%)^{∗} | R² |
|---|---|---|---|
| β isomers | 0.07399 | 100 | 0.9991 |
| α1 isomer | 0.09224 | 80.2 | 0.9976 |
| α2 isomer | 0.03601 | 205 | 0.9974 |

^{∗}: The biological activity of β isomers in HCC1954 cells is set as 100%.

As can be seen in Table 20, the activity of α1 isomer is within ±30% of the activity of β isomers, and therefore their activities are considered to be equivalent. The relative activity of isomer α2 is 205% (the biological activity of β isomers is set as 100%), namely, the activity of isomer α2 is about 2 fold stronger than β isomers.

### Example 6: Preparation of α-ADCs

The pure product containing α isomers (mixture of α1 and α2) obtained in Example 1 is subjected to conjugation reaction with therapeutic antibody. The method of the conjugation reaction is similar to that described in WO2015165413A1. The obtained product is a mixture of ADCs, denoted as “α-ADCs”. α-ADCs prepared by coupling α isomers with T-LCCT_{L}-HC as described in WO2015165413A1 (T represents Trastuzumab) is denoted as “α-ADC composition 1”.

### Example 7: HER2 Selective Cytotoxicity of HER2 Targeting α-ADCs

Objective: To evaluate the selective cytotoxicity of α-ADCs, HER2 positive cell HCC1954 and HER2 negative cell MDA-MB-468 were treated with α-ADC composition 1, Kadcyla and DM1, separately.

Study Design:

TABLE 21

Design of cytotoxicity in HER2 positive cell HCC1954

| Groups | Drug | First concentration(nM) | Gradient dilution ratio |
|---|---|---|---|
| | α-ADC | | |
| 1 | composition 1 | 10 | 3 |
| 2 | Kadcyla | 10 | 3 |
| 3 | DM1 | 90 | 3 |

TABLE 22

Design of cytotoxicity in HER2 negative cell MDA-MB-468

| Groups | Drug | First concentration(nM) | Gradient dilution ratio |
|---|---|---|---|
| | α-ADC | | |
| 1 | composition 1 | 100 | 3 |
| 2 | Kadcyla | 100 | 3 |
| 3 | DM1 | 90 | 3 |

Study design was shown as in Table 21 and Table 22. Cells were incubated with α-ADC composition 1, Kadcyla or DM1 for 72 h, and then cell viability was detected by CellTiter-Glo® luminescent kit. Cell vitality formula: Viability = (RLU (X) - RLU (Puro))/(RLU (Control) - RLU (Puro)) ^{∗} 100%. A logistic model (4-parameter) will be used for data processing, IC50 calculation and curve fitting by Prism 6 software.

Result: The results were shown in FIG. 47 and Table 23.

TABLE 23

The effects of α-ADCs on proliferation of cancer cells

| Test Article | IC₅₀ (nM) | |
|---|---|---|
| | HCC1954 | MDA-MB-468 |
| α-ADC composition 1 | 0.1361 | N/A |
| Kadcyla | 0.09472 | 10.55 |
| DM1 | 4.195 | 1.495 |

α-ADCs had significant inhibitory effect on the proliferation of HER2-positive cells, and its effect is tens of times better than DM1. T-DM1 (Kadcyla) had slightly stronger effect than α-ADCs, which result from that the toxin of T-DM1 is about twice that of α-ADCs (T-DM1 DAR≈3.5, α-ADC composition 1 DAR 1.79).

α-ADCs had no inhibitory effect up to 100 nM in HER2-negative cells, while showed significant inhibitory effect in HER2-positive cells. T-DM1 showed off-target cytotoxicity at high concentration in HER2 negative cells while α-ADCs not. This off-target cytotoxicity may be due to DM1 dropping off through Inverse Michael Reaction of MCC-DM1 in T-DM1. The MCC′ linker (comprising ring-opened MCC structure) of α-ADCs was designed to decrease Inverse Michael Reaction, which avoid or significantly reduce DM1 detachment without internalization. It demonstrates α-ADCs is superior safe to T-DM1 in normal cells.

### Example 8: Bio-Distribution Characteristics of α-ADCs

Objective: To investigate the bio-distribution characteristics of ⁸⁹Zr-α-ADCs in tumor-bearing mice by positron emission computed tomography/computed tomography (PET/CT) scanning at different time points after a single dose of intravenous injection of ⁸⁹Zr-α-ADC composition 1.

Study Design: [⁸⁹Zr] isotope labeling method was used to investigate the distribution of α-ADC composition 1 in BT-474 human breast cancer xenografts in athymic mice (referred to as BT-474 xenograft tumor model) after single intravenous injection. Qualified ⁸⁹Zr-α-ADC composition 1 was administered to experimental animals. 8 BT-474 xenograft tumor models were administered ⁸⁹Zr-α-ADC composition 1. PET/CT scan was performed at 1 h, 24 h, 48 h, 96 h, 168 h and 336 h for static scanning for 10 to 30 min after administration.

Result: Result was shown in FIG. 48.

After a single dose of intravenous injection of ⁸⁹Zr-α-ADC composition 1 in BT-474 xenograft tumor models, total radioactivity was mainly distributed in the tumor, followed by the blood-rich organs (liver, heart, kidney, spleen, lung) and Knee, and less in other tissues and organs (Bone, brain, muscle).The order of AUC(0-336 h) of ⁸⁹Zr-α-ADC composition 1 in different tissues calculated based on radioactivity was as follows: tumor > liver>Knee> heart> kidney > spleen >lung > Bone > muscle > brain. These results suggested that it was difficult to cross the blood-brain barrier and the tumor target was obvious of ⁸⁹Zr-α-ADC composition 1.

After a single intravenous injection of ⁸⁹Zr-α-ADC composition 1 in BT-474 xenograft tumor models, the ratio (tumor-to-muscle and tumor-to-heart) increased gradually, both reached the highest at 336 h post dose, which was 34.78 and 15.54 respectively.

### Example 9: In Vivo Anti-Tumor Efficacy of α-ADCs

Objective: The objective of this study was to evaluate the in vivo anti-tumor efficacy of α-ADCs after single dose IV administration in the subcutaneous HCC1954 human breast cancer xenograft model (HCC1954 model) and NCI-N87 human gastric cancer xenograft model (NCI-N87 model) in female BALB/c nude mice.

Study Design: Study design was shown in Table 24 and Table 25.

TABLE 24

Study design in HCC1954 model

| Group | N | Treatment | Dose (mg/kg) | Dosing Volume (µL/g)^{b} | Dosing Route | Schedule^{c} |
|---|---|---|---|---|---|---|
| 1 | 8 | Vehicle | -- | 10 | IV | Single dose |
| 2 | 8 | Kadcyla | 2.5 | 10 | IV | Single dose |
| 3 | 8 | Kadcyla | 5 | 10 | IV | Single dose |
| 4 | 8 | α-ADC composition 1 | 1.25 | 10 | IV | Single dose |
| 5 | 8 | α-ADC composition 1 | 2.5 | 10 | IV | Single dose |
| 6 | 8 | α-ADC composition 1 | 5 | 10 | IV | Single dose |

TABLE 25

Study design in NCI-N87 model

| Group | N | Treatment | Dose (mg/kg) | Dosing Volume (µL/g)^{b} | Dosing Route | Schedule^{c} |
|---|---|---|---|---|---|---|
| 1 | 8 | Vehicle | -- | 10 | IV | Single dose |
| 2 | 8 | Kadcyla | 5 | 10 | IV | Single dose |
| 3 | 8 | Kadcyla | 15 | 10 | IV | Single dose |
| 4 | 8 | α-ADC composition 1 | 1.67 | 10 | IV | Single dose |
| 5 | 8 | α-ADC composition 1 | 5 | 10 | IV | Single dose |
| 6 | 8 | α-ADC composition 1 | 15 | 10 | IV | Single dose |

Treatment effects are calculated as follows: T/C(%) = (mean RTV of treated group)/ (mean RTV of control group) ×100%; TGI = [1-(mean tumor volume at the end of administration in a treated group - mean tumor volume at the beginning of administration in this treated group)/(mean tumor volume at the end of treatment in the control group - mean tumor volume at the beginning of treatment in the control group)] × 100%.

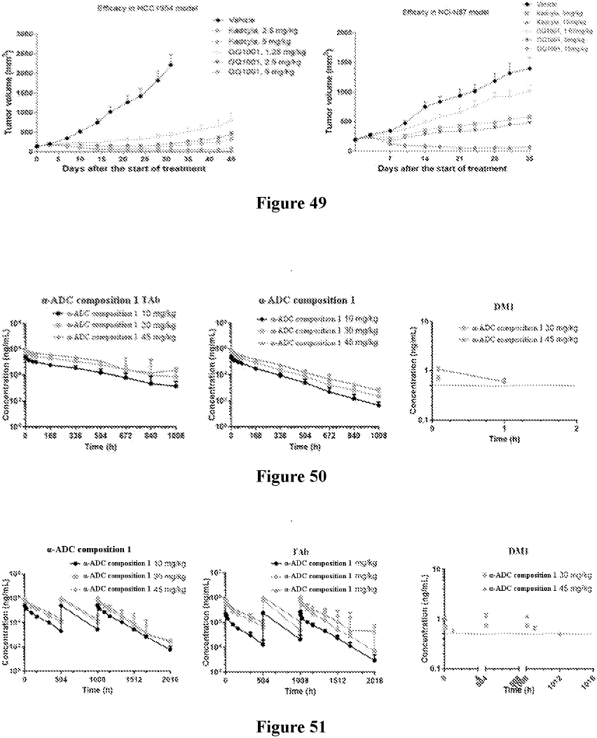

Result: In HCC1954 human breast cancer xenograft model, the therapeutic efficacy of α-ADC composition 1 (DAR 1.79) as a single agent was evaluated, and compared with Kadcyla (DAR~3.5) as the positive control. The results of tumor sizes in different groups at different time points after treatment started are shown in the FIG. 49. Compared with the vehicle group, treatment with the test article α-ADC composition 1 at 1.25, 2.5 mg/kg and 5 mg/kg (T/C = 18.83%, 9.12% and 2.12%, respectively; TGI = 86.61%, 96.97% and 104.43%, respectively; p = 0.002, 0.001 and 0.001, respectively) and Kadcyla at 2.5 mg/kg and 5 mg/kg (T/C = 6.47% and 1.57%, respectively; TGI = 99.79% and 105.02%, respectively; p = 0.001 and 0.001, respectively) produced a significant antitumor activity; their mean tumor sizes were 418, 202, 47, 144 and 35 mm³, respectively at the same time. α-ADC composition 1 and Kadcyla at the same dose level produced a similar anti-tumor activity in the HCC1954 xenograft model, p value was 0.791 (5 mg/kg) and 0.701 (2.5 mg/kg).

In NCI-N87 human gastric cancer xenograft model, the therapeutic efficacy of α-ADC composition 1 as a single agent was evaluated. The results of tumor sizes in different groups at different time points after tumor inoculation are shown in the FIG. 49. Compared with the vehicle group, treatment with the test article α-ADC composition 1 at 5 mg/kg, 15 mg/kg (T/C = 34.13 % and 5.07%, respectively; TGI = 76.62% and 110.38%, respectively; p = 0.010, 0.001, respectively) and Kadcyla at 5 mg/kg, 15 mg/kg (T/C = 41.61% and 4.66%, respectively; TGI = 67.93% and 110.87%, respectively; p = 0.022, 0.001, respectively) produced significant antitumor activity; their mean tumor sizes were 475, 71, 579 and 65 mm³, respectively at the same time. The tumor volume of α-ADC composition 1 at 1.67 mg/kg was 1,025 mm³ (T/C = 73.77%, TGI = 30.67%, p = 0.509) at day 35 post treatment. α-ADC composition 1 and Kadcyla at the same dose level produced a similar antitumor activity, p value was 0.791 (5 mg/kg) and 1.000 (15 mg/kg), respectively.

### Example 10: Pharmacokinetic Characteristics of α-ADCs

Objective: The objectives of this study is to evaluate the pharmacokinetic characteristics of α-ADCs after single administration in cynomolgus monkeys.

Study Design: A total of 24 Cynomolgus Monkeys (3 animals/gender/group, total 4 groups) were given single intravenous infusion (20 min/dose/monkey) of vehicle (α-ADC composition 1 preparation buffer) at 0 mg/kg, or α-ADC composition 1 at 10, 30 and 45 mg/kg. Animals were necropsied after 6-week observation period on Day 43. Details of number of animals and dose levels are given in Table 26.

TABLE 26

Groups and dose levels

| Groups | Number of animals | | Treatment | Dose levels |
|---|---|---|---|---|
| | ♂ | ♀ | | mg/kg |
| 1 | 3 | 3 | Vehicle | 0 |
| 2 | 3 | 3 | α-ADC composition 1 | 10 |
| 3 | 3 | 3 | α-ADC composition 1 | 30 |
| 4 | 3 | 3 | α-ADC composition 1 | 45 |

Blood collection time point include: 0 h, 5 min, 1 h, 4 h, 8 h, 24 h, 48 h, 72 h, 168 h, 336 h, 504 h, 672 h, 840 h and 1008 h.

Result: Summarized pharmacokinetic data were present in FIG. 50.

After IV infusion, serum concentration of α-ADC composition 1 and α-ADC composition 1 TAb generally peaked rapidly and declined in a roughly biphasic manner, and α-ADC composition 1 declined faster than TAb in the α-ADC composition 1 groups. The exposures of α-ADC composition 1 and TAb (mean Cₘₐₓ, AUC₀₋ₜ and AUC_{0-∞}) increased in an approximate dose-proportional manner. Little to no prolongation in the mean MRT was noted as the dose increased for α-ADC composition 1. No apparent gender difference was noted in Cₘₐₓ, AUC₀₋ₜ or AUC_{0-∞} for α-ADC composition 1 and α-ADC composition 1 TAb and α-ADC composition 1 mAb. α-ADC composition 1 T_{½} was 3.4 ~ 4.1 days, α-ADC composition 1 TAb T_{½} was 5.2 ~ 8.6 days.

DM1 was detected slightly higher than LLOQ at 5 min post dose in ≥30 mg/kg α-ADC composition 1 groups and at 1h post dose in 45 mg/kg α-ADC composition 1 group only. Mean Cₘₐₓ and AUC₀₋ₜ of DM1 increased with the increased dose from 30 to 45 mg/kg.

### Example 11: Toxicity and Potential Target Organ(s) of α-ADCs

Objective: The objectives of this study include: i) to assess the toxicity and potential target organ(s) after repeated intravenous infusion (once every 3-week for a total of 3 times) of α-ADCs in a 7-week main phase in cynomolgus monkeys, and to evaluate reversibility of observed toxicity or potential delayed toxicity after a 6-week recovery period in order to support the study design of subsequent toxicity studies and clinical trials. ii) to characterize the toxicokinetics of α-ADCs in cynomolgus monkeys.

Study Design: Forty cynomolgus monkeys (5/gender/group, total 4 groups) were randomly assigned into the study, and were administrated with α-ADC composition 1 (10, 30 and 45 mg/kg) or vehicle (0 mg/kg), once every 3-week for a total of 3 times in a 7-week dosing phase. At the end of the dosing phase, 3 animals/gender/group were necropsied, and the remaining 2 animals/gender/group were necropsied after a 6-week recovery phase. The results were shown in Table 27.

TABLE 27

Groups and dose levels

| Groups | Number of animals | | Treatment | Dose levels |
|---|---|---|---|---|
| | ♂ | ♀ | | mg/kg |
| Main Phase | | | | |
| 1 | 3 | 3 | Vehicle | 0 |
| 2 | 3 | 3 | α-ADC composition 1 | 10 |
| 3 | 3 | 3 | α-ADC composition 1 | 30 |
| 4 | 3 | 3 | α-ADC composition 1 | 45 |
| Recovery phase | | | | |
| 1 | 2 | 2 | Vehicle | 0 |
| 2 | 2 | 2 | α-ADC composition 1 | 10 |
| 3 | 2 | 2 | α-ADC composition 1 | 30 |
| 4 | 2 | 2 | α-ADC composition 1 | 45 |

All dose levels in the table are nominal.

Result: Summarized toxicokinetic data were present in FIG. 51.

After IV infusion of α-ADC composition 1 (10, 30, or 45 mg/kg), serum α-ADC composition 1 and TAb concentration generally peaked rapidly and declined in a roughly biphasic manner, respectively, then decreased with the time elapse, and α-ADC composition 1 declined faster than TAb both on Day 1 and Day 43. The average serum α-ADC composition 1 concentration was close to but slightly lower than that of TAb serum concentration. α-ADC composition 1 and TAb exposures (mean Cₘₐₓ and AUC₀₋ₜ) increased approximately in a dose-proportional manner. No gender difference was noted in terms of α-ADC composition 1 and TAb exposures. Based on exposure (AUC₀₋ₜ) on Day 1, the mean accumulation ratio on Day 43 was around 1.0 for both α-ADC composition 1 and TAb at all doses, demonstrating no apparent drug accumulation after repeated doses.

On Days 1 and 43, DM1 was detected slight higher than the LLOQ in three time points (5 min, 1 h and 4 h) post dose at the most in 30 and 45 mg/kg α-ADC composition 1-treated groups only. Mean Cₘₐₓ and AUC₀₋ₜ of DM1 increased with the increased dose from 30 to 45 mg/kg.

The highest non-severe toxicity dose (HNSTD) was determined as 45 mg/kg.

Noteworthy findings were shown as below.

45 mg/kg: decreases in PLT (males), prolongation in APTT (males), increases in AST and/or ALT was periodically noted at 7 days post dose in each dosing cycle, and recovered thereafter. Decreases in GLOB; decreases in A/G were observed in dosing phase. Increased spleen weights (absolute&relative) were present in both genders. Histopathology changes consisted of Kupffer cells hypertrophy in liver; hypercellularity and increased single cell necrosis in red pulp of spleen; epithelial cell necrosis in cornea (single male) and the Brunner’s gland in duodenum; increased mitotic figures of Kupffer cells in liver, spleen histiocytes, Brunner’s gland epithelium in duodenum (females), corneal epithelium (single male), esophagus squamous epithelium (male). Perivascular chronic inflammation and fibrosis were present in the administration site of one female. All above changes were not observed at the end of recovery phase, whereas bilateral increased mesangial matrix in glomeruli was noted in one male, meanwhile ADA was detected starting from Day 43, therefore the change was considered immunogenicity-related.

In conclusion, under the current study conditions, repeated intravenous infusion (once every 3 weeks, total 3 doses) with α-ADC composition 1 at 10, 30 and 45 mg/kg could be tolerated in cynomolgus monkeys at a dose up to 45 mg/kg. Potential target organs/tissues were liver, spleen, epithelium (duodenum, esophagus, and cornea), and administration site.

Compared with Kadcyla, α-ADC composition 1 release much less DM1 in circulation which may cause less side effects. Detailed comparison was shown in Table 28. α-ADC composition 1 DM1 Cₘₐₓ is ~1% that of T-DM1 at 30 mg/kg dose; α-ADC composition 1 DM1 AUC₀₋ₜ is ~0.002% that of T-DM1 at 30 mg/kg dose.

TABLE 28

DM1 TK parameters of α-ADC composition 1 and T-DM1 after repeated dose in Cynomolgus Monkeys (Mean)

| Parameters | | α-ADC composition 1 10 mg/kg | | Kadcyla^{∗} 10 mg/kg | | α-ADC composition 1 30 mg/kg | | Kadcyla^{∗} 30 mg/kg | |
|---|---|---|---|---|---|---|---|---|---|
| | | Male | Female | Male | Female | Male | Female | Male | Female |
| AUC₀₋ₜ (day•ng/ mL) | Cycle 1 | - | - | 19.8 | 19.8 | 0.00125 | 0.00105 | 53.7 | 65.1 |
| | Cycle ¾ | - | - | 23.7 | 22.5 | 0.00124 | 0.00124 | 69.4 | 92.8 |
| C ₘₐₓ (ng/mL) | Cycle 1 | - | - | 20.8 | 22.5 | 0.725 | 0.608 | 61.8 | 66.0 |
| | Cycle ¾ | - | - | 23.4 | 24.4 | 0.719 | 0.725 | 75.9 | 76.1 |

- : not detected; Cycle 3 is for α-ADC composition 1, Cycle 4 is for Kadcyla
^{∗}: Kadcyla BLA PHARMACOLOGY REVIEW(S), APPLICATION NUMBER:125427Orig1s000

Representative pathology assessment items of α-ADCs and Kadcyla were shown in Table 29.

Pathology assessments identified liver toxicity and peripheral neuropathy as major DM1-related toxicities. α-ADC composition 1 HNSTD dose (45 mg/kg) displays significantly milder severity in tissue pathology in comparison with Kadcyla at 10 mg/kg. Because α-ADC composition 1 and Kadcyla at the same dose level produced a similar antitumor activity, and the HNSTD of α-ADC composition 1 was 4.5 fold of Kadcyla, so the treatment window of α-ADC composition 1 should be much wider than Kadcyla.

TABLE 29

Representative pathology assessment items of α-ADCs and Kadcyla

| Representative pathology assessment items | | | α-ADC composition 1 45 mg/kg (HNSTD) | | Kadcyla^{∗} 10 mg/kg (HNSTD) | |
|---|---|---|---|---|---|---|
| | | | Male | Female | Male | Female |
| Main Phase (n=3 / gender) | Organ Weight | Body Weight | - | - | -7.1% | -12.5% |
| | | Thymus | - | - | -31.2% | -63.7% |
| | | Salivary Gland | - | - | -24.6% | -1.9% |
| | Spinal Cord | Axonal Degeneration, Dorsal Funiculus | - | - | 3/3 | - |
| | Sciatic Nerve | Axonal Degeneration | - | - | ⅔ | ⅓ |
| | | Hypertrophy/Hyperplasia, Schwan Cells | - | - | - | - |
| | Lung | Infiltrate, Mononuclear Cell, Interstitium | - | - | ⅓ | ⅓ |
| | Kidney | Infiltrate, Mononuclear Cell | - | - | ⅔ | ⅓ |
| | | Mitoses/Arrested Metaphase, Tubular Epithelial Cells | - | - | ⅓ | - |
| | Liver | Multinucleated Hepatocytes | - | - | | |
| | | Hypertrophy, Kupffer Cells | ⅔ | 3/3 | 3/3 | 3/3 |
| | | increased mitotic figure/ Mitoses/Arrested Metaphase | 3/3 | 3/3 | 3/3 | 3/3 |
| | | Increased Sinusoidal Leukocytes | - | - | 3/3 | ⅓ |
| | Spleen | Lymphoid Depletion, Follicular Center | - | - | - | - |
| | | Mitoses/Arrested Metaphase, Reticuloendothelial Cells | - | - | 3/3 | ⅓ |
| | Thymus | Lymphoid Depletion | - | - | ⅔ | 3/3 |
| Recovery phase (n=2 / gender) | Spinal Cord | Axonal Degeneration, Dorsal Funiculus | - | - | ½ | ½ |
| | Sciatic Nerve | Axonal Degeneration | - | - | ½ | ½ |
| | | Hypertrophy/Hyperplasia, Schwan Cells | - | - | - | - |
| | Liver | Multinucleated Hepatocytes | - | - | - | - |
| | | Microgranuloma(s) | - | - | - | 2/2 |
| | | Infiltrate, Neutrophilic | - | - | - | - |

^{∗}: Kadcyla BLA PHARMACOLOGY REVIEW(S), APPLICATION NUMBER:125427Orig1s000

It will be understood by a person skilled in the art that many amendments and modifications can be done to the present disclosure without departing its spirits and scope. The embodiments described herein are only provided as examples and should not be construed as limitation. The true scope and spirits are defined by the claims and the description and examples are illustrative only.

1-16. (canceled)

17. A process of making a compound of formula (XI), (XII), (XIII) or (XIV): 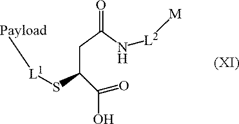 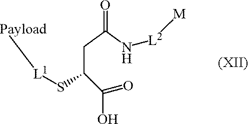 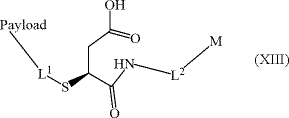 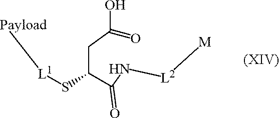
wherein
Payload is a cytotoxin moiety containing one or more chiral centers; L¹ is not present, or is a bivalent group being one or more selected from the group consisting of C₁₋₁₀ alkylene, C₃₋₁₀ cycloalkylene, C₆-₁₀ arylene, 4 to 10 membered heterocyclylene, 5 to 10 membered heteroarylene, —NH—, —(CO)—, —NH(CO)— and —(CO)NH—;
L² is a bond, or is a bivalent group being one or more selected from the group consisting of C₁₋₁₀ alkylene, C₃₋₁₀ cycloalkylene, C₆-₁₀ arylene, 4 to 10 membered heterocyclylene, 5 to 10 membered heteroarylene, —NH—, —(CO)—, —NH(CO)— and —(CO)NH—; and
M comprises a ligase recognition motif,
wherein the compound is in predominantly, e.g., in pure, or substantially pure isomeric form, e.g., substantially free of other isomeric forms, e.g., having a purity of greater than 90%, e.g., greater than 95%, e.g., greater than 98%, e.g., a least 99%, comprising
(i) synthesizing a mixture of compounds of formulae (XI), (XII), (XIII) and (XIV);
(ii) subjecting the mixture to chromatography so as to obtain the target compound(s),
wherein each of the target compound(s) are obtained in a separate product, or two target compounds are obtained in a second mixture; and
(iii) optionally recovering an eluate collected in step (ii) comprising a third mixture, wherein the third mixture comprises one or more additional target compounds which are different from those separated in step (2), and subjecting the eluate recovered to chromatography to separate the additional target compound(s).

18. The process of claim 17 wherein the chromatography is reverse-phase chromatography.

19-20. (canceled)

21. A process for separating one or more target compound(s) from Mixture 1 comprising four compounds, each of the four compounds comprising Moiety 1, Moiety 2 and Moiety 3;
wherein Moiety 1 has a ring-opened thiosuccinimide structure selected from formulae (I) to (IV): 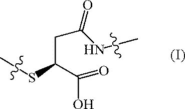 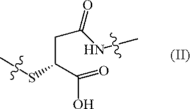 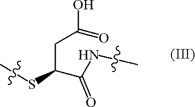 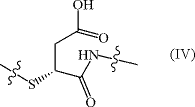
wherein
the thiol group and the amido group in Moiety 1 constitutes two linking sites, through one of which Moiety 2 is linked to Moiety 1, and through the other of which Moiety 3 is linked to Moiety 1;
Moiety 2 contains one or more chiral centers;
Moiety 1 in the four compounds is different;
Moiety 3 is the rest moiety of the molecule;
the weight of Moiety 3 is no more than 1900;
the one or more target compounds are selected from the said four compounds comprised by Mixture 1;
the process comprising the following steps (1) and (2)
(1) providing Mixture 1;
(2) subjecting Mixture 1 to chromatography so as to obtain the target compound(s), wherein a. each of the target compound(s) are obtained in a separate product, or b. two target compounds are obtained in a mixture, which is defined as Mixture 2.

22. The process of claim 21, further comprises the steps (3) and (4)
(3) recovering an eluate collected in step (2) comprising Mixture 3, wherein Mixture 3 comprises one or more additional target compounds which are different from those separated in step (2);
(4) subjecting the eluate recovered in step (3) to chromatography to separate the additional target compound(s).

23. The process of claim 21, wherein
in step (2) and step (4), four target compounds are obtained, wherein two target compounds are obtained in separate products in step (2) and the other two target compounds are obtained in separate products in step (4);
preferably, Moiety 1 in the two target compounds obtained in separate products in step (2) each have the structure of formula (I) or (II); and Moiety 1 in the two target compounds obtained in separate products in step (4) each have the structure of formula (III) or (IV).

24. The process of claim 21, wherein
in step (2), four target compounds are obtained, wherein two target compounds are obtained in separate products and the remaining two target compounds are obtained in Mixture 2; preferably, Moiety 1 in the two target compounds obtained in separate products in step (2) each have the structure of formula (I) or (II); and Moiety 1 in the two target compounds obtained in Mixture 2 each have the structure of formula (III) or (IV).

25. The process of claim 21, wherein
the chromatography in step (2) is reverse-phase chromatography; and/or
the stationary phases used in the reverse-phase chromatography in step (2) is selected from species of alkyl-bonded silica gel; and/or
the mobile phase in step (2) are as follows:
Eluent A: Water optionally containing Acidic agent 1;
Eluent B: Organic solvent 1 optionally containing Acidic agent 2;
provided that at least one of Acidic agent 1 and Acidic agent 2 is present;
separation range: B in gradient from about 0%-30% to about 30%-100%, with the remainder being A;
wherein the Acidic agents 1 and 2 are independently an inorganic acid or an organic acid.

26. The process of claim 21, wherein
Acidic agent 1 is selected from AcOH and L-tartaric acid (L-TA); preferably, Acidic agent 1 is AcOH; and/or
the amount of Acidic agent 1 is about 0.01% to about 1%, preferably about 0.05% to about 0.5%, more preferably about 0.1% to about 0.5%, most preferably about 0.1% to about 0.3%, especially 0.3%, based on the total volume of eluent A.

27. The process of claims 26, wherein
Acidic agent 2 is not present; or
the species of Acidic agent 2 is the same with Acidic agent 1; and/or
the amount of Acidic agent 2 is about 0.01% to about 1%, preferably 0.05% to about 0.5%, more preferably about 0.1% to about 0.5%, most preferably about 0.1% to about 0.3%, especially 0.3%, based on the total volume of eluent B; and/or
the total contents of Acidic agent 1 and Acidic agent 2 (if present) is about 0.005 to 0.06 M in the eluotropic composition.

28. The process of claim 26, wherein
when a target compound is being eluted, the total amount of Acidic agent 1 and Acidic agent 2 is no more than about 1%, preferably no more than about 0.8%, more preferably no more than about 0.6%, for example, no more than about 0.4%, especially no more than about 0.285%, based on the total volume of eluent A and eluent B.

29. The process of claim 21, wherein
the chromatography in step (4) is reverse-phase chromatography; and/or
the stationary phase of the reverse-phase chromatography is selected from species of alkyl-bonded silica gel; and/or
the mobile phase in step (4) are as follows:
Eluent C: Water optionally containing Acidic agent 3;
Eluent D: Organic solvent 2 optionally containing Acidic agent 4;
separation range: D in gradient from about 0%-30% to about 30%-100%, with the remainder being C;
wherein the Acidic agents 3 and 4 are independently an inorganic acid or an organic acid.

30. The process of claim 21, wherein
Acidic agent 3 is selected from TFA, phosphate buffer, ammonium acetate (AA), AcOH, H₃PO₄,
TEAP and L-tartaric acid (L-TA), preferably selected from TFA, phosphate buffer, AA and
TEAP, more preferably selected from TFA, phosphate buffer and TEAP, especially TFA; and/or the amount of Acidic agent 3 is about 0.01% to about 1%, preferably about 0.05% to about 0.5%, more preferably about 0.05% to about 0.3%, especially 0.1%, based on the total volume of eluent C.

31. The process of claim 30, wherein
Acidic agent 4 is not present; or
the species of Acidic agent 4 is the same with Acidic agent 3; and/or
the amount of Acidic agent 4 is about 0.01% to about 1%, preferably about 0.05% to about 0.5%, more preferably about 0.05% to about 0.3%, especially 0.1%, based on the total volume of eluent D; and/or
the total contents of Acidic agent 3 and Acidic agent 4 (if present) is about 0.01 to 0.25 M in the eluotropic composition.

32. The process of claim 30, wherein
at any time of the chromatographic process, the total amount of Acidic agent 3 and Acidic agent 4 is about 0.01% to about 1%, preferably about 0.05% to about 0.5%, more preferably about 0.05% to about 0.3%, especially 0.1%, based on the total volume of eluent C and eluent D.

33. The process of claim 21, wherein
each Moiety 2 in the four compounds comprised by Mixture 1 is identical; and
Moiety 2 has the structure of the following Formula (V): 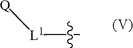
wherein Q is a group comprising at least one chiral centers; and L¹ is not present, or is a bivalent group being one or more selected from the group consisting of C₁₋₁₀ alkylene, C₃₋₁₀ cycloalkylene, C₆₋₁₀ arylene, 4 to 10 membered heterocyclylene, 5 to 10 membered heteroarylene, —NH—, —(CO)—, —NH(CO)— and —(CO)NH—;
preferably, Q is a C₅₋₅₀ hydrocarbyl, wherein
one or more “CH₂” structures in the hydrocarbyl are optionally replaced by —O—, —S—, —NH—, —C(═O)—, —S(═O)—, —S(═O)₂—, —NH(C═O)—, —C(═O)NH—, —NH—, S(═O)—,S(═O)NH—, —NHS(═O)₂— or —S(═O)₂NH—, given that a stable structure is formed;
one or more “CH” structures in the hydrocarbyl are optionally replaced by N or P, given that a stable structure is formed;
one or more carbon atoms, sulfur atoms, nitrogen atoms or phosphorus in Q are independently and optionally substituted by oxo (═O);
Q is optionally substituted by at least one substituents selected from R⁹, wherein each R⁹ is independently selected from R^{a1}, -OR^{a1}, -SRª¹, -NR^{a1}R^{b1}, -C(=O)OR^{a1}, -C(=O)NR^{a1}R^{b1}, -C(=O)R^{a1}, -S(=O)₂OR^{a1}, -S(=O)₂R^{a1}, -S(=O)₂NR^{a1}R^{b1}, -S(=O)R^{a1}, -C(=S)OR^{a1}, -C(=S)NR^{aq}R^{b1}, C(=S)R^{a1}, -P(=O)(OR^{a1})OR^{b1}, -C(=NR^{a1})NR^{b1}R^{c1}; and
R^{a1}, R^{b1} and R^{c1} are each independently selected from hydrogen and C₁₋₆ alkyl.

34. The process of claim 32, wherein
Moiety 2 has the structure of the following Formula (V-1): 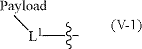
wherein Payload is selected from the group consisting of hydrogen and small molecule compounds comprising at least one chiral centers;
preferably, the small molecule is selected from enzyme inhibitors, enzyme activators, receptor modulators, toxins, glycans, PEG moieties, radionuclides, nucleic acids and analogues, tracer molecules, low molecular weight peptides, low molecular weight peptidomimetics, low molecular weight antibodies and antibody fragments.

35. The process of claim 21, wherein
each Moiety 3 in the four compounds comprised by Mixture 1 is identical; and/or
Moiety 3 has the structure of the following Formula (VI): 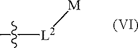
wherein L² is a bond, or is a bivalent group being one or more selected from the group consisting of C₁₋₁₀ alkylene, C₃₋₁₀ cycloalkylene, C₆-₁₀ arylene, 4 to 10 membered heterocyclylene, 5 to 10 membered heteroarylene, —NH—, —(CO)—, —NH(CO)— and —(CO)NH—;
M comprises: (1) Amino acid sequence 1 comprising 1-20 amino acids, (2) an optional polyethylene glycol (PEG) moiety, which comprises a “—(C₂H₄—O)ᵢ—” structure, i being an integer of 1 to 100, and (3) an optional group Y, which is bivalent and is selected from the group consisting of Cleavable sequence 1, spacer Sp1, and the combination thereof;
the cleavable sequence comprises Amino acid sequence 2 which can be cleaved by enzyme, and the cleavable sequence comprises 1-10 amino acids;
Sp1 is selected from the group consisting of a spacer sequence containing 1-20 amino acids, PAB, and the combination thereof.

36. The process of claim 34, wherein
L¹ is not present, or is selected from 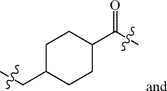 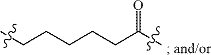
L² is selected from a bond, 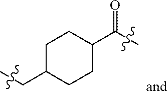 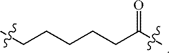
.

37. The process of claim 35, wherein
M is selected from the group consisting of a spacer sequence containing 1-20 amino acids, PAB, lysine, oligomeric glycine, oligomeric alanine, a mixture of oligomeric glycine/alanine having a degree of polymerization of 3-10, and the combination thereof; preferably,
M is L(G)ₙ, wherein L is Leucine (Leu); or
M is E(G)ₙ, wherein E is Glutamate (Glu); or
M is Gₙ;
G is glycine (Gly), and n is an integer of 3 to 10, especially 3.

38. The process of claim 37, wherein
M has an ionizable amino group; and Acidic agent 1 is AcOH; and
the amount of Acidic agent 1 is about 0.01% to about 1%, preferably about 0.05% to about 0.5%, more preferably about 0.1% to about 0.5%, more preferably about 0.1% to about 0.3%, especially 0.3%, based on the total volume of eluent A; and
Acidic agent 2 is not present; or Acidic agent 2 is present in an amount such that an aimed gradience in pH value of the mobile phase is obtained.

39. The process of claim 35, wherein
M has an ionizable amino group; and Acidic agent 3 is TFA; and
the amount of Acidic agent 3 is about 0.01% to about 1%, preferably about 0.05% to about 0.5%, more preferably about 0.05% to about 0.3%, especially 0.1%, based on the total volume of eluent C; and
Acidic agent 4 is not present; or Acidic agent 4 is present in an amount of about 0.01% to about 1%, preferably about 0.05% to about 0.5%, more preferably about 0.05% to about 0.3%, especially 0.1%, based on the total volume of eluent D; or Acidic agent 4 is present in an amount such that an aimed gradience in pH value of the mobile phase is obtained.

40. The process of claim 21,wherein
step (1) comprises a ring-opening reaction of a thiosuccinimide group, which affords the ring-opened thiosuccinimide structure of Moiety 1;
preferably, Mixture 1 is the reaction mixture resulted from the ring-opening reaction.

41. The process of claim 21, wherein
the four compounds comprised by Mixture 1 are isomeric compounds (i) to (iv), respectively 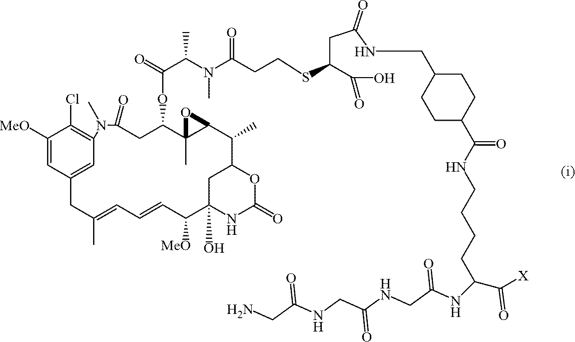 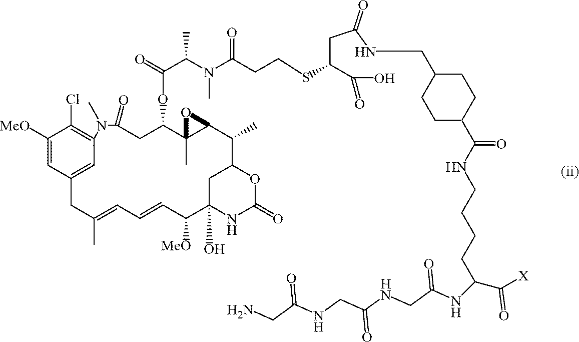 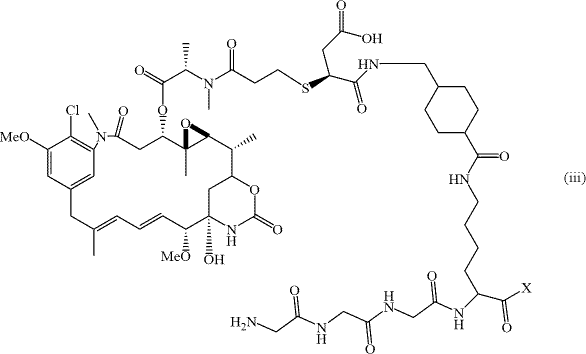 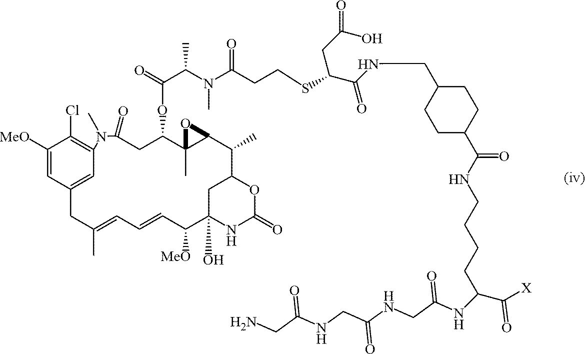
.

42. The process of claim 21, wherein
compounds (i) and (ii) are obtained in separate products in step (2); and compounds (iii) and (iv) are obtained in separate products in step (4).

43. The process of claim 21, wherein
in step (2), compounds (i) and (ii) are obtained in separate products, and compounds (iii) and (iv) are obtained in Mixture 2.

44. The process of claim 21, wherein
Mixture 1 is the reaction mixture resulted from the ring-opening reaction of the thiosuccinimide group in the following compound 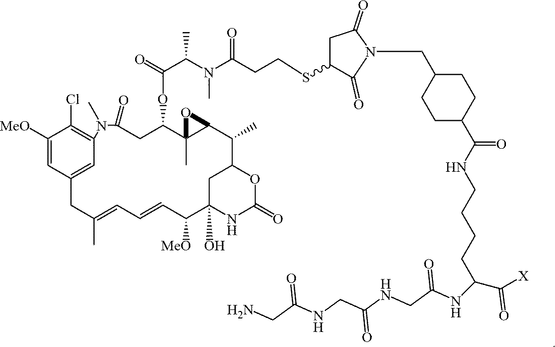
.

45-60. (canceled)

61. The process of claim 17, wherein the compound is selected from compounds of formula (i), (ii), (iii) or (iv) 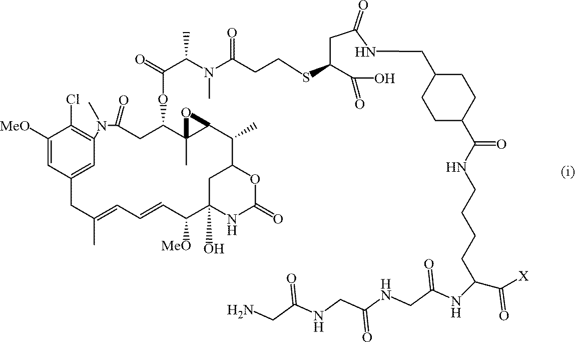 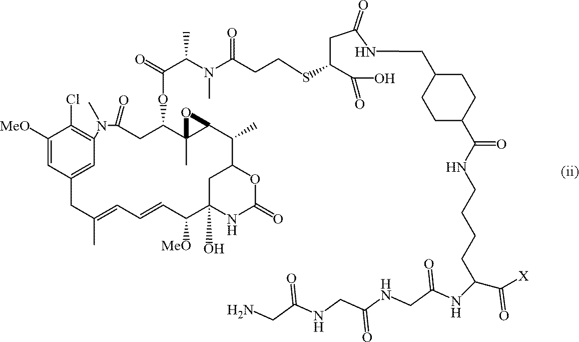 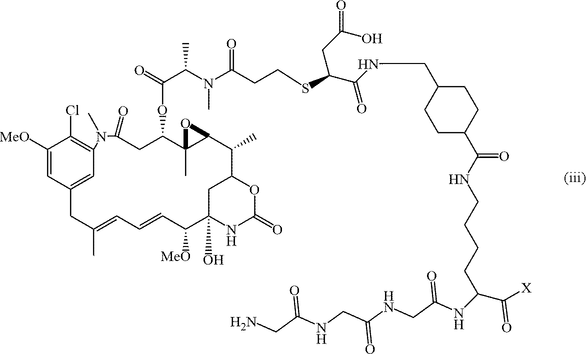 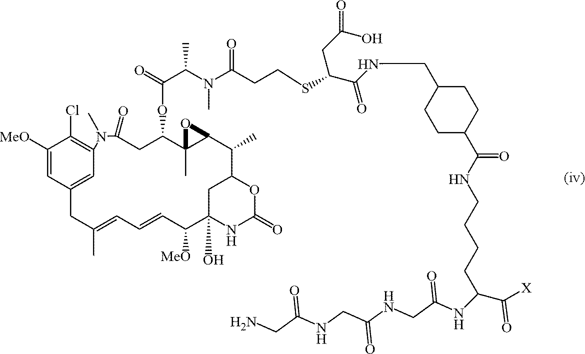
.
